SEQUENCE LISTING (207 sequences)

SEQ ID NO: 1 - protein, 105 aa, Homo sapiens
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 2 - protein, 27 aa, Homo sapiens
QDGNEEMGGITQTPYKVSISGTTVILT

SEQ ID NO: 3 - protein, 96 aa, Callithrix jacchus
QDGNEEMGDTTQNPYKVSISGTTVTLTCPRYDGHEIKWLVNSQNKEGHEDHLLLEDFSEMEQSGYYACLSKETPAEEASHYLYLKARVCENCVEVD

SEQ ID NO: 4 - protein, 27 aa, Callithrix jacchus
QDGNEEMGDTTQNPYKVSISGTTVTLT

SEQ ID NO: 5 - protein, 96 aa, Saguinus oedipus; identical to SEQ ID NO: 3

SEQ ID NO: 6 - protein, 27 aa, Saguinus oedipus
QDGNEEMGDTTQNPYKVSISGTTVTLT

SEQ ID NO: 7 - protein, 96 aa, Saimiri sciureus
QDGNEEIGDTTQNPYKVSISGTTVTLTCPRYDGQEIKWLVNDQNKEGHEDHLLLEDFSEMEQSGYYACLSKETPTEEASHYLYLKARVCENCVEVD

SEQ ID NO: 8 - protein, 27 aa, Saimiri sciureus
QDGNEEIGDTTQNPYKVSISGTTVTLT

SEQ ID NO: 9 - protein, 122 aa, Artificial
  Synthetic peptide
QVQLVQSGAEVKKPGESVKVSCKASGYTFTNYGMNWVRQAPGQGLEWMGWINTYTGEPTYADDFKGRVTMSSDTSTSTAYLEINSLRSDDTAIYYCARWSWSDGYYVYFDYWGQGTTVTVSS

SEQ ID NO: 10 - protein, 5 aa, Artificial
  Synthetic peptide
NYGMN

SEQ ID NO: 11 - protein, 17 aa, Artificial
  Synthetic peptide
WINTYTGEPTYADDFKG

SEQ ID NO: 12 - protein, 13 aa, Artificial
  Synthetic peptide
WSWSDGYYVYFDY

SEQ ID NO: 13 - protein, 113 aa, Artificial
  Synthetic peptide
DIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIK

SEQ ID NO: 14 - protein, 17 aa, Artificial
  Synthetic peptide
KSSQSVLDSSKNKNSLA

SEQ ID NO: 15 - protein, 7 aa, Artificial
  Synthetic peptide
WASTRES

SEQ ID NO: 16 - protein, 9 aa, Artificial
  Synthetic peptide
QQSAHFPIT

SEQ ID NO: 17 - protein, 250 aa, Artificial
  Synthetic peptide
QVQLVQSGAEVKKPGESVKVSCKASGYTFTNYGMNWVRQAPGQGLEWMGWINTYTGEPTYADDFKGRVTMSSDTSTSTAYLEINSLRSDDTAIYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIK

SEQ ID NO: 18 - protein, 505 aa, Artificial
  Synthetic peptide
QVQLVQSGAEVKKPGESVKVSCKASGYTFTNYGMNWVRQAPGQGLEWMGWINTYTGEPTYADDFKGRVTMSSDTSTSTAYLEINSLRSDDTAIYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 19 - protein, 505 aa, Artificial
  Synthetic peptide
QVQLVQSGAEVKKPGESVKVSCKASGYTFTNYGMNWVRQAPGQGLEWMGWINTYTGEPTYADDFKGRVTMSSDTSTSTAYLEINSLRSDDTAIYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 20 - protein, 505 aa, Artificial
  Synthetic peptide
QVQLVQSGAEVKKPGESVKVSCKASGYTFTNYGMNWVRQAPGQGLEWMGWINTYTGEPTYADDFKGRVTMSSDTSTSTAYLEINSLRSDDTAIYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 21 - protein, 122 aa, Artificial
  Synthetic peptide
QVQLVQSGAEVKKPGASVKVSCKASGYTFTNYGMNWVKQAPGQGLKWMGWINTYTGEPTYADDFKGRVTMTSDTSTSTAYLELHNLRSDDTAVYYCARWSWSDGYYVYFDYWGQGTTVTVSS

SEQ ID NO: 22 - protein, 5 aa, Artificial
  Synthetic peptide
NYGMN

SEQ ID NO: 23 - protein, 17 aa, Artificial
  Synthetic peptide
WINTYTGEPTYADDFKG

SEQ ID NO: 24 - protein, 13 aa, Artificial
  Synthetic peptide
WSWSDGYYVYFDY

SEQ ID NO: 25 - protein, 113 aa, Artificial; identical to SEQ ID NO: 13
  Synthetic peptide

SEQ ID NO: 26 - protein, 17 aa, Artificial
  Synthetic peptide
KSSQSVLDSSKNKNSLA

SEQ ID NO: 27 - protein, 7 aa, Artificial
  Synthetic peptide
WASTRES

SEQ ID NO: 28 - protein, 9 aa, Artificial
  Synthetic peptide
QQSAHFPIT

SEQ ID NO: 29 - protein, 250 aa, Artificial
  Synthetic peptide
QVQLVQSGAEVKKPGASVKVSCKASGYTFTNYGMNWVKQAPGQGLKWMGWINTYTGEPTYADDFKGRVTMTSDTSTSTAYLELHNLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIK

SEQ ID NO: 30 - protein, 505 aa, Artificial
  Synthetic peptide
QVQLVQSGAEVKKPGASVKVSCKASGYTFTNYGMNWVKQAPGQGLKWMGWINTYTGEPTYADDFKGRVTMTSDTSTSTAYLELHNLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 31 - protein, 505 aa, Artificial
  Synthetic peptide
QVQLVQSGAEVKKPGASVKVSCKASGYTFTNYGMNWVKQAPGQGLKWMGWINTYTGEPTYADDFKGRVTMTSDTSTSTAYLELHNLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 32 - protein, 505 aa, Artificial
  Synthetic peptide
QVQLVQSGAEVKKPGASVKVSCKASGYTFTNYGMNWVKQAPGQGLKWMGWINTYTGEPTYADDFKGRVTMTSDTSTSTAYLELHNLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 33 - protein, 122 aa, Artificial
  Synthetic peptide
QVQLVQSGAEVKKPGESVKVSCKASGYTFTNYGMNWVKQAPGQGLKWMGWINTYTGEPTYADDFKGRVTMTTDTSTSTAYMEIRNLRNDDTAVYYCARWSWSDGYYVYFDYWGQGTTVTVSS

SEQ ID NO: 34 - protein, 5 aa, Artificial
  Synthetic peptide
NYGMN

SEQ ID NO: 35 - protein, 17 aa, Artificial
  Synthetic peptide
WINTYTGEPTYADDFKG

SEQ ID NO: 36 - protein, 13 aa, Artificial
  Synthetic peptide
WSWSDGYYVYFDY

SEQ ID NO: 37 - protein, 113 aa, Artificial; identical to SEQ ID NO: 13
  Synthetic peptide

SEQ ID NO: 38 - protein, 17 aa, Artificial
  Synthetic peptide
KSSQSVLDSSKNKNSLA

SEQ ID NO: 39 - protein, 7 aa, Artificial
  Synthetic peptide
WASTRES

SEQ ID NO: 40 - protein, 9 aa, Artificial
  Synthetic peptide
QQSAHFPIT

SEQ ID NO: 41 - protein, 250 aa, Artificial
  Synthetic peptide
QVQLVQSGAEVKKPGESVKVSCKASGYTFTNYGMNWVKQAPGQGLKWMGWINTYTGEPTYADDFKGRVTMTTDTSTSTAYMEIRNLRNDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIK

SEQ ID NO: 42 - protein, 505 aa, Artificial
  Synthetic peptide
QVQLVQSGAEVKKPGESVKVSCKASGYTFTNYGMNWVKQAPGQGLKWMGWINTYTGEPTYADDFKGRVTMTTDTSTSTAYMEIRNLRNDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 43 - protein, 505 aa, Artificial
  Synthetic peptide
QVQLVQSGAEVKKPGESVKVSCKASGYTFTNYGMNWVKQAPGQGLKWMGWINTYTGEPTYADDFKGRVTMTTDTSTSTAYMEIRNLRNDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 44 - protein, 505 aa, Artificial
  Synthetic peptide
QVQLVQSGAEVKKPGESVKVSCKASGYTFTNYGMNWVKQAPGQGLKWMGWINTYTGEPTYADDFKGRVTMTTDTSTSTAYMEIRNLRNDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 45 - protein, 122 aa, Artificial
  Synthetic peptide
QVQLVQSGAEVKKPGESVKVSCKASGYTFTNYGMNWVKQAPGQGLKWMGWINTYTGEPTYADDFKGRVTMTSDTSTSTAYMEISSLRSDDTAVYYCARWSWSDGYYVYFDYWGQGTTVTVSS

SEQ ID NO: 46 - protein, 5 aa, Artificial
  Synthetic peptide
NYGMN

SEQ ID NO: 47 - protein, 17 aa, Artificial
  Synthetic peptide
WINTYTGEPTYADDFKG

SEQ ID NO: 48 - protein, 13 aa, Artificial
  Synthetic peptide
WSWSDGYYVYFDY

SEQ ID NO: 49 - protein, 113 aa, Artificial; identical to SEQ ID NO: 13
  Synthetic peptide

SEQ ID NO: 50 - protein, 17 aa, Artificial
  Synthetic peptide
KSSQSVLDSSKNKNSLA

SEQ ID NO: 51 - protein, 7 aa, Artificial
  Synthetic peptide
WASTRES

SEQ ID NO: 52 - protein, 9 aa, Artificial
  Synthetic peptide
QQSAHFPIT

SEQ ID NO: 53 - protein, 250 aa, Artificial
  Synthetic peptide
QVQLVQSGAEVKKPGESVKVSCKASGYTFTNYGMNWVKQAPGQGLKWMGWINTYTGEPTYADDFKGRVTMTSDTSTSTAYMEISSLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIK

SEQ ID NO: 54 - protein, 505 aa, Artificial
  Synthetic peptide
QVQLVQSGAEVKKPGESVKVSCKASGYTFTNYGMNWVKQAPGQGLKWMGWINTYTGEPTYADDFKGRVTMTSDTSTSTAYMEISSLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 55 - protein, 505 aa, Artificial
  Synthetic peptide
QVQLVQSGAEVKKPGESVKVSCKASGYTFTNYGMNWVKQAPGQGLKWMGWINTYTGEPTYADDFKGRVTMTSDTSTSTAYMEISSLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 56 - protein, 505 aa, Artificial
  Synthetic peptide
QVQLVQSGAEVKKPGESVKVSCKASGYTFTNYGMNWVKQAPGQGLKWMGWINTYTGEPTYADDFKGRVTMTSDTSTSTAYMEISSLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 57 - protein, 122 aa, Artificial
  Synthetic peptide
QVQLVQSGAEVKKPGESVKVSCKASGYTFTNYGMNWVKQAPGQGLEWMGWINTYTGETNYADKFQGRVTFTSDTSTSTAYMELRNLKSDDTAVYYCARWSWSDGYYVYFDYWGQGTTVTVSS

SEQ ID NO: 58 - protein, 5 aa, Artificial
  Synthetic peptide
NYGMN

SEQ ID NO: 59 - protein, 17 aa, Artificial
  Synthetic peptide
WINTYTGETNYADKFQG

SEQ ID NO: 60 - protein, 13 aa, Artificial
  Synthetic peptide
WSWSDGYYVYFDY

SEQ ID NO: 61 - protein, 113 aa, Artificial
  Synthetic peptide
DIVMTQSPDSMTVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLDIK

SEQ ID NO: 62 - protein, 17 aa, Artificial
  Synthetic peptide
KSSQSVLDSSTNKNSLA

SEQ ID NO: 63 - protein, 6 aa, Artificial
  Synthetic peptide
WASTRE

SEQ ID NO: 64 - protein, 9 aa, Artificial
  Synthetic peptide
QQSAHFPIT

SEQ ID NO: 65 - protein, 250 aa, Artificial
  Synthetic peptide
QVQLVQSGAEVKKPGESVKVSCKASGYTFTNYGMNWVKQAPGQGLEWMGWINTYTGETNYADKFQGRVTFTSDTSTSTAYMELRNLKSDDTAVYYCARWSWSDGYYVYFDYWGQGTTVTVSSGGGGSGGGGSGGGGSDIVMTQSPDSMTVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLDIK

SEQ ID NO: 66 - protein, 505 aa, Artificial
  Synthetic peptide
QVQLVQSGAEVKKPGESVKVSCKASGYTFTNYGMNWVKQAPGQGLEWMGWINTYTGETNYADKFQGRVTFTSDTSTSTAYMELRNLKSDDTAVYYCARWSWSDGYYVYFDYWGQGTTVTVSSGGGGSGGGGSGGGGSDIVMTQSPDSMTVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLDIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 67 - protein, 505 aa, Artificial
  Synthetic peptide
QVQLVQSGAEVKKPGESVKVSCKASGYTFTNYGMNWVKQAPGQGLEWMGWINTYTGETNYADKFQGRVTFTSDTSTSTAYMELRNLKSDDTAVYYCARWSWSDGYYVYFDYWGQGTTVTVSSGGGGSGGGGSGGGGSDIVMTQSPDSMTVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLDIKSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 68 - protein, 505 aa, Artificial
  Synthetic peptide
QVQLVQSGAEVKKPGESVKVSCKASGYTFTNYGMNWVKQAPGQGLEWMGWINTYTGETNYADKFQGRVTFTSDTSTSTAYMELRNLKSDDTAVYYCARWSWSDGYYVYFDYWGQGTTVTVSSGGGGSGGGGSGGGGSDIVMTQSPDSMTVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLDIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 69 - protein, 122 aa, Artificial; identical to SEQ ID NO: 57
  Synthetic peptide

SEQ ID NO: 70 - protein, 5 aa, Artificial
  Synthetic peptide
NYGMN

SEQ ID NO: 71 - protein, 17 aa, Artificial
  Synthetic peptide
WINTYTGETNYADKFQG

SEQ ID NO: 72 - protein, 13 aa, Artificial
  Synthetic peptide
WSWSDGYYVYFDY

SEQ ID NO: 73 - protein, 113 aa, Artificial
  Synthetic peptide
DIVMTQSPDSLSVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLEIK

SEQ ID NO: 74 - protein, 17 aa, Artificial
  Synthetic peptide
KSSQSVLDSSTNKNSLA

SEQ ID NO: 75 - protein, 7 aa, Artificial
  Synthetic peptide
WASTRES

SEQ ID NO: 76 - protein, 9 aa, Artificial
  Synthetic peptide
QQSAHFPIT

SEQ ID NO: 77 - protein, 250 aa, Artificial
  Synthetic peptide
QVQLVQSGAEVKKPGESVKVSCKASGYTFTNYGMNWVKQAPGQGLEWMGWINTYTGETNYADKFQGRVTFTSDTSTSTAYMELRNLKSDDTAVYYCARWSWSDGYYVYFDYWGQGTTVTVSSGGGGSGGGGSGGGGSDIVMTQSPDSLSVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLEIK

SEQ ID NO: 78 - protein, 505 aa, Artificial
  Synthetic peptide
QVQLVQSGAEVKKPGESVKVSCKASGYTFTNYGMNWVKQAPGQGLEWMGWINTYTGETNYADKFQGRVTFTSDTSTSTAYMELRNLKSDDTAVYYCARWSWSDGYYVYFDYWGQGTTVTVSSGGGGSGGGGSGGGGSDIVMTQSPDSLSVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 79 - protein, 505 aa, Artificial
  Synthetic peptide
QVQLVQSGAEVKKPGESVKVSCKASGYTFTNYGMNWVKQAPGQGLEWMGWINTYTGETNYADKFQGRVTFTSDTSTSTAYMELRNLKSDDTAVYYCARWSWSDGYYVYFDYWGQGTTVTVSSGGGGSGGGGSGGGGSDIVMTQSPDSLSVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 80 - protein, 505 aa, Artificial
  Synthetic peptide
QVQLVQSGAEVKKPGESVKVSCKASGYTFTNYGMNWVKQAPGQGLEWMGWINTYTGETNYADKFQGRVTFTSDTSTSTAYMELRNLKSDDTAVYYCARWSWSDGYYVYFDYWGQGTTVTVSSGGGGSGGGGSGGGGSDIVMTQSPDSLSVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 81 - protein, 122 aa, Artificial
  Synthetic peptide
QVQLVQSGAEVKKPGESVKVSCKASGYTFTNYGMNWVKQAPGQGLEWMGWINTYTGEPTYADKFQGRVTMTTDTSTSTAYMEIRNLRSDDTAVYYCARWSWSDGYYVYFDYWGQGTTVTVSS

SEQ ID NO: 82 - protein, 5 aa, Artificial
  Synthetic peptide
NYGMN

SEQ ID NO: 83 - protein, 17 aa, Artificial
  Synthetic peptide
WINTYTGEPTYADKFQG

SEQ ID NO: 84 - protein, 13 aa, Artificial
  Synthetic peptide
WSWSDGYYVYFDY

SEQ ID NO: 85 - protein, 113 aa, Artificial
  Synthetic peptide
DIVMTQSPDSLTVSLGERTTINCKSSQSVLDSSNNKNSLAWYQQKPGQPPKLLLSWASTRESGIPDRFSGSGSGTDFTLTIDGLQPEDSATYYCQQSAHFPITFGQGTRLEIK

SEQ ID NO: 86 - protein, 17 aa, Artificial
  Synthetic peptide
KSSQSVLDSSNNKNSLA

SEQ ID NO: 87 - protein, 7 aa, Artificial
  Synthetic peptide
WASTRES

SEQ ID NO: 88 - protein, 9 aa, Artificial
  Synthetic peptide
QQSAHFPIT

SEQ ID NO: 89 - protein, 250 aa, Artificial
  Synthetic peptide
QVQLVQSGAEVKKPGESVKVSCKASGYTFTNYGMNWVKQAPGQGLEWMGWINTYTGEPTYADKFQGRVTMTTDTSTSTAYMEIRNLRSDDTAVYYCARWSWSDGYYVYFDYWGQGTTVTVSSGGGGSGGGGSGGGGSDIVMTQSPDSLTVSLGERTTINCKSSQSVLDSSNNKNSLAWYQQKPGQPPKLLLSWASTRESGIPDRFSGSGSGTDFTLTIDGLQPEDSATYYCQQSAHFPITFGQGTRLEIK

SEQ ID NO: 90 - protein, 505 aa, Artificial
  Synthetic peptide
QVQLVQSGAEVKKPGESVKVSCKASGYTFTNYGMNWVKQAPGQGLEWMGWINTYTGEPTYADKFQGRVTMTTDTSTSTAYMEIRNLRSDDTAVYYCARWSWSDGYYVYFDYWGQGTTVTVSSGGGGSGGGGSGGGGSDIVMTQSPDSLTVSLGERTTINCKSSQSVLDSSNNKNSLAWYQQKPGQPPKLLLSWASTRESGIPDRFSGSGSGTDFTLTIDGLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 91 - protein, 505 aa, Artificial
  Synthetic peptide
QVQLVQSGAEVKKPGESVKVSCKASGYTFTNYGMNWVKQAPGQGLEWMGWINTYTGEPTYADKFQGRVTMTTDTSTSTAYMEIRNLRSDDTAVYYCARWSWSDGYYVYFDYWGQGTTVTVSSGGGGSGGGGSGGGGSDIVMTQSPDSLTVSLGERTTINCKSSQSVLDSSNNKNSLAWYQQKPGQPPKLLLSWASTRESGIPDRFSGSGSGTDFTLTIDGLQPEDSATYYCQQSAHFPITFGQGTRLEIKSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 92 - protein, 505 aa, Artificial
  Synthetic peptide
QVQLVQSGAEVKKPGESVKVSCKASGYTFTNYGMNWVKQAPGQGLEWMGWINTYTGEPTYADKFQGRVTMTTDTSTSTAYMEIRNLRSDDTAVYYCARWSWSDGYYVYFDYWGQGTTVTVSSGGGGSGGGGSGGGGSDIVMTQSPDSLTVSLGERTTINCKSSQSVLDSSNNKNSLAWYQQKPGQPPKLLLSWASTRESGIPDRFSGSGSGTDFTLTIDGLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 93 - protein, 122 aa, Artificial
  Synthetic peptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSS

SEQ ID NO: 94 - protein, 5 aa, Artificial
  Synthetic peptide
NYGMN

SEQ ID NO: 95 - protein, 17 aa, Artificial
  Synthetic peptide
WINTYTGEPTYADKFQG

SEQ ID NO: 96 - protein, 13 aa, Artificial
  Synthetic peptide
WSWSDGYYVYFDY

SEQ ID NO: 97 - protein, 113 aa, Artificial
  Synthetic peptide
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 98 - protein, 17 aa, Artificial
  Synthetic peptide
KSSQSVLDSSTNKNSLA

SEQ ID NO: 99 - protein, 7 aa, Artificial
  Synthetic peptide
WASTRES

SEQ ID NO: 100 - protein, 9 aa, Artificial
  Synthetic peptide
QQSAHFPIT

SEQ ID NO: 101 - protein, 250 aa, Artificial
  Synthetic peptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 102 - protein, 505 aa, Artificial
  Synthetic peptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 103 - protein, 505 aa, Artificial
  Synthetic peptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 104 - protein, 505 aa, Artificial
  Synthetic peptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 105 - protein, 505 aa, Artificial
  Synthetic peptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLSGGGGSQVQLVQSGAEVKKPGASVKVSCKASGYTFTNYGMNWVKQAPGQGLKWMGWINTYTGEPTYADDFKGRVTMTSDTSTSTAYLELHNLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIK

SEQ ID NO: 106 - protein, 505 aa, Artificial
  Synthetic peptide
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLSGGGGSQVQLVQSGAEVKKPGASVKVSCKASGYTFTNYGMNWVKQAPGQGLKWMGWINTYTGEPTYADDFKGRVTMTSDTSTSTAYLELHNLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIK

SEQ ID NO: 107 - protein, 505 aa, Artificial
  Synthetic peptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLSGGGGSQVQLVQSGAEVKKPGASVKVSCKASGYTFTNYGMNWVKQAPGQGLKWMGWINTYTGEPTYADDFKGRVTMTSDTSTSTAYLELHNLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIK

SEQ ID NO: 108 - protein, 511 aa, Artificial
  Synthetic peptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 109 - protein, 585 aa, Homo sapiens [sequence omitted]

SEQ ID NO: 110 - protein, 585 aa, Artificial
  Synthetic peptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAGTFTFHADICTLSEKERQIKKQTALVELVKHKPKATKEQLKAAMDDFAAFVEKCCKADDKETCFAEEGKKLVAASQAALGL

SEQ ID NO: 111 - protein, 585 aa, Artificial
  Synthetic peptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 112 - protein, 585 aa, Artificial
  Synthetic peptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 113 - protein, 585 aa, Artificial
  Synthetic peptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALGVDETYVPKEFNAETFTFHADICTLSEKERQIKKQTALVELVKHKPKATKEQLKAVMDKFAAFVEKCCKADDKETCFAEEGPKLVAASQAALGL

SEQ ID NO: 114 - protein, 585 aa, Artificial
  Synthetic peptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDKFAAFVEKCCKADDKETCFAEEGPKLVAASQAALGL

SEQ ID NO: 115 - protein, 585 aa, Artificial
  Synthetic peptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDKFAAFVEKCCKADDKETCFAEEGPHLVAASQAALGL

SEQ ID NO: 116 - protein, 585 aa, Artificial
  Synthetic peptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 117 - protein, 585 aa, Artificial
  Synthetic peptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDKFAAFVEKCCKADDKETCFAEEGPKLVAASKAALGL

SEQ ID NO: 118 - protein, 585 aa, Artificial
  Synthetic peptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDDFAAFVEKCCKADDKETCFAEEGPKLVAASKAALGL

SEQ ID NO: 119 - protein, 585 aa, Artificial
  Synthetic peptide
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 120 - protein, 585 aa, Artificial
  Synthetic peptide
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAGTFTFHADICTLSEKERQIKKQTALVELVKHKPKATKEQLKAAMDDFAAFVEKCCKADDKETCFAEEGKKLVAASQAALGL

SEQ ID NO: 121 - protein, 585 aa, Artificial
  Synthetic peptide
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 122 - protein, 585 aa, Artificial
  Synthetic peptide
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 123 - protein, 585 aa, Artificial
  Synthetic peptide
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALGVDETYVPKEFNAETFTFHADICTLSEKERQIKKQTALVELVKHKPKATKEQLKAVMDKFAAFVEKCCKADDKETCFAEEGPKLVAASQAALGL

SEQ ID NO: 124 - protein, 585 aa, Artificial
  Synthetic peptide
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDKFAAFVEKCCKADDKETCFAEEGPKLVAASQAALGL

SEQ ID NO: 125 - protein, 585 aa, Artificial
  Synthetic peptide
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDKFAAFVEKCCKADDKETCFAEEGPHLVAASQAALGL

SEQ ID NO: 126 - protein, 585 aa, Artificial
  Synthetic peptide
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 127 - protein, 585 aa, Artificial
  Synthetic peptide
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDKFAAFVEKCCKADDKETCFAEEGPKLVAASKAALGL

SEQ ID NO: 128 - protein, 585 aa, Artificial
  Synthetic peptide
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDDFAAFVEKCCKADDKETCFAEEGPKLVAASKAALGL

SEQ ID NO: 129 - protein, 585 aa, Artificial
  Synthetic peptide
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 130 - protein, 585 aa, Artificial
  Synthetic peptide
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAGTFTFHADICTLSEKERQIKKQTALVELVKHKPKATKEQLKAAMDDFAAFVEKCCKADDKETCFAEEGKKLVAASQAALGL

SEQ ID NO: 131 - protein, 585 aa, Artificial
  Synthetic peptide
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 132 - protein, 585 aa, Artificial
  Synthetic peptide
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 133 - protein, 585 aa, Artificial
  Synthetic peptide
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALGVDETYVPKEFNAETFTFHADICTLSEKERQIKKQTALVELVKHKPKATKEQLKAVMDKFAAFVEKCCKADDKETCFAEEGPKLVAASQAALGL

SEQ ID NO: 134 - protein, 585 aa, Artificial
  Synthetic peptide
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDKFAAFVEKCCKADDKETCFAEEGPKLVAASQAALGL

SEQ ID NO: 135 - protein, 585 aa, Artificial
  Synthetic peptide
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDKFAAFVEKCCKADDKETCFAEEGPHLVAASQAALGL

SEQ ID NO: 136 - protein, 585 aa, Artificial
  Synthetic peptide
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 137 - protein, 585 aa, Artificial
  Synthetic peptide
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDKFAAFVEKCCKADDKETCFAEEGPKLVAASKAALGL

SEQ ID NO: 138 - protein, 585 aa, Artificial
  Synthetic peptide
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDDFAAFVEKCCKADDKETCFAEEGPKLVAASKAALGL

SEQ ID NO: 139 - protein, 4 aa, Artificial
  Peptide linker
GGGG

SEQ ID NO: 140 - protein, 5 aa, Artificial
  Peptide linker
GGGGS

SEQ ID NO: 141 - protein, 5 aa, Artificial
  Peptide linker
GGGGQ

SEQ ID NO: 142 - protein, 6 aa, Artificial
  Peptide linker
PGGGGS

SEQ ID NO: 143 - protein, 6 aa, Artificial
  Peptide linker
PGGDGS

SEQ ID NO: 144 - protein, 6 aa, Artificial
  Peptide linker
SGGGGS

SEQ ID NO: 145 - protein, 9 aa, Artificial
  Peptide linker
GGGGSGGGS

SEQ ID NO: 146 - protein, 10 aa, Artificial
  Peptide linker
GGGGSGGGGS

SEQ ID NO: 147 - protein, 15 aa, Artificial
  Peptide linker
GGGGSGGGGSGGGGS

SEQ ID NO: 148 - protein, 14 aa, Artificial
  synthetic peptide
GSSTGAVTSGYYPN

SEQ ID NO: 149 - protein, 7 aa, Artificial
  synthetic peptide
GTKFLAP

SEQ ID NO: 150 - protein, 9 aa, Artificial
  synthetic peptide
ALWYSNRWV

SEQ ID NO: 151 - protein, 5 aa, Artificial
  synthetic peptide
IYAMN

SEQ ID NO: 152 - protein, 19 aa, Artificial
  synthetic peptide
RIRSKYNNYATYYADSVKS

SEQ ID NO: 153 - protein, 14 aa, Artificial
  synthetic peptide
HGNFGNSYVSFFAY

SEQ ID NO: 154 - protein, 14 aa, Artificial
  synthetic peptide
GSSTGAVTSGYYPN

SEQ ID NO: 155 - protein, 7 aa, Artificial
  synthetic peptide
GTKFLAP

SEQ ID NO: 156 - protein, 9 aa, Artificial
  synthetic peptide
ALWYSNRWV

SEQ ID NO: 157 - protein, 5 aa, Artificial
  synthetic peptide
KYAMN

SEQ ID NO: 158 - protein, 19 aa, Artificial
  synthetic peptide
RIRSKYNNYATYYADSVKD

SEQ ID NO: 159 - protein, 14 aa, Artificial
  synthetic peptide
HGNFGNSYISYWAY

SEQ ID NO: 160 - protein, 14 aa, Artificial
  synthetic peptide
GSSTGAVTSGYYPN

SEQ ID NO: 161 - protein, 7 aa, Artificial
  synthetic peptide
GTKFLAP

SEQ ID NO: 162 - protein, 9 aa, Artificial
  synthetic peptide
ALWYSNRWV

SEQ ID NO: 163 - protein, 5 aa, Artificial
  synthetic peptide
SYAMN

SEQ ID NO: 164 - protein, 19 aa, Artificial
  synthetic peptide
RIRSKYNNYATYYADSVKG

SEQ ID NO: 165 - protein, 14 aa, Artificial
  synthetic peptide
HGNFGNSYLSFWAY

SEQ ID NO: 166 - protein, 14 aa, Artificial
  synthetic peptide
GSSTGAVTSGYYPN

SEQ ID NO: 167 - protein, 7 aa, Artificial
  synthetic peptide
GTKFLAP

SEQ ID NO: 168 - protein, 9 aa, Artificial
  synthetic peptide
ALWYSNRWV

SEQ ID NO: 169 - protein, 5 aa, Artificial
  synthetic peptide
RYAMN

SEQ ID NO: 170 - protein, 19 aa, Artificial
  synthetic peptide
RIRSKYNNYATYYADSVKG

SEQ ID NO: 171 - protein, 14 aa, Artificial
  synthetic peptide
HGNFGNSYLSYFAY

SEQ ID NO: 172 - protein, 14 aa, Artificial
  synthetic peptide
RSSTGAVTSGYYPN

SEQ ID NO: 173 - protein, 7 aa, Artificial
  synthetic peptide
ATDMRPS

SEQ ID NO: 174 - protein, 9 aa, Artificial
  synthetic peptide
ALWYSNRWV

SEQ ID NO: 175 - protein, 5 aa, Artificial
  synthetic peptide
VYAMN

SEQ ID NO: 176 - protein, 19 aa, Artificial
  synthetic peptide
RIRSKYNNYATYYADSVKK

SEQ ID NO: 177 - protein, 14 aa, Artificial
  synthetic peptide
HGNFGNSYLSWWAY

SEQ ID NO: 178 - protein, 14 aa, Artificial
  synthetic peptide
GSSTGAVTSGYYPN

SEQ ID NO: 179 - protein, 7 aa, Artificial
  synthetic peptide
GTKFLAP

SEQ ID NO: 180 - protein, 9 aa, Artificial
  synthetic peptide
ALWYSNRWV

SEQ ID NO: 181 - protein, 5 aa, Artificial
  synthetic peptide
KYAMN

SEQ ID NO: 182 - protein, 19 aa, Artificial
  synthetic peptide
RIRSKYNNYATYYADSVKS

SEQ ID NO: 183 - protein, 14 aa, Artificial
  synthetic peptide
HGNFGNSYTSYYAY

SEQ ID NO: 184 - protein, 14 aa, Artificial
  synthetic peptide
RSSTGAVTSGYYPN

SEQ ID NO: 185 - protein, 7 aa, Artificial
  synthetic peptide
ATDMRPS

SEQ ID NO: 186 - protein, 9 aa, Artificial
  synthetic peptide
ALWYSNRWV

SEQ ID NO: 187 - protein, 5 aa, Artificial
  synthetic peptide
GYAMN

SEQ ID NO: 188 - protein, 19 aa, Artificial
  synthetic peptide
RIRSKYNNYATYYADSVKE

SEQ ID NO: 189 - protein, 14 aa, Artificial
  synthetic peptide
HRNFGNSYLSWFAY

SEQ ID NO: 190 - protein, 14 aa, Artificial
  synthetic peptide
GSSTGAVTSGYYPN

SEQ ID NO: 191 - protein, 7 aa, Artificial
  synthetic peptide
GTKFLAP

SEQ ID NO: 192 - protein, 9 aa, Artificial
  synthetic peptide
ALWYSNRWV

SEQ ID NO: 193 - protein, 5 aa, Artificial
  synthetic peptide
VYAMN

SEQ ID NO: 194 - protein, 19 aa, Artificial
  synthetic peptide
RIRSKYNNYATYYADSVKK

SEQ ID NO: 195 - protein, 14 aa, Artificial
  synthetic peptide
HGNFGNSYISWWAY

SEQ ID NO: 196 - protein, 14 aa, Artificial
  synthetic peptide
GSSTGAVTSGNYPN

SEQ ID NO: 197 - protein, 7 aa, Artificial
  synthetic peptide
GTKFLAP

SEQ ID NO: 198 - protein, 9 aa, Artificial
  synthetic peptide
VLWYSNRWV

SEQ ID NO: 199 - protein, 5 aa, Artificial
  synthetic peptide
SYAMN

SEQ ID NO: 200 - protein, 19 aa, Artificial
  synthetic peptide
RIRSKYNNYATYYADSVKG

SEQ ID NO: 201 - protein, 14 aa, Artificial
  synthetic peptide
HGNFGNSYVSWWAY

SEQ ID NO: 202 - protein, 14 aa, Artificial
  synthetic peptide
GSSTGAVTSGNYPN

SEQ ID NO: 203 - protein, 7 aa, Artificial
  synthetic peptide
GTKFLAP

SEQ ID NO: 204 - protein, 9 aa, Artificial
  synthetic peptide
VLWYSNRWV

SEQ ID NO: 205 - protein, 5 aa, Artificial
  synthetic peptide
KYAMN

SEQ ID NO: 206 - protein, 19 aa, Artificial
  synthetic peptide
RIRSKYNNYATYYADSVKD

SEQ ID NO: 207 - protein, 14 aa, Artificial
  synthetic peptide
HGNFGNSYISYWAY

(19) United States
(12) Patent Application Publication
(10) Pub. No.: US 2021/0301017 A1
(43) Pub. Date: Sep. 30, 2021

(54) PROLONGED ADMINISTRATION OF A BISPECIFIC ANTIBODY CONSTRUCT BINDING TO CD33 AND CD3

(71) Applicants: AMGEN RESEARCH (MUNICH) GMBH, Munich (DE); AMGEN INC., Thousand Oaks, CA (US)

(72) Inventors: Dirk Nagorsen, Thousand Oaks, CA (US); Bhakti Mehta, Thousand Oaks, CA (US); Peter Kufer, Munich (DE); Roman Kischel, Munich (DE); Sophia K. Khaldoyanidi, Thousand Oaks, CA (US)

(21) Appl. No.: 17/264,373

(22) PCT Filed: Jul. 29, 2019
(86) PCT No.: PCT/EP2019/070343

§ 371 Date: Jan. 29, 2021

Related U.S. Application Data
(60) Provisional application No. 62/712,147, filed on Jul. 30, 2018.

(51) Int. Cl. C07K 16/28; A61K 45/06; A61P 35/02

(52) U.S. Cl. CPC C07K 16/2809; A61K 2039/545; A61P 35/02; A61K 45/06

(57) ABSTRACT

The present invention provides a bispecific antibody construct comprising a first binding domain specifically binding to a target such as CD33 and a second binding domain specifically binding to an effector such as CD3 for use in a method for the treatment of myeloid leukemia, wherein the construct is administered in one or more treatment cycles of more than 14 days applying a step dosing comprising at least two steps, a treatment cycle optionally followed by a period without administration of the construct. Moreover, the invention provides a method for the treatment of myeloid leukemia comprising the administration of a therapeutically efficient amount of such bispecific antibody construct and the use of such bispecific antibody construct for the preparation of a pharmaceutical composition for the treatment of myeloid leukemia.

### FIELD OF THE INVENTION

The present invention relates to a bispecific antibody construct comprising a first binding domain specifically binding to a target such as CD33 and a second binding domain specifically binding to an effector such as CD3, preferably for use in a method for the treatment of myeloid leukemia, wherein the construct is administered for a period of more than 14 days optionally followed by a period of at least 7 days without administration of the construct. Moreover, the invention relates to a method for the treatment of myeloid leukemia comprising the administration of a therapeutically efficient amount of such bispecific antibody construct and the use of such bispecific antibody construct for the preparation of a pharmaceutical composition for the treatment of myeloid leukemia.

### BACKGROUND OF THE INVENTION

Bispecific antibody constructs such as BiTE® (bispecific T cell engager) antibody constructs are recombinant protein constructs made from two flexibly linked antibody derived binding domains. One binding domain of bispecific antibody constructs is specific for a selected tumor-associated surface antigen on target cells; the second binding domain is specific for CD3, a subunit of the T cell receptor complex on T cells. By their particular design BiTE® antibody constructs are uniquely suited to transiently connect T cells with target cells and, at the same time, potently activate the inherent cytolytic potential of T cells against target cells. The first generation of bispecific antibody constructs (see WO 99/54440 and WO 2005/040220) developed into the clinic as blinatumomab and solitomab. These bispecific antibody constructs are administered via continuous intravenous infusion. For example, blinatumomab is administered in B acute lymphoblastic leukemia as 4-week infusing with a lower initial dose in the 1st week and a higher dose in the remaining treatment for the 1st cycle and in all other cycles from start. Before starting a second cycle, there is a treatment-free period of two weeks. A similar administration schema has been used for solitomab which was administered as continuous intravenous infusion over at least 28 days with increasing doses and also a treatment-free period of two weeks between two cycles.

An important further development of the first generation of bispecific antibody constructs was the provision of bispecific antibody constructs binding to a context independent epitope at the N-terminus of the CD3ε chain of human and Callithrix jacchus, Saguinus oedipus or Saimiri sciureus (WO 2008/119567). Hence, such bispecific antibody constructs have become versatile means to address so-far unmet therapeutic needs.

One such need is an efficient and safe therapy of Acute Myeloid Leukemia (AML), in particular relapsed or refractory AML (r/r AML). Prognosis of patients with relapsed or refractory AML is poor as no standard salvage therapy exists except in the case of AML with specific mutations such IDH1/2 mutations. Most trials of investigational agents begin in r/r AML and have accrued a wide range of patients with different characteristics. Historical context for outcomes can be used as a reference for the development of future protocols and novel agents. Analysis of such historical context reveals that overall survival and event free survival were modest and decreased with subsequent salvage. Age, cytogenetics, antecedent disease, De novo/therapy-induced AML, duration of first remission, and platelet count were associated with survival. Importantly, in the vast majority of cases, patients, in particular a majority of r/r AML patients, are unable to achieve a sustained second or subsequent remission, i.e. a long-term amelioration or even cure of the disease. Hence, there is a need for further therapeutic means and an optimized use thereof.

CD33 is a sialic-acid-dependent cytoadhesion molecule known as a myeloid differentiation antigen found inter alia on AML blasts in most patients and leukemic stem cells Therefore, CD33 has been identified as a promising marker for myeloid leukemia and a target molecule in the treatment of such diseases. To this end, Mylotarg® (gemtuzumab ozogamcin), a cytotoxic antibiotic linked to a recombinant monoclonal antibody directed against the CD33 antigen present on leukemic myeloblasts, had been approved in the United States for patients with AML through accelerated approval. However, following the drug's failure to demonstrate clinical benefit in the confirmatory trial, and an increased risk of venoocclusive disease observed in the postmarketing setting, the drug was temporarily withdrawn voluntarily by the manufacturer from the United States market. Frequently reported toxicities observed with gemtuzumab ozogamcin included neutropenia and thrombocytopenia, and less frequently reported toxicities included events related to acute infusion-related reactions (anaphylaxis), hepatotoxicity, and veno-occlusive disease. In view of said side effects a CD33-based agent may have, a promising CD33×CD3 bispecific antibody construct has been proposed for use in the treatment of AML, wherein the administration duration within one treatment cycle was previously limited to a maximum of 14 days. Thereby, severe side effects such as neutropenia, especially agranulocytosis should be avoided. While avoiding severe side effects is already a major and important achievement in introducing a potent agent such as the CD33×CD3 bispecific antibody construct, it is highly desirable to best exploit its therapeutic potential at the same time to effectively and sustainably treat the disease. Hence, it is the objective of the present invention to provide an improved administration of the CD33×CD3 bispecific antibody construct which avoids side effects and best exploits the therapeutic potential of said construct at the same time.

### SUMMARY OF THE INVENTION

In a first aspect, the present invention refers to a bispecific antibody construct comprising a first binding domain specifically binding to CD33 and a second binding domain specifically binding to CD3 preferably for use in a method for the treatment of myeloid leukemia, wherein the bispecific antibody construct is administered in one or more treatment cycles, wherein at least one treatment cycle comprises more than 14 days of administration of the bispecific antibody construct in at least three different dosages applying at least two dosage steps, optionally followed by a period without administration of the bispecific antibody construct,
wherein the bispecific antibody construct is administered in at least one of the one or more treatment cycles according to a schedule comprising the following steps:
   (a) administration of a first dosage of the bispecific antibody construct, followed by
   (b) administration of a second dosage of the bispecific antibody construct, wherein said second dosage exceeds said first dose, followed by
   (c) administration of a third dosage of the bispecific antibody construct, wherein said third dosage exceeds said second dosage, optionally followed by
   (d) administration of a forth dosage of the bispecific antibody construct, wherein said optional forth dose exceeds said third dosage.
It is envisaged in one aspect of the present invention that the time of administering the bispecific antibody construct in one treatment cycle including all steps (a) to (c) or (d) is at least 15 days, preferably 15 to 60 days, more preferably 28 to 56 days, preferred 28 days.
It is envisaged in one aspect of the present invention that the first dosage in step (a) is at least 5 μg per day, preferably in the range of 5 to 20 μg per day, preferably 10 μg per day, the second dosage in step (b) is at least 30 μg per day, preferably in the range of 30 to 240 μg per day, preferably 60 or 240 μg per day and the third dosage in step (c) and optional forth dosage in step (d) of at least 240 μg per day, preferably 240 to 1500 μg per day, more preferably in the range of 480 to 960 μg per day.
It is envisaged in one aspect of the present invention that the period of administration of the first dosage in step (a) is 1 to 4 days, preferably 2 or 3 days, the period of administration of the second dosage in step (b) is 2 to 5 days, preferably 2 or 3 days, and the period of administration of the third dosage in step (c) and the optional forth dosage in step (d) together is 7 to 52 days, preferably 14 to 23 or 52 days, more preferably 22, 23 or 52 days.
It is envisaged in one aspect of the present invention that the treatment of the myeloid leukemia comprises two or more treatment cycles, preferably two, three, four, five, six or seven treatment cycles, whereof at least one, two, three, four five, six or seven treatment cycles comprise more than 14 days of bispecific antibody construct administration.
It is envisaged in one aspect of the present invention that the at least one treatment cycle is followed by a period without administration of the bispecific antibody construct, preferably at least 1, 2, 3, 4, 5, 6, 7, 8, 9, 10, 11, 12, 13 or 14 days without treatment.
It is envisaged in one aspect of the present invention that at least one treatment cycle is not followed by the period without administration of the construct.
It is envisaged in one aspect of the present invention that only the first cycle of the treatment comprises the administration according to step (a), whereas the following cycles start with the dosage according to step (b).
It is envisaged in one aspect of the present invention that the first binding domain of the bispecific antibody construct is a single chain bispecific antibody construct.
It is envisaged in one aspect of the present invention that the first binding domain of the bispecific antibody construct comprises groups of six CDRs selected from the group consisting of SEQ ID NOs: 10 to 12 and 14 to 16, 22 to 24 and 26 to 28, 34 to 36 and 38 to 40, 46 to 48 and 50 to 52, 58 to 60 and 62 to 64, 70 to 72 and 74 to 76, 82 to 84 and 86 to 88, 94 to 96 an 98 to 100, preferably 94 to 96 an 98 to 100.
It is envisaged in one aspect of the present invention that the second binding domain of the bispecific antibody construct comprises groups of six CDRs selected from the group consisting of SEQ ID NOs: 148-153, 154-159, 160-165, 166-171, 172-177, 178-183, 184-189, 190-195, 196-201 and 202-207, preferably 202-207.
It is envisaged in one aspect of the present invention that the bispecific antibody construct is a single chain construct comprising an amino acid sequence selected from the group consisting of SEQ ID NOs: 18, 19, 20, 30, 31, 32, 42, 43, 44, 54, 55, 56, 66, 67, 68, 78, 79, 80, 90, 91, 92, 102, 103, 104, 105, 106, 107 and 108, preferably selected from the group consisting of SEQ ID NOs: 104, 105, 106, 107 and 108, more preferably SEQ ID NO 104.
It is envisaged in one aspect of the present invention that the bispecific antibody construct is administered in combination with a PD-1 inhibitor, a PDL-1 inhibitor and/or one or more epigenetic factors selected from the group consisting of histone deacetylase (HDAC) inhibitors, DNA methyltransferase (DNMT) I inhibitors, hydroxyurea, Granulocyte-Colony Stimulating Factor (G-CSF), histone demethylase inhibitors and ATRA (All Trans-retinoic acid) and wherein:
   (a) the PD-1 inhibitor, a PDL-1 inhibitor and/or one or more epigenetic factors are administered prior to the administration of the bispecific antibody construct;
   (b) the PD-1 inhibitor, a PDL-1 inhibitor and/or one or more epigenetic factors are administered subsequent to the administration of the bispecific antibody construct; or
   (c) the PD-1 inhibitor, a PDL-1 inhibitor and/or one or more epigenetic factors and the bispecific antibody construct are administered simultaneously.
It is envisaged in one aspect of the present invention that the PD-1 inhibitor, a PDL-1 inhibitor and/or one or more epigenetic factors are administered prior to the administration of the bispecific antibody construct, preferably 1, 2, 3, 4, 5, 6, or 7 days prior to the administration of the bispecific antibody construct.
It is envisaged in one aspect of the present invention that the epigenetic factor is hydroxyurea.
It is envisaged in one aspect of the present invention that the myeloid leukemia is selected from the group consisting of acute myeloblastic leukemia, preferably relapsed or refractory acute myeloid leukemia, chronic neutrophilic leukemia, myeloid dendritic cell leukemia, accelerated phase chronic myelogenous leukemia, acute myelomonocytic leukemia, juvenile myelomonocytic leukemia, chronic myelomonocytic leukemia, acute basophilic leukemia, acute eosinophilic leukemia, chronic eosinophilic leukemia, acute megakaryoblastic leukemia, essential thrombocytosis, acute erythroid leukemia, polycythemia vera, myelodysplastic syndrome, acute panmyeloic leukemia, myeloid sarcoma, and mixed phenotypic acute leukemia.
It is envisaged in another aspect of the present invention that a method for the treatment of myeloid leukemia in a patient in need thereof is provided, the method comprising administering a therapeutically efficient amount of a bispecific antibody construct comprising a first binding domain specifically binding to CD33 and a second binding domain specifically binding to CD3 in one or more treatment cycles, wherein the at least one treatment cycle comprises more than 14 days of administration of the bispecific antibody construct in at least three different dosages applying at least two dosage steps,
wherein the bispecific antibody construct is administered in one treatment cycle according to a schedule comprising the following steps:
(a) administration of a first dosage of the bispecific antibody construct, followed by
(b) administration of a second dosage of the bispecific antibody construct, wherein said second dosage exceeds said first dosage, followed by
(c) administration of a third dosage of the bispecific antibody construct, wherein said third dosage exceeds said second dosage, optionally followed by
(d) administration of a forth dosage of the bispecific antibody construct, wherein said optional forth dosage exceeds said third dosage, optionally followed by a period of at without administration of the construct.
It is envisaged in another aspect of the present invention that the time of administering the bispecific antibody construct in one treatment cycle is at least 15 days, preferably 15 to 60 days, more preferably 28 to 56 days, more preferred 28 days.
It is envisaged in another aspect of the present invention that the first dosage in step (a) is at least 5 μg per day, preferably in the range of 5 to 20 μg per day, more preferably 10 μg per day, the second dosage in step (b) is at least 30 μg per day, preferably in the range of 30 to 240 μg per day, preferably 60 or 240 μg per day and the third dosage in step (c) and the optional forth dosage in optional step (d) is at least 240 μg per day, preferably in the range of 120 to 1500 μg per day, preferably 240 to 960 μg per day, more preferably 480 to 960 μg per day.
It is envisaged in another aspect of the present invention that the period of administration of the first dosage in step (a) is 1 to 4 days, preferably 2 or 3 days, the period of administration of the second dosage in step (b) is 2 to 5 days, preferably 2 or 3 days, and the period of administration of the third and the optional forth dose in step (c) and optional step (d), respectively, is 7 to 52 days, preferably 14 to 23 days, more preferably 22, 23, 50 or 52 days.
It is envisaged in another aspect of the present invention that the treatment of the myeloid leukemia comprises two or more treatment cycles, preferably 2, 3, 4, 5, 6 or 7 treatment cycles, whereof at least 1, 2, 3, 4, 5, 6 or 7 treatment cycles which each comprises more than 14 days of bispecific antibody construct administration.
It is envisaged in another aspect of the present invention that the treatment is followed by the period without administration of the bispecific antibody construct, preferably at least 1, 2, 3, 4, 5, 6, 7, 8, 9, 10, 11, 12, 13, or 14 days without treatment.
It is envisaged in another aspect of the present invention that the treatment is followed by the period of at least 14 days without administration of the bispecific antibody construct.
It is envisaged in another aspect of the present invention that only the first cycle of the treatment comprises the administration according to step (a), whereas the following cycles start with the dose according to step (b).
It is envisaged in another aspect of the present invention that the construct is a single chain bispecific antibody construct.

### BRIEF DESCRIPTION OF THE DRAWINGS

FIG. 1: O of a Phase I clinical study on CD33×CD3 bispecific antibody construct comprising the first 12 patient cohorts.

FIG. 2: Overview of anti-tumor activity with respect to the first 12 patient cohorts in a Phase I clinical study. Anti-tumor efficacy with respect to study cohort: 2 complete remissions (CRs) at 240 μg/d target dosage (third dosage within treatment cycle comprising three dosages), 1 CRi at 120 μg/d and 1 at 240 μg/d, 1 patient at 1.5 μg/d had a MLFS (<5% blasts, no hematologic recovery). The patient with a CR had bm blasts ˜5%-10% at baseline (estimated due to patchy disease) and down to 2.5% by day 29 by flow cytometry, with no morphologic evidence of residual AML and normo- to hypercellular marrow and, most importantly, recovery of peripheral blood counts. (Legend: CR: Complete Remission, CRi: Complete Remission with Incomplete Count Recovery, MLFS: Morphologic Leukemia-Free State).

FIG. 3: Overview on tumor response under CD33×CD3 bispecific antibody construct treatment: Change in bone marrow (BM) aspirate blast count [%] in dependence of CD33×CD3 dosage as indicator for tumor response.

FIG. 4: Overview of patient response in respective treatment cycles

FIG. 5: Phase 1 dose escalation study evaluating AMG 330 as a continuous IV infusion in patients with R/R AML showing step dosing for prolonged target dose exposure and mitigation of CRS side effects.

FIG. 6: Absolute neutrophil counts in peripheral blood of patients treated in cohorts 11 (240 μg), 12 (240 μg) and 13 (30 μg) during the DLT window are shown. Mean±SE is shown. G4 line (lower base line) and G3 line (upper base line) show grade 4 and 3 neutropenia by CTCAE.

FIG. 7: Platelet counts in peripheral blood of patients treated in cohorts 11 (10, 30 and 240 μg), 12 (240 μg) and 13 (30 μg) during the DLT window are shown. Mean±SE is shown. G4 line (lower base line) and G3 line (upper base line) show grade 4 and 3 neutropenia by CTCAE.

### DETAILED DESCRIPTION OF THE INVENTION

A bispecific antibody construct having market authorization, Blinatumomab, is given in the treatment of acute lymphoblastic leukemia (ALL) by continuous intravenous infusion for 4 weeks (5 μg/m²/day for the first week and 15 μg/m²/day thereafter), followed by two treatment-free weeks (i.e. one cycle) for up to five cycles. Doing so, the treatment eliminates the compartment of CD19⁺ cells, which is a compartment limited to the B cell linage.

In animal studies testing a CD33 specific BiTE®, consistent with the proposed mode of action for such molecule, transient myelosuppression was observed, including reductions in circulating neutrophils, platelets, and red cell mass. The decrease in leukocytes, along with the expected increase in activated animal studies, consistent with the proposed mode of action, resulted in transient myelosuppression including reductions in circulating neutrophils, platelets, and red cell mass. The decrease in leukocytes, along with the expected increase in activated T-lymphocytes and increase in cytokine levels were observed in all dose groups. Febrile neutropenia and neutropenia are common events observed in patients with hematological malignancies and prior combination chemotherapies.

Bleeding is a common and potentially serious complication of the treatment of AML, most often secondary to thrombocytopenia. Among bleeding complications, of particular importance is the disseminated intravascular coagulation (DIC) syndrome, due to the massive intravascular activation of blood coagulation with consumption of clotting factors and platelets, leading to severe hemorrhages. In adult patients with AML, 1% of lethal bleedings on day of admission have been observed, all in the presence of hyperleukocytosis or acute promyelocytic leukemia (APL). Recent data in patients with AML show a rate of hemorrhagic death of 9.9%. In addition, in this AML patient population, there may be a strong correlation between unresolved infection in the pancytopenic patient and terminal hemorrhage.

It is also well accepted that immunocompromised patients are susceptible to both common community-acquired and opportunistic infections. Infections are a major cause of morbidity and mortality in cancer patients and although certain cancers are intrinsically associated with immune compromise, the risk of infection is principally related to the intensity and duration of cytotoxic and immunosuppressive therapy.

However, in acute myeloid leukemia the situation is different compared to acute lymphoblastic leukemia. The myeloid compartment includes a broader spectrum of cell lineages that are necessary for survival. Therefore, it is not possible to simply transfer the administration scheme of blinatumomab in ALL to a treatment of AML using an AML specific bispecific antibody construct. For an efficient treatment of AML using a CD33⁺ cell eliminating therapy approach, the treatment needs to be long enough to be efficacious and short enough to be minimize toxicity on those cell types in the myeloid compartment that are essential for survival, e.g. in view of maintaining immunocompetence to rebut opportunistic infections. In addition, the dosage needs to be sufficient for efficacy as well.

This problem was solved, e.g., by providing a bispecific antibody construct comprising a first binding domain specifically binding to CD33 and a second binding domain specifically binding to CD3 (CD33/CD3) preferably for use in a method for the treatment of myeloid leukemia, wherein the bispecific antibody construct is administered in one or more treatment cycles, wherein one treatment cycle comprises more than 14 days of administration of the bispecific antibody construct in at least three different dosages applying at least two dosage steps, optionally followed by a period without administration of the construct.

Using an administration schedule in line with the present invention, applying at least a twofold step dosing with at least three increasing dosage levels, it is possible to efficiently eliminate myeloid leukemic cells during the more than 14 days of CD33/CD3 bispecific antibody construct (e.g. SEQ ID NO: 104) administration period, while still allowing the patient to recover the myeloid compartment in a period without administration of the construct between treatment cycles. Employing a target dosage, i.e. the maximum dosage of the last step within a treatment cycle, of at least 240 μg preferably enables a complete remission of the disease as demonstrated herein. At the same time, the step dosing according to the present invention preferably significantly reduces the risk of severe immunologic side effects such as a cytokine release syndrome or symptoms thereof despite longer exposure to the target dose than what has previously been expected to be tolerable. By applying the step dosing according to the present invention, i.e. applying at least two dosage steps resulting in at least three increasing dosages, the patient can be exposed to the target dosage for a prolonged period of time, such as a maximum of 52 days. Said maximum period of time results from the first and the second step lasting for two days, respectively, and the third step lasting 24 days of the remaining first treatment cycle and another 28 days of target dosage of a subsequent (second) treatment cycle which comprises only the third dosage without previous step dosing. Hence, at least one of the treatment-free periods between treatment cycles (i.e. when the bispecific antibody construct according to the present invention is uninterrupted administered) can be dispensable. Accordingly, the target dosage of the first concerned treatment cycle is immediately followed by the same target dosage of the subsequent, i.e. second concerned treatment cycle without interruption. Thereby, exposure of the patient to the target dosage is significantly expanded in order to fulfil the therapeutic goal to eradicate AML blasts and leukemic stem cells as a precondition for long-lasting therapeutic effect and eventually eradication of the AML disease in the affected and so-treated patient. Hence, the method according to the present invention provides a method which balances the need for a preferably long-lasting therapeutic effect, i.e. to eradicate the hematopoietic and the myeloid leukemic stem cells effective, and the avoidance or attenuation of severe and potentially therapy-ending side effects such as CRS. In particular, CRS events of the highest grade 5 (as commonly defined in the art) can be preferably avoided and CRS events of a higher grade 3 and 4 be significantly reduced in occurrence, i.e. grade 3 occurring typically in at most 10% of treated patients and grade 4 typically in at most 5% of treated patients, respectively.

In the context of the present invention, the duration of exposure of a patient to the bispecific antibody construct in one treatment cycle is longer than 14 days and can be up to 60 days, if two treatment cycles are not separated by a treatment-free period. Typically, each treatment cycle comprising at least two, preferably three dosage steps is followed by a treatment-free period to allow for patient recovery. However, when prolong target dosage exposure is required to address leukemic stem cells in addition to AML blasts, two treatment cycles are connected to each other by leaving the treatment-free period away. However, preferably not more than two treatment cycles follow each other without a treatment-free period in order to allow for sufficient patient recovery but still prolong target dosage exposure time.

When said two treatment cycles are connected, the later treatment cycle following the earlier treatment cycle is characterized by having only one dosage and no step dosing. This is facilitated by the fact that the step dosing of the earlier treatment cycle reduces the risk for side effects such as CRS (especially of higher grades 3 and 4 and highest grade 5) also for the immediately following treatment cycle (i.e. with no treatment-free period between the two connected cycles) because the treatment cycle following the earlier treatment cycle profits from the earlier treatment cycle's applied step dosing. Hence, side effect CRS of the highest grade could be avoided completely and the higher grades 3 and 4 attenuated to infrequent single digit occurrences. A treatment interruption could be avoided in the majority of treated patients and ensure continuous effective dose administration to treat high patients suffering from highly progressive r/r AML.

Hence, in the context of the present invention, at least one of the treatment cycles has to fulfil the requirements for the specific step dosing as described herein. In case only one treatment cycle is applied, said one treatment cycle comprises the step dosing. In case two treatment cycles are applied which are not separated by a treatment-free period, then it is sufficient for only the first of the two treatment cycles to fulfil the requirements of the specific at least three-step specific step dosing as described herein.

The period of exposure as referred to herein typically refers to the total exposure to all at least three different dosages applied through one treatment cycle. Typical exposure to the target dose is shorter, i.e. shortened by the duration of the first and second (and optionally third) dosage before the third or optional forth maximum (target) dosage within the treatment cycle is reached. Such exposure of the target dosage may last for, for example, 56, 55, 54, 53, 52, 51, 50, 25, 24, 23, 22, 21, 20, 19, 18, 17, 16, 15 or 14 days, which at the same time allows for full exploitation if the anti-tumor efficacy of the CD33×CD3 bispecific antibody construct (e.g. SEQ ID NO: 104) according to the present invention. Consequently, the present dosage regimen allows for a prolonged exposure of the treated patient to the target dose while minimizing the side effects during the initial phase of drug administration, such as cytokine release syndrome and symptoms thereof, by using step dosing as described herein. At the same time, the superior efficacy, which is confined by the administration schedule or dosage regimen as described herein, is preferably demonstrated by a significant reduction in tumor burden in treated patients, more preferably in partial or even complete remission or even repeated complete remissions after one treatment cycle or a plurality of treatment cycles, respectively.

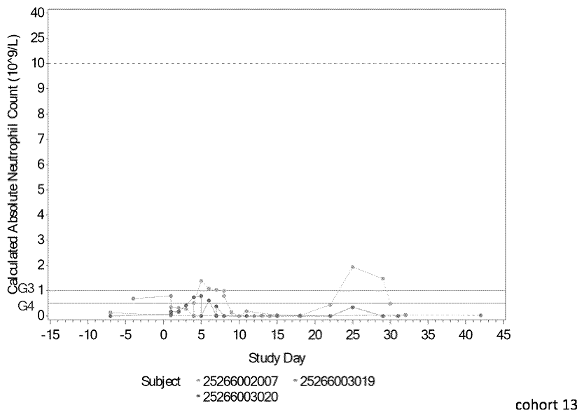

A typical treatment cycle according to the present invention, which has clinically demonstrated complete remission of disease (AML) comprises administering the CD33×CD3 bispecific antibody construct (e.g. SEQ ID NO: 104) a first dosage of 10 μg per day for two or three consecutive days, immediately followed by a second dosage of 60 μg per day for 2, 3 or 4 days, immediately followed by a third dosage of 240 μg per day for 21, 22 or 23 days, wherein the total treatment cycle duration is 28 days. Alternatively, a preferred treatment cycle comprises a first dosage of 10 μg per day for two or three consecutive days, immediately followed by a second dosage of 60 μg per day for 2, 3 or 4 days, immediately followed by a third dosage of 480 μg per day for 21, 22 or 23 days, wherein the total treatment cycle duration is 28 days. Alternatively, a preferred treatment cycle comprises a first dosage of 10 μg per day for two or three consecutive days, immediately followed by a second dosage of 60 μg per day for 2, 3 or 4 days, immediately followed by a third dosage of 600 μg per day for 21, 22 or 23 days, wherein the total treatment cycle duration is 28 days. Alternatively, a preferred treatment cycle comprises a first dosage of 10 μg per day for two or three consecutive days, immediately followed by a second dosage of 60, 120 or 240 μg per day for 2, 3 or 4 days, immediately followed by a third dosage of 720 μg per day for 21, 22 or 23 days, wherein the total treatment cycle duration is 28 days. Alternatively, a preferred treatment cycle comprises a first dosage of 10 μg per day for two or three consecutive days, immediately followed by a second dosage of 60, 120 or 240 μg per day for 2, 3 or 4 days, immediately followed by a third dosage of 840 μg per day for 21, 22 or 23 days, wherein the total treatment cycle duration is 28 days. Alternatively, a preferred treatment cycle comprises a first dosage of 10 μg per day for two or three consecutive days, immediately followed by a second dosage of 60 or 120 μg and a third dosage of 120 or 240 μg per day for together 2 days, immediately followed by a forth dosage of 840 μg per day for 21, 22 or 23 days, wherein the total treatment cycle duration is 28 days. Alternatively, a preferred treatment cycle comprises a first dosage of 10 μg per day for two or three consecutive days, immediately followed by a second dosage of 60 or 120 μg and a third dosage of 120 or 240 μg per day for together 2 days, immediately followed by a forth dosage of 960 μg per day for 21, 22 or 23 days, wherein the total treatment cycle duration is 28 days. Such treatment cycles are also represented in FIG. 5 for better illustration.

It has been a remarkable finding in the context of the present invention that already a target dosage of 240 μg per day can lead to complete remissions of disease AML, being MRD+ but preferably also MRD−. Higher target dosages such as described herein do even more quantitatively eradicate leukemic stem cells in addition to AML blasts and likely reduce the risk of relapse and thus, provide a longer disease-free state for the patient, improving their quality of life.

Preferably, in the context of the present invention, the dose toxicity limiting (DLT) window can be shortened to a standard of 4 weeks (with at least 14 days on the target dose) allowing for monitoring the onset of CRS and its resolutions, efficient intra-subject escalation, and overall patient safety.

As it is known in the art, the expression of CD33 on the surface of myeloid cells comprising the common myeloid progenitor cells, Myeloblasts, Monocytes has been demonstrated in the literature by flow cytometry. Moreover, CD33 expression on the surface of Macrophages has been demonstrated via immunohistochemistry. Those CD33⁺ cell populations in the myeloid compartment are eliminated under treatment of a patient with bispecific antibody constructs described herein. Due to the fact that some of those cell populations are itself the progenitor cells for other cell populations in the myeloid compartment the hematopoiesis of all cell types downstream of the common myeloid progenitor cells is affected which results in myelosuppression As further method of action, CD33 bispecific antibody constructs according to the present invention can also eliminate myelosuppressors (i.e. MDSC), which contribute to immune suppression within the local microenvironment.

For a successful treatment of a myeloid leukemia a significant exposure of a patient (i.e. a certain length of exposure) with the bispecific antibody constructs described herein is required to induce T cell activation/proliferation and cytotoxic activity of those T cells. However, based on the above described observations the longer the administration period of the bispecific antibody constructs last, the longer pancytopenia is to be expected. This in mind, the solution to the problem underlying this invention is to balance the length of exposure and the dose of the bispecific antibody constructs which enable the effective elimination of the leukemic cells with an off treatment period during which the myeloid compartment of a patient is allowed to recover. This is reflected by the above described administration scheme.

The time period without administration serves as recovery period for the myeloid compartment in order to rebuild myeloid cells important, e.g., for the defense against bacterial infection. The length of the required minimum time period without administration typically depends on the residual tumor burden. For example, patients who have shown a partial response, the time period may be as short as 7 days or less, such as 1, 2, 3, 4, 5, or 6 days, preferably 7 days, while those patients with higher residual tumor burden and more damage to the myeloid compartment typically require a longer period to rebuild myeloid cells, typically at least 8, 9, 10, 11, 12, 13 or 14 days, preferably 14 days. In general, it is envisaged that exposure of the patient to the target dose is maximized, and at the same time to limit the duration of a single treatment cycle including the treatment free recovery period as much as possible to allow for overall quick sequence of treatment cycles for patients who often are in a critical condition and typically need quick efficacy.

In a particular embodiment of the present invention, a first treatment cycle comprising an administration time of more than 14 days, offers a longer exposure of the patient to the target dose and thereby reduces the tumor burden to such a level that subsequent treatment cycles may not require administration times of more than 14 days. In such a case, treatment cycles after the first treatment cycle may last at most 14 days which reduces the risk of side effects by longer treatment to recovery time ratio within one cycle, provided sufficient efficiency has been reached. Alternatively, the second, third, fourth or any subsequent treatment cycle may last more than 14 days followed by one or more treatment cycles of at most 14 days in length. Also, treatment cycles of more than 14 days of administration may alternate with treatment cycles of at most 14 days of administration in order to level efficacy and mitigation of side effects. It is a specific achievement of the invention as described herein to provide a dosage scheme which does not waste time to reach an effective dose to target cancer cells and at the same time reduces the risk of triggering severe side effects such as CRS. Wasting time would not be in the interest of the treated patient who is suffering from a severe and aggressively progredient disease. On the other hand, triggering side effects such as CRS by too quick step dosing likely leads to the interruption or abandonment of treatment due to undue toxicity. Both disadvantages are mitigated by the method according to the present invention. Also, by adding a forth step, the gap between the dosages is reduced whereby also the probability of CRS is reduced. Hence, in the context of the present invention, where a high target dosage of, for example, at least 480, 720 or 960 μg per day is applied, a step dosing comprising four steps (e.g. 30-240-600-900 μg per day) is be preferred to a step dosing comprising three steps (e.g. 30-240-900 μg per day), even if conducted over the same time period due to a smaller gap between the dosage of a previous step and a target dosage.

The method according to the present invention avoids or attenuates severe side effects such as CRS. In particular, CRS events of the highest grade 5 (as commonly defined in the art) can be preferably avoided and CRS events of a higher grade 3 and 4 be significantly reduced in occurrence, i.e. grade 3 occurring typically in at most 10% of treated patients and grade 4 typically in at most 5% of treated patients undergoing a method as described herein, respectively.

As the person skilled in the art is aware of, after each relapse, a new complete remission is more and more difficult to achieve. Given the fact that patients undergoing presently described therapy by the bispecific antibody construct have typically already undergone standard chemotherapy and might have gone through remissions and relapses, a profound activity needs to be conferred by the method according to the present invention. Hence, prolonged exposure to a high dosage of bispecific antibody construct, e.g. SEQ ID NO 104, is preferred as described herein. This typically requires a step dosing comprising three dosing steps meaning 4 different and ascending dosages, i.e. a first, second, third and forth dose. Hence, such a preferred treatment cycle comprises a first dosage of 10 μg per day for two or three consecutive days, immediately followed by a second dosage of 60 or 120 μg and a third dosage of 120 or 240 μg per day for together 2 days, immediately followed by a forth dosage of 840 μg per day for 21, 22 or 23 days, wherein the total treatment cycle duration is 28 days. Alternatively, a preferred treatment cycle comprises a first dosage of 10 μg per day for two or three consecutive days, immediately followed by a second dosage of 60 or 120 μg and a third dosage of 120 or 240 μg per day for together 2 days, immediately followed by a forth dosage of 960 μg per day for 21, 22 or 23 days, wherein the total treatment cycle duration is 28 days. When two treatment cycles are combined and following each other without an intermittent administration-free period, the application of the forth, effective dosage has a duration of up to 52 days. Suh parameters are, for example, considered a prolonged exposure to a high dosage of bispecific antibody construct, e.g. SEQ ID NO 104, is preferred as described herein

The end of the period of administration is understood to be reached, when the serum level of the active compound, e.g. the bispecific compound drops under a defined threshold. An example for such threshold is a serum level below an EC₉₀ value, preferably below an EC₅₀ value, more preferably below an EC₁₀ value. Such EC values can be defined in a cytotoxic assay using CD33⁺ target cells and human PBL as effector cells in line with the assays.

In case of a bispecific single chain antibody construct such as a preferred CD33×CD3 bispecific antibody construct in the context of the present invention (see SEQ ID NO: 104), which is known to have a short serum half-life the half-life of CD33×CD3 bispecific antibody construct in mice is 6.5 to 8.7 h, while the predicted half-life of CD33×CD3 bispecific antibody construct in human is about 2 hours), the serum level would fall below the above discussed threshold value within short time after stopping a continuous iv administration, i.e. almost immediately after the end of the administration phase.

An assay for the determination of a specific ECₓ value of a bispecific antibody construct suitable for the present invention is described in the examples herein below.

The term “dose” is understood herein as a measured quantity of the agents described herein, i.e. a bispecific antibody construct, typically in units of mass such as microgram [μg].

The term “dosage” is understood herein as the rate of application of a dose of the agents described herein, i.e. a bispecific antibody construct, typically in units of mass per time such as microgram per day [μg/d]. In the context of the present invention, the application is IV infusion, preferably continuous IV infusion (CIV). Therein, administration, i.e. submission of the therapeutic bispecific antibody construct, is not interrupted during the provided period of administration.

The term “treatment cycle” is understood herein as a period of treatment, comprising at least two dosage steps resulting in at least three dosages to be applied, wherein the dosages are increasing by order of their sequence. Said dosages within one treatment cycle are preferably not interrupted by any treatment-free period between the different dosages administered within one treatment cycle applying step dosing as described herein. Instead, the continuous infusion continues with respect to the treated patient preferably uninterrupted for the entire length of the treatment cycle. After competition, said treatment cycle may then typically be followed by a period of rest (administration-free period, i.e. no treatment), and that combination of treatment period and treatment-free period is repeated on a regular schedule. For example, treatment given for four weeks followed by two weeks of rest is one treatment cycle. When this cycle is repeated multiple times on a regular schedule, it makes up a course of treatment.

The term “step dosing” is understood herein as the application of a series of increasing dosages, preferably within one treatment cycle, in order to avoid treatment-associated side effects such as CRS.

The term “dosage step” is understood herein as the change from one dosage to another. Hence, if the step dosing provides three different dosages, two dosage steps have to be applied, i.e. the change from the first to the second dosage step and from the second to the third dosage step, respectively.

In the context of the present invention, remission is understood either as the reduction or disappearance of the signs and symptoms of the disease AML. The term may also be used to refer to the period during which this diminution occurs. Herein, a remission may be considered a partial remission or a complete remission. For example, a partial remission for AML may be defined as a 50% or greater reduction in the measurable parameters of AML as may be found, for example, on physical examination, radiologic study, or by biomarker levels from a blood or urine test.

In the context of the present invention, complete remission, is typically a total disappearance of the manifestations of a disease. A patient whose condition is in complete remission might be considered cured or recovered, notwithstanding the possibility of a relapse, i.e. the reappearance of a disease.

In the context of the present invention, complete remission (CR) without a number typically means a first CR e.g. a newly diagnosed patient with AML receives chemotherapy in one or more cycles—i.e. before receiving a bispecific antibody construct according to the present invention, and goes into remission, that is the first CR (usually only called CR), then relapses, receives some other therapy and goes into remission again, that is now the second complete remission (CR2) and so forth.

The term “cohort” is understood in the context of the present invention as a group of patients who share a defining characteristic, i.e. who undergo the same treatment cycles characterized by same step dosing, dosages and application duration.

The term “effective dosage” is the target dose of at which the AML blasts and leukemic stem cells are effectively killed. This dose is typically the highest and preferably last dosage of one treatment cycle.

The term “bispecific antibody construct” refers to a molecule having a structure suitable for the specific binding of two individual target structures. In the context of the present invention such bispecific antibody constructs specifically bind to a target, preferably CD33 on the cell surface of target cells and an effector, preferably CD3 on the cell surface of T cells. However, the preferred administration as described herein, i.e. a step dosing to mitigate side effects such as a cytokine release syndrome, and a prolonged exposition to maximize efficacy, applies also to other bispecific antibody constructs targeting another target than CD33 in addition to CD3 on the cell surface of T cells. In a preferred embodiment of a bispecific antibody construct at least one, more preferably both binding domains of the bispecific antibody construct are is/are based on the structure and/or function of an antibody. Such constructs may be designated as “bispecific antibody constructs” in line with the present invention.

The term “antibody construct” refers to a molecule in which the structure and/or function is/are based on the structure and/or function of an antibody, e.g., of a full-length or whole immunoglobulin molecule. An antibody construct is hence capable of binding to its specific target or antigen and/or is/are drawn from the variable heavy chain (VH) and/or variable light chain (VL) domains of an antibody or fragment thereof. Furthermore, the domain which binds to its binding partner according to the present invention is understood herein as a binding domain of an antibody construct according to the invention. Typically, a binding domain according to the present invention comprises the minimum structural requirements of an antibody which allow for the target binding. This minimum requirement may e.g. be defined by the presence of at least the three light chain CDRs (i.e. CDR1, CDR2 and CDR3 of the VL region) and/or the three heavy chain CDRs (i.e. CDR1, CDR2 and CDR3 of the VH region), preferably of all six CDRs.

An alternative approach to define the minimal structure requirements of an antibody is the definition of the epitope of the antibody within the structure of the specific target, respectively, the protein domain of the target protein composing the epitope region (epitope cluster) or by reference to an specific antibody competing with the epitope of the defined antibody. The antibodies on which the constructs according to the invention are based include for example monoclonal, recombinant, chimeric, deimmunized, humanized and human antibodies.

The binding domain of an antibody construct according to the invention may e.g. comprise the above referred groups of CDRs. Preferably, those CDRs are comprised in the framework of an antibody light chain variable region (VL) and an antibody heavy chain variable region (VH); however, it does not have to comprise both. Fd fragments, for example, have two VH regions and often retain some antigen-binding function of the intact antigen-binding domain. Additional examples for the format of antibody fragments, antibody variants or binding domains include (1) a Fab fragment, a monovalent fragment having the VL, VH, CL and CH1 domains; (2) a F(ab′)₂ fragment, a bivalent fragment having two Fab fragments linked by a disulfide bridge at the hinge region; (3) an Fd fragment having the two VH and CH1 domains; (4) an Fv fragment having the VL and VH domains of a single arm of an antibody, (5) a dAb fragment (Ward et al., (1989) Nature 341:544-546), which has a VH domain; (6) an isolated complementarity determining region (CDR), and (7) a single chain Fv (scFv), the latter being preferred (for example, derived from an scFV-library). Examples for embodiments of antibody constructs according to the invention are e.g. described in WO 00/006605, WO 2005/040220, WO 2008/119567, WO 2010/037838, WO 2013/026837, WO 2013/026833, US 2014/0308285, US 2014/0302037, W 02014/144722, WO 2014/151910, and WO 2015/048272.

Furthermore, the definition of the term “antibody constructs” includes monovalent, bivalent and polyvalent/multivalent constructs and, thus, monospecific constructs, specifically binding to only one antigenic structure, as well as bispecific and polyspecific/multispecific constructs, which specifically bind more than one antigenic structure, e.g. two, three or more, through distinct binding domains. Moreover, the definition of the term “antibody constructs” includes molecules consisting of only one polypeptide chain as well as molecules consisting of more than one polypeptide chain, which chains can be either identical (homodimers, homotrimers or homo oligomers) or different (heterodimer, heterotrimer or heterooligomer). Examples for the above identified antibodies and variants or derivatives thereof are described inter alia in Harlow and Lane, Antibodies a laboratory manual, CSHL Press (1988) and Using Antibodies: a laboratory manual, CSHL Press (1999), Kontermann and Dübel, Antibody Engineering, Springer, 2nd ed. 2010 and Little, Recombinant Antibodies for Immunotherapy, Cambridge University Press 2009.

The antibody constructs of the present invention are preferably “in vitro generated antibody constructs”. This term refers to an antibody construct according to the above definition where all or part of the variable region (e.g., at least one CDR) is generated in a non-immune cell selection, e.g., an in vitro phage display, protein chip or any other method in which candidate sequences can be tested for their ability to bind to an antigen. This term thus preferably excludes sequences generated solely by genomic rearrangement in an immune cell in an animal. A “recombinant antibody” is an antibody made through the use of recombinant DNA technology or genetic engineering.

An embodiment of the bispecific antibody construct of the present invention is a “single chain antibody constructs”. Those single chain antibody constructs include only above described embodiments of antibody constructs, which consist of a single peptide chain.

The term “monoclonal antibody” (mAb) or monoclonal antibody construct as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site or determinant on the antigen, in contrast to conventional (polyclonal) antibody preparations which typically include different antibodies directed against different determinants (or epitopes). In addition to their specificity, the monoclonal antibodies are advantageous in that they are synthesized by the hybridoma culture, hence uncontaminated by other immunoglobulins. The modifier “monoclonal” indicates the character of the antibody as being obtained from a substantially homogeneous population of antibodies, and is not to be construed as requiring production of the antibody by any particular method.

For the preparation of monoclonal antibodies, any technique providing antibodies produced by continuous cell line cultures can be used. For example, monoclonal antibodies to be used may be made by the hybridoma method first described by Koehler et al., Nature, 256: 495 (1975), or may be made by recombinant DNA methods (see, e.g., U.S. Pat. No. 4,816,567). Examples for further techniques to produce human monoclonal antibodies include the trioma technique, the human B-cell hybridoma technique (Kozbor, Immunology Today 4 (1983), 72) and the EBV-hybridoma technique (Cole et al., Monoclonal Antibodies and Cancer Therapy, Alan R. Liss, Inc. (1985), 77-96).

Hybridomas can then be screened using standard methods, such as enzyme-linked immunosorbent assay (ELISA) and surface plasmon resonance (BIACORE™) analysis, to identify one or more hybridomas that produce an antibody that specifically binds with a specified antigen. Any form of the relevant antigen may be used as the immunogen, e.g., recombinant antigen, naturally occurring forms, any variants or fragments thereof, as well as an antigenic peptide thereof. Surface plasmon resonance as employed in the BIAcore system can be used to increase the efficiency of phage antibodies which bind to an epitope of a target antigen, such as the target cell surface antigen CD33 or CD3 epsilon (Schier, Human Antibodies Hybridomas 7 (1996), 97-105; Malmborg, J. Immunol. Methods 183 (1995), 7-13).

Another exemplary method of making monoclonal antibodies includes screening protein expression libraries, e.g., phage display or ribosome display libraries. Phage display is described, for example, in Ladner et al., U.S. Pat. No. 5,223,409; Smith (1985) Science 228:1315-1317, Clackson et al., Nature, 352: 624-628 (1991) and Marks et al., J. Mol. Biol., 222: 581-597 (1991).

In addition to the use of display libraries, the relevant antigen can be used to immunize a non-human animal, e.g., a rodent (such as a mouse, hamster, rabbit or rat). In one embodiment, the non-human animal includes at least a part of a human immunoglobulin gene. For example, it is possible to engineer mouse strains deficient in mouse antibody production with large fragments of the human Ig (immunoglobulin) loci. Using the hybridoma technology, antigen-specific monoclonal antibodies derived from the genes with the desired specificity may be produced and selected. See, e.g., XENOMOUSE™, Green et al. (1994) Nature Genetics 7:13-21, US 2003-0070185, WO 96/34096, and WO96/33735.

A monoclonal antibody can also be obtained from a non-human animal, and then modified, e.g., humanized, deimmunized, rendered chimeric etc., using recombinant DNA techniques known in the art. Examples of modified antibody constructs include humanized variants of non-human antibodies, “affinity matured” antibodies (see, e.g. Hawkins et al. J. Mol. Biol. 254, 889-896 (1992) and Lowman et al., Biochemistry 30, 10832-10837 (1991)) and antibody mutants with altered effector function(s) (see, e.g., U.S. Pat. No. 5,648,260, Kontermann and Dubel (2010), loc. cit. and Little (2009), loc. cit.).

In immunology, affinity maturation is the process by which B cells produce antibodies with increased affinity for antigen during the course of an immune response. With repeated exposures to the same antigen, a host will produce antibodies of successively greater affinities. Like the natural prototype, the in vitro affinity maturation is based on the principles of mutation and selection. The in vitro affinity maturation has successfully been used to optimize antibodies, antibody constructs, and antibody fragments. Random mutations inside the CDRs are introduced using radiation, chemical mutagens or error-prone PCR. In addition, the genetic diversity can be increased by chain shuffling. Two or three rounds of mutation and selection using display methods like phage display usually results in antibody fragments with affinities in the low nanomolar range.

A preferred type of an amino acid substitutional variation of the antibody constructs involves substituting one or more hypervariable region residues of a parent antibody (e. g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e. g. 6-7 sites) are mutated to generate all possible amino acid substitutions at each site. The antibody variants thus generated are displayed in a monovalent fashion from filamentous phage particles as fusions to the gene III product of M13 packaged within each particle. The phage-displayed variants are then screened for their biological activity (e. g. binding affinity) as herein disclosed.

In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the binding domain and, e.g., human the target cell surface antigen CD33. Such contact residues and neighboring residues are candidates for substitution according to the techniques elaborated herein. Once such variants are generated, the panel of variants is subjected to screening as described herein and antibodies with superior properties in one or more relevant assays may be selected for further development.

The monoclonal antibodies and antibody constructs of the present invention specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81: 6851-6855 (1984)). Chimeric antibodies of interest herein include “primitized” antibodies comprising variable domain antigen-binding sequences derived from a non-human primate (e.g., Old World Monkey, Ape etc.) and human constant region sequences. A variety of approaches for making chimeric antibodies have been described. See e.g., Morrison et al., Proc. Natl. Acad. ScL U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., EP 0171496; EP 0173494; and GB 2177096.

An antibody, antibody construct or antibody fragment may also be modified by specific deletion of human T cell epitopes (a method called “deimmunization”) by the methods disclosed in WO 98/52976 and WO 00/34317. Briefly, the heavy and light chain variable domains of an antibody can be analyzed for peptides that bind to MHC class II; these peptides represent potential T cell epitopes (as defined in WO 98/52976 and WO 00/34317). For detection of potential T cell epitopes, a computer modeling approach termed “peptide threading” can be applied, and in addition a database of human MHC class II binding peptides can be searched for motifs present in the VH and VL sequences, as described in WO 98/52976 and WO 00/34317. These motifs bind to any of the 18 major MHC class II DR allotypes, and thus constitute potential T cell epitopes. Potential T cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. Human germline sequences are disclosed e.g. in Tomlinson, et al. (1992) J. Mol. Biol. 227:776-798; Cook, G. P. et al. (1995) Immunol. Today Vol. 16 (5): 237-242; and Tomlinson et al. (1995) EMBO J. 14: 14:4628-4638. The V BASE directory provides a comprehensive directory of human immunoglobulin variable region sequences (compiled by Tomlinson, L A. et al. MRC Centre for Protein Engineering, Cambridge, UK). These sequences can be used as a source of human sequence, e.g., for framework regions and CDRs. Consensus human framework regions can also be used, for example as described in U.S. Pat. No. 6,300,064.

“Humanized” antibodies, antibody constructs or fragments thereof (such as Fv, Fab, Fab′, F(ab′)₂ or other antigen-binding subsequences of antibodies) are antibodies or immunoglobulins of mostly human sequences, which contain (a) minimal sequence(s) derived from non-human immunoglobulin. For the most part, humanized antibodies are human immunoglobulins (recipient antibody) in which residues from a hypervariable region (also CDR) of the recipient are replaced by residues from a hypervariable region of a non-human (e.g., rodent) species (donor antibody) such as mouse, rat, hamster or rabbit having the desired specificity, affinity, and capacity. In some instances, Fv framework region (FR) residues of the human immunoglobulin are replaced by corresponding non-human residues. Furthermore, “humanized antibodies” as used herein may also comprise residues which are found neither in the recipient antibody nor the donor antibody. These modifications are made to further refine and optimize antibody performance. The humanized antibody may also comprise at least a portion of an immunoglobulin constant region (Fc), typically that of a human immunoglobulin. For further details, see Jones et al., Nature, 321: 522-525 (1986); Reichmann et al., Nature, 332: 323-329 (1988); and Presta, Curr. Op. Struct. Biol., 2: 593-596 (1992).

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by Morrison (1985) Science 229:1202-1207; by Oi et al. (1986) BioTechniques 4:214; and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

Humanized antibodies may also be produced using transgenic animals such as mice that express human heavy and light chain genes, but are incapable of expressing the endogenous mouse immunoglobulin heavy and light chain genes. Winter describes an exemplary CDR grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

A humanized antibody can be optimized by the introduction of conservative substitutions, consensus sequence substitutions, germline substitutions and/or back mutations. Such altered immunoglobulin molecules can be made by any of several techniques known in the art, (e.g., Teng et al., Proc. Natl. Acad. Sci. U.S.A., 80: 7308-7312, 1983; Kozbor et al., Immunology Today, 4: 7279, 1983; Olsson et al., Meth. Enzymol., 92: 3-16, 1982, and EP 239 400.

The term “human antibody”, “human antibody construct” and “human binding domain” includes antibodies, antibody constructs and binding domains having antibody regions such as variable and constant regions or domains which correspond substantially to human germline immunoglobulin sequences known in the art, including, for example, those described by Kabat et al. (1991) (loc. cit.). The human antibodies, antibody constructs or binding domains of the invention may include amino acid residues not encoded by human germline immunoglobulin sequences (e.g., mutations introduced by random or site-specific mutagenesis in vitro or by somatic mutation in vivo), for example in the CDRs, and in particular, in CDR3. The human antibodies, antibody constructs or binding domains can have at least one, two, three, four, five, or more positions replaced with an amino acid residue that is not encoded by the human germline immunoglobulin sequence. The definition of human antibodies, antibody constructs and binding domains as used herein also contemplates fully human antibodies, which include only non-artificially and/or genetically altered human sequences of antibodies as those can be derived by using technologies or systems such as the Xenomouse.

In some embodiments, the antibody constructs of the invention are “isolated” or “substantially pure” antibody constructs. “Isolated” or “substantially pure” when used to describe the antibody construct disclosed herein means an antibody construct that has been identified, separated and/or recovered from a component of its production environment. Preferably, the antibody construct is free or substantially free of association with all other components from its production environment. Contaminant components of its production environment, such as that resulting from recombinant transfected cells, are materials that would typically interfere with diagnostic or therapeutic uses for the polypeptide, and may include enzymes, hormones, and other proteinaceous or non-proteinaceous solutes. The antibody constructs may e.g constitute at least about 5%, or at least about 50% by weight of the total protein in a given sample. It is understood that the isolated protein may constitute from 5% to 99.9% by weight of the total protein content, depending on the circumstances. The polypeptide may be made at a significantly higher concentration through the use of an inducible promoter or high expression promoter, such that it is made at increased concentration levels. The definition includes the production of an antibody construct in a wide variety of organisms and/or host cells that are known in the art. In preferred embodiments, the antibody construct will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Ordinarily, however, an isolated antibody construct will be prepared by at least one purification step.

The term “binding domain” characterizes in connection with the present invention a domain which (specifically) binds to/interacts with/recognizes a given target epitope or a given target site on the target molecules (antigens) and CD3, respectively. The structure and function of the first binding domain (recognizing the target cell surface antigen CD33), and preferably also the structure and/or function of the second binding domain (CD3), is/are based on the structure and/or function of an antibody, e.g. of a full-length or whole immunoglobulin molecule. According to the invention, the first binding domain is characterized by the presence of three light chain CDRs (i.e. CDR1, CDR2 and CDR3 of the VL region) and three heavy chain CDRs (i.e. CDR1, CDR2 and CDR3 of the VH region). The second binding domain preferably also comprises the minimum structural requirements of an antibody which allow for the target binding. More preferably, the second binding domain comprises at least three light chain CDRs (i.e. CDR1, CDR2 and CDR3 of the VL region) and/or three heavy chain CDRs (i.e. CDR1, CDR2 and CDR3 of the VH region). It is envisaged that the first and/or second binding domain is produced by or obtainable by phage-display or library screening methods rather than by grafting CDR sequences from a pre-existing (monoclonal) antibody into a scaffold.

According to the present invention, binding domains are preferably in the form of polypeptides. Such polypeptides may include proteinaceous parts and non-proteinaceous parts (e.g. chemical linkers or chemical cross-linking agents such as glutaraldehyde). Proteins (including fragments thereof, preferably biologically active fragments, and peptides, usually having less than 30 amino acids) comprise two or more amino acids coupled to each other via a covalent peptide bond (resulting in a chain of amino acids). The term “polypeptide” as used herein describes a group of molecules, which usually consist of more than 30 amino acids. Polypeptides may further form multimers such as dimers, trimers and higher oligomers, i.e. consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a hereteromultimer is an antibody molecule, which, in its naturally occurring form, consists of two identical light polypeptide chains and two identical heavy polypeptide chains. The terms “peptide”, “polypeptide” and “protein” also refer to naturally modified peptides/polypeptides/proteins wherein the modification is effected e.g. by post-translational modifications like glycosylation, acetylation, phosphorylation and the like. A “peptide”, “polypeptide” or “protein” when referred to herein may also be chemically modified such as pegylated. Such modifications are well known in the art and described herein below.

Antibodies and antibody constructs comprising at least one human binding domain avoid some of the problems associated with antibodies or antibody constructs that possess non-human such as rodent (e.g. murine, rat, hamster or rabbit) variable and/or constant regions. The presence of such rodent derived proteins can lead to the rapid clearance of the antibodies or antibody constructs or can lead to the generation of an immune response against the antibody or antibody construct by a patient. In order to avoid the use of rodent derived antibodies or antibody constructs, human or fully human antibodies/antibody constructs can be generated through the introduction of human antibody function into a rodent so that the rodent produces fully human antibodies.

The ability to clone and reconstruct megabase-sized human loci in YACs and to introduce them into the mouse germline provides a powerful approach to elucidating the functional components of very large or crudely mapped loci as well as generating useful models of human disease. Furthermore, the use of such technology for substitution of mouse loci with their human equivalents could provide unique insights into the expression and regulation of human gene products during development, their communication with other systems, and their involvement in disease induction and progression.

An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies as well as their role in B-cell development. Furthermore, such a strategy could provide an ideal source for production of fully human monoclonal antibodies (mAbs)—an important milestone towards fulfilling the promise of antibody therapy in human disease. Fully human antibodies or antibody constructs are expected to minimize the immunogenic and allergic responses intrinsic to mouse or mouse-derivatized mAbs and thus to increase the efficacy and safety of the administered antibodies/antibody constructs. The use of fully human antibodies or antibody constructs can be expected to provide a substantial advantage in the treatment of chronic and recurring human diseases, such as inflammation, autoimmunity, and cancer, which require repeated compound administrations.

One approach towards this goal was to engineer mouse strains deficient in mouse antibody production with large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human mAbs with the desired specificity could be readily produced and selected. This general strategy was demonstrated in connection with the generation of the first XenoMouse mouse strains (see Green et al. Nature Genetics 7:13-21 (1994)). The XenoMouse strains were engineered with yeast artificial chromosomes (YACs) containing 245 kb and 190 kb-sized germline configuration fragments of the human heavy chain locus and kappa light chain locus, respectively, which contained core variable and constant region sequences. The human Ig containing YACs proved to be compatible with the mouse system for both rearrangement and expression of antibodies and were capable of substituting for the inactivated mouse Ig genes. This was demonstrated by their ability to induce B cell development, to produce an adult-like human repertoire of fully human antibodies, and to generate antigen-specific human mAbs. These results also suggested that introduction of larger portions of the human Ig loci containing greater numbers of V genes, additional regulatory elements, and human Ig constant regions might recapitulate substantially the full repertoire that is characteristic of the human humoral response to infection and immunization. The work of Green et al. was recently extended to the introduction of greater than approximately 80% of the human antibody repertoire through introduction of megabase sized, germline configuration YAC fragments of the human heavy chain loci and kappa light chain loci, respectively. See Mendez et al. Nature Genetics 15:146-156 (1997) and U.S. patent application Ser. No. 08/759,620.

The production of the XenoMouse mice is further discussed and delineated in U.S. patent application Ser. No. 07/466,008, Ser. No. 07/610,515, Ser. No. 07/919,297, Ser. No. 07/922,649, Ser. No. 08/031,801, Ser. No. 08/112,848, Ser. No. 08/234,145, Ser. No. 08/376,279, Ser. No. 08/430,938, Ser. No. 08/464,584, Ser. No. 08/464,582, Ser. No. 08/463,191, Ser. No. 08/462,837, Ser. No. 08/486,853, Ser. No. 08/486,857, Ser. No. 08/486,859, Ser. No. 08/462,513, Ser. No. 08/724,752, and Ser. No. 08/759,620; and U.S. Pat. Nos. 6,162,963, 6,150,584, 6,114,598, 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med. 188:483-495 (1998), EP 0 463 151 B1, WO 94/02602, WO 96/34096, WO 98/24893, WO 00/76310, and WO 03/47336.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more VH genes, one or more DH genes, one or more JH genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806, 5,625,825, 5,625,126, 5,633,425, 5,661,016, 5,770,429, 5,789,650, 5,814,318, 5,877,397, 5,874,299, and 6,255,458 each to Lonberg and Kay, U.S. Pat. Nos. 5,591,669 and 6,023,010 to Krimpenfort and Berns, U.S. Pat. Nos. 5,612,205, 5,721,367, and 5,789,215 to Berns et al., and U.S. Pat. No. 5,643,763 to Choi and Dunn, and GenPharm International U.S. patent application Ser. No. 07/574,748, Ser. No. 07/575,962, Ser. No. 07/810,279, Ser. No. 07/853,408, Ser. No. 07/904,068, Ser. No. 07/990,860, Ser. No. 08/053,131, Ser. No. 08/096,762, Ser. No. 08/155,301, Ser. No. 08/161,739, Ser. No. 08/165,699, Ser. No. 08/209,741. See also EP 0 546 073 B1, WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175. See further Taylor et al. (1992), Chen et al. (1993), Tuaillon et al. (1993), Choi et al. (1993), Lonberg et al. (1994), Taylor et al. (1994), and Tuaillon et al. (1995), Fishwild et al. (1996).

Kirin has also demonstrated the generation of human antibodies from mice in which, through microcell fusion, large pieces of chromosomes, or entire chromosomes, have been introduced. See European Patent Application Nos. 773 288 and 843 961. Xenerex Biosciences is developing a technology for the potential generation of human antibodies. In this technology, SCID mice are reconstituted with human lymphatic cells, e.g., B and/or T cells. Mice are then immunized with an antigen and can generate an immune response against the antigen. See U.S. Pat. Nos. 5,476,996; 5,698,767; and 5,958,765.

Human anti-mouse antibody (HAMA) responses have led the industry to prepare chimeric or otherwise humanized antibodies. It is however expected that certain human anti-chimeric antibody (HACA) responses will be observed, particularly in chronic or multi-dose utilizations of the antibody. Thus, it would be desirable to provide antibody constructs comprising a fully human binding domain against the target cell surface antigen and a fully human binding domain against CD3 in order to vitiate concerns and/or effects of HAMA or HACA response.

The terms “(specifically) binds to”, (specifically) recognizes”, “is (specifically) directed to”, and “(specifically) reacts with” mean in accordance with this invention that a binding domain interacts or specifically interacts with one or more, preferably at least two, more preferably at least three and most preferably at least four amino acids of an epitope located on the target protein or antigen (the target cell surface antigen CD33/CD3).

The term “epitope” refers to the site on an antigen to which a binding domain, such as an antibody or immunoglobulin or derivative or fragment of an antibody or of an immunoglobulin, specifically binds. An “epitope” is antigenic and thus the term epitope is sometimes also referred to herein as “antigenic structure” or “antigenic determinant”. Thus, the binding domain is an “antigen interaction site”. Said binding/interaction is also understood to define a “specific recognition”. The term “epitope” is understood in connection with this application as describing the complete antigenic structure, whereas the term “part of the epitope” may be used to describe one or more subgroups of the specific epitope of a given binding domain.

“Epitopes” can be formed both by contiguous amino acids or non-contiguous amino acids juxtaposed by tertiary folding of a protein. A “linear epitope” is an epitope where an amino acid primary sequence comprises the recognized epitope. A linear epitope typically includes at least 3 or at least 4, and more usually, at least 5 or at least 6 or at least 7, for example, about 8 to about 10 amino acids in a unique sequence.

A “conformational epitope”, in contrast to a linear epitope, is an epitope wherein the primary sequence of the amino acids comprising the epitope is not the sole defining component of the epitope recognized (e.g., an epitope wherein the primary sequence of amino acids is not necessarily recognized by the binding domain). Typically a conformational epitope comprises an increased number of amino acids relative to a linear epitope. With regard to recognition of conformational epitopes, the binding domain recognizes a three-dimensional structure of the antigen, preferably a peptide or protein or fragment thereof (in the context of the present invention, the antigen for one of the binding domains is comprised within the target cell surface antigen CD33). For example, when a protein molecule folds to form a three-dimensional structure, certain amino acids and/or the polypeptide backbone forming the conformational epitope become juxtaposed enabling the antibody to recognize the epitope. Methods of determining the conformation of epitopes include, but are not limited to, x-ray crystallography, two-dimensional nuclear magnetic resonance (2D-NMR) spectroscopy and site-directed spin labelling and electron paramagnetic resonance (EPR) spectroscopy.

The interaction between the binding domain and the epitope or epitope cluster implies that a binding domain exhibits appreciable affinity for the epitope or epitope cluster on a particular protein or antigen (here: the target cell surface antigen CD33 and CD3, respectively) and, generally, does not exhibit significant reactivity with proteins or antigens other than the target cell surface antigen CD33 or CD3. “Appreciable affinity” includes binding with an affinity of about 10⁻⁶ M (KD) or stronger. Preferably, binding is considered specific when the binding affinity is about 10⁻¹² to 10⁻⁸ M, 10⁻¹² to 10⁻⁹ M, 10⁻¹² to 10⁻¹⁰ M, 10⁻¹¹ to 10⁻⁸ M, preferably of about 10⁻¹¹ to 10⁻⁹ M. Whether a binding domain specifically reacts with or binds to a target can be tested readily by, inter alia, comparing the reaction of said binding domain with a target protein or antigen with the reaction of said binding domain with proteins or antigens other than the target cell surface antigen CD33 or CD3. Preferably, a binding domain of the invention does not essentially or substantially bind to proteins or antigens other than the target cell surface antigen CD33 or CD3 (i.e., the first binding domain is not capable of binding to proteins other than the target cell surface antigen CD33 and the second binding domain is not capable of binding to proteins other than CD3).

The term “does not essentially/substantially bind” or “is not capable of binding” means that a binding domain of the present invention does not bind a protein or antigen other than the target cell surface antigen CD33 or CD3, i.e., does not show reactivity of more than 30%, preferably not more than 20%, more preferably not more than 10%, particularly preferably not more than 9%, 8%, 7%, 6% or 5% with proteins or antigens other than the target cell surface antigen CD33 or CD3, whereby binding to the target cell surface antigen CD33 or CD3, respectively, is set to be 100%.

Specific binding is believed to be effected by specific motifs in the amino acid sequence of the binding domain and the antigen. Thus, binding is achieved as a result of their primary, secondary and/or tertiary structure as well as the result of secondary modifications of said structures. The specific interaction of the antigen-interaction-site with its specific antigen may result in a simple binding of said site to the antigen. Moreover, the specific interaction of the antigen-interaction-site with its specific antigen may alternatively or additionally result in the initiation of a signal, e.g. due to the induction of a change of the conformation of the antigen, an oligomerization of the antigen, etc.

The term “variable” refers to the portions of the antibody or immunoglobulin domains that exhibit variability in their sequence and that are involved in determining the specificity and binding affinity of a particular antibody (i.e., the “variable domain(s)”). The pairing of a variable heavy chain (VH) and a variable light chain (VL) together forms a single antigen-binding site.

Variability is not evenly distributed throughout the variable domains of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable domains are called the “framework” regions (FRM) and provide a scaffold for the six CDRs in three dimensional space to form an antigen-binding surface. The variable domains of naturally occurring heavy and light chains each comprise four FRM regions (FR1, FR2, FR3, and FR4), largely adopting a β-sheet configuration, connected by three hypervariable regions, which form loops connecting, and in some cases forming part of, the β-sheet structure. The hypervariable regions in each chain are held together in close proximity by the FRM and, with the hypervariable regions from the other chain, contribute to the formation of the antigen-binding site (see Kabat et al., loc. cit.).

The terms “CDR”, and its plural “CDRs”, refer to the complementarity determining region of which three make up the binding character of a light chain variable region (CDR-L1, CDR-L2 and CDR-L3) and three make up the binding character of a heavy chain variable region (CDR-H1, CDR-H2 and CDR-H3). CDRs contain most of the residues responsible for specific interactions of the antibody with the antigen and hence contribute to the functional activity of an antibody molecule: they are the main determinants of antigen specificity.

The exact definitional CDR boundaries and lengths are subject to different classification and numbering systems. CDRs may therefore be referred to by Kabat, Chothia, contact or any other boundary definitions, including the numbering system described herein. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat (an approach based on cross-species sequence variability), Chothia (an approach based on crystallographic studies of antigen-antibody complexes), and/or MacCallum (Kabat et al., loc. cit.; Chothia et al., J. Mol. Biol, 1987, 196: 901-917; and MacCallum et al., J. Mol. Biol, 1996, 262: 732). Still another standard for characterizing the antigen binding site is the AbM definition used by Oxford Molecular's AbM antibody modeling software. See, e.g., Protein Sequence and Structure Analysis of Antibody Variable Domains. In: Antibody Engineering Lab Manual (Ed.: Duebel, S. and Kontermann, R., Springer-Verlag, Heidelberg). To the extent that two residue identification techniques define regions of overlapping, but not identical regions, they can be combined to define a hybrid CDR. However, the numbering in accordance with the so-called Kabat system is preferred.

Typically, CDRs form a loop structure that can be classified as a canonical structure. The term “canonical structure” refers to the main chain conformation that is adopted by the antigen binding (CDR) loops. From comparative structural studies, it has been found that five of the six antigen binding loops have only a limited repertoire of available conformations. Each canonical structure can be characterized by the torsion angles of the polypeptide backbone. Correspondent loops between antibodies may, therefore, have very similar three dimensional structures, despite high amino acid sequence variability in most parts of the loops (Chothia and Lesk, J. Mol. Biol., 1987, 196: 901; Chothia et al., Nature, 1989, 342: 877; Martin and Thornton, J. Mol. Biol, 1996, 263: 800). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class can therefore be made based on the presence of these key amino acid residues.

The term “canonical structure” may also include considerations as to the linear sequence of the antibody, for example, as catalogued by Kabat (Kabat et al., loc. cit.). The Kabat numbering scheme (system) is a widely adopted standard for numbering the amino acid residues of an antibody variable domain in a consistent manner and is the preferred scheme applied in the present invention as also mentioned elsewhere herein. Additional structural considerations can also be used to determine the canonical structure of an antibody. For example, those differences not fully reflected by Kabat numbering can be described by the numbering system of Chothia et al and/or revealed by other techniques, for example, crystallography and two- or three-dimensional computational modeling. Accordingly, a given antibody sequence may be placed into a canonical class which allows for, among other things, identifying appropriate chassis sequences (e.g., based on a desire to include a variety of canonical structures in a library). Kabat numbering of antibody amino acid sequences and structural considerations as described by Chothia et al., loc. cit. and their implications for construing canonical aspects of antibody structure, are described in the literature. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988.

The CDR3 of the light chain and, particularly, the CDR3 of the heavy chain may constitute the most important determinants in antigen binding within the light and heavy chain variable regions. In some antibody constructs, the heavy chain CDR3 appears to constitute the major area of contact between the antigen and the antibody. In vitro selection schemes in which CDR3 alone is varied can be used to vary the binding properties of an antibody or determine which residues contribute to the binding of an antigen. Hence, CDR3 is typically the greatest source of molecular diversity within the antibody-binding site. H3, for example, can be as short as two amino acid residues or greater than 26 amino acids.

In a classical full-length antibody or immunoglobulin, each light (L) chain is linked to a heavy (H) chain by one covalent disulfide bond, while the two H chains are linked to each other by one or more disulfide bonds depending on the H chain isotype. The CH domain most proximal to VH is usually designated as CH1. The constant (“C”) domains are not directly involved in antigen binding, but exhibit various effector functions, such as antibody-dependent, cell-mediated cytotoxicity and complement activation. The Fc region of an antibody is comprised within the heavy chain constant domains and is for example able to interact with cell surface located Fc receptors.

The sequence of antibody genes after assembly and somatic mutation is highly varied, and these varied genes are estimated to encode 10¹⁰ different antibody molecules (Immunoglobulin Genes, 2^{nd} ed., eds. Jonio et al., Academic Press, San Diego, Calif., 1995). Accordingly, the immune system provides a repertoire of immunoglobulins. The term “repertoire” refers to at least one nucleotide sequence derived wholly or partially from at least one sequence encoding at least one immunoglobulin. The sequence(s) may be generated by rearrangement in vivo of the V, D, and J segments of heavy chains, and the V and J segments of light chains. Alternatively, the sequence(s) can be generated from a cell in response to which rearrangement occurs, e.g., in vitro stimulation. Alternatively, part or all of the sequence(s) may be obtained by DNA splicing, nucleotide synthesis, mutagenesis, and other methods, see, e.g., U.S. Pat. No. 5,565,332. A repertoire may include only one sequence or may include a plurality of sequences, including ones in a genetically diverse collection.

The term “bispecific” as used herein refers to a construct which is “at least bispecific”, i.e., it comprises at least a first binding domain and a second binding domain, wherein the first binding domain binds to one antigen or target, and the second binding domain binds to another antigen or target (here: CD3). Accordingly, bispecific antibody constructs according to the invention comprise specificities for at least two different antigens or targets. The term “bispecific antibody construct” of the invention also encompasses multispecific constructs such as trispecific constructs, the latter ones including three binding domains, or constructs having more than three (e.g. four, five . . . ) specificities. In case the construct used in connection with this invention is an antibody construct, these encompassed corresponding constructs are multispecific antibody constructs such as trispecific antibody constructs, the latter ones including three binding domains, or constructs having more than three (e.g. four, five . . . ) specificities.

Given that the antibody constructs according to the invention are (at least) bispecific, they do not occur naturally and they are markedly different from naturally occurring products. A “bispecific” antibody construct or immunoglobulin is hence an artificial hybrid antibody or immunoglobulin having at least two distinct binding sites with different specificities. Bispecific antibodies can be produced by a variety of methods including fusion of hybridomas or linking of Fab′ fragments. See, e.g., Songsivilai & Lachmann, Clin. Exp. Immunol. 79:315-321 (1990).

The at least two binding domains and the variable domains of the antibody construct of the present invention may or may not comprise peptide linkers (spacer peptides). The term “peptide linker” defines in accordance with the present invention an amino acid sequence by which the amino acid sequences of one (variable and/or binding) domain and another (variable and/or binding) domain of the antibody construct of the invention are linked with each other. An essential technical feature of such peptide linker is that said peptide linker does not comprise any polymerization activity. Among the suitable peptide linkers are those described in U.S. Pat. Nos. 4,751,180 and 4,935,233 or WO 88/09344. The peptide linkers can also be used to attach other domains or modules or regions (such as half-life extending domains) to the antibody construct of the invention.

In the event that a linker is used, this linker is preferably of a length and sequence sufficient to ensure that each of the first and second domains can, independently from one another, retain their differential binding specificities. For peptide linkers which connect the at least two binding domains in the antibody construct of the invention (or two variable domains), those peptide linkers are preferred which comprise only a few number of amino acid residues, e.g. 12 amino acid residues or less. Thus, peptide linker of 12, 11, 10, 9, 8, 7, 6 or 5 amino acid residues are preferred. An envisaged peptide linker with less than 5 amino acids comprises 4, 3, 2 or one amino acid(s) wherein Gly-rich linkers are preferred. A particularly preferred “single” amino acid in context of said “peptide linker” is Gly. Accordingly, said peptide linker may consist of the single amino acid Gly. Another preferred embodiment of a peptide linker is characterized by the amino acid sequence Gly-Gly-Gly-Gly-Ser, i.e. Gly₄Ser, or polymers thereof, i.e. (Gly₄Ser)x, where x is an integer of 1 or greater. The characteristics of said peptide linker, which comprise the absence of the promotion of secondary structures are known in the art and are described e.g. in Dall'Acqua et al. (Biochem. (1998) 37, 9266-9273), Cheadle et al. (Mol Immunol (1992) 29, 21-30) and Raag and Whitlow (FASEB (1995) 9(1), 73-80). Peptide linkers which also do not promote any secondary structures are preferred. The linkage of said domains to each other can be provided by, e.g. genetic engineering, as described in the examples. Methods for preparing fused and operatively linked bispecific single chain constructs and expressing them in mammalian cells or bacteria are well-known in the art (e.g. WO 99/54440 or Sambrook et al., Molecular Cloning: A Laboratory Manual, Cold Spring Harbor Laboratory Press, Cold Spring Harbor, N.Y., 2001).

Bispecific single chain molecules are known in the art and are described in WO 99/54440, Mack, J. Immunol. (1997), 158, 3965-3970, Mack, PNAS, (1995), 92, 7021-7025, Kufer, Cancer Immunol. Immunother., (1997), 45, 193-197, Löffler, Blood, (2000), 95, 6, 2098-2103, Brühl, Immunol., (2001), 166, 2420-2426, Kipriyanov, J. Mol. Biol., (1999), 293, 41-56. Techniques described for the production of single chain antibodies (see, inter alia, U.S. Pat. No. 4,946,778, Kontermann and Dübel (2010), loc. cit. and Little (2009), loc. cit.) can be adapted to produce single chain antibody constructs specifically recognizing (an) elected target(s).

Bivalent (also called divalent) or bispecific single-chain variable fragments (bi-scFvs or di-scFvs having the format (scFv)₂) can be engineered by linking two scFv molecules. In case these two scFv molecules have the same binding specificity, the resulting (scFv)₂ molecule will preferably be called bivalent (i.e. it has two valences for the same target epitope). In case the two scFv molecules have different binding specificities, the resulting (scFv)₂ molecule will preferably be called bispecific. The linking can be done by producing a single peptide chain with two VH regions and two VL regions, yielding tandem scFvs (see e.g. Kufer P. et al., (2004) Trends in Biotechnology 22(5):238-244). Another possibility is the creation of scFv molecules with linker peptides that are too short for the two variable regions to fold together (e.g. about five amino acids), forcing the scFvs to dimerize. This type is known as diabodies (see e.g. Hollinger, Philipp et al., (July 1993) Proceedings of the National Academy of Sciences of the United States of America 90 (14): 6444-8.).

Single domain antibodies comprise merely one (monomeric) antibody variable domain which is able to bind selectively to a specific antigen, independently of other V regions or domains. The first single domain antibodies were engineered from heavy chain antibodies found in camelids, and these are called V_{H}H fragments. Cartilaginous fishes also have heavy chain antibodies (IgNAR) from which single domain antibodies called V_{NAR} fragments can be obtained. An alternative approach is to split the dimeric variable domains from common immunoglobulins e.g. from humans or rodents into monomers, hence obtaining VH or VL as a single domain Ab. Although most research into single domain antibodies is currently based on heavy chain variable domains, nanobodies derived from light chains have also been shown to bind specifically to target epitopes. Examples of single domain antibodies are called sdAb, nanobodies or single variable domain antibodies.

A (single domain mAb)₂ is hence a monoclonal antibody construct composed of (at least) two single domain monoclonal antibodies, which are individually selected from the group comprising VH, VL, VHH and V_{NAR}. The linker is preferably in the form of a peptide linker. Similarly, an “scFv-single domain mAb” is a monoclonal antibody construct composed of at least one single domain antibody as described above and one scFv molecule as described above. Again, the linker is preferably in the form of a peptide linker.

It is also envisaged that the antibody construct of the invention has, in addition to its function to bind to the target antigen CD33 and CD3, a further function. In this format, the antibody construct is a trifunctional or multifunctional antibody construct by targeting target cells through binding to the target antigen, mediating cytotoxic T cell activity through CD3 binding and providing a further function such as a label (fluorescent etc.), a therapeutic agent such as a toxin or radionuclide, etc.

Covalent modifications of the antibody constructs are also included within the scope of this invention, and are generally, but not always, done post-translationally. For example, several types of covalent modifications of the antibody construct are introduced into the molecule by reacting specific amino acid residues of the antibody construct with an organic derivatizing agent that is capable of reacting with selected side chains or the N- or C-terminal residues.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1 M sodium cacodylate at pH 6.0. Lysinyl and amino terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using ¹²⁵I or ¹³¹I to prepare labeled proteins for use in radioimmunoassay, the chloramine T method described above being suitable.

Carboxyl side groups (aspartyl or glutamyl) are selectively modified by reaction with carbodiimides (R′—N═C═N—R), where R and R′ are optionally different alkyl groups, such as 1-cyclohexyl-3-(2-morpholinyl-4-ethyl) carbodiimide or 1-ethyl-3-(4-azonia-4,4-dimethyl pentyl) carbodiimide. Furthermore, aspartyl and glutamyl residues are converted to asparaginyl and glutaminyl residues by reaction with ammonium ions.

Derivatization with bifunctional agents is useful for crosslinking the antibody constructs of the present invention to a water-insoluble support matrix or surface for use in a variety of methods. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, 1983, pp. 79-86), acetylation of the N-terminal amine, and amidation of any C-terminal carboxyl group.

Another type of covalent modification of the antibody constructs included within the scope of this invention comprises altering the glycosylation pattern of the protein. As is known in the art, glycosylation patterns can depend on both the sequence of the protein (e.g., the presence or absence of particular glycosylation amino acid residues, discussed below), or the host cell or organism in which the protein is produced. Particular expression systems are discussed below.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Addition of glycosylation sites to the antibody construct is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for 0-linked glycosylation sites). For ease, the amino acid sequence of an antibody construct is preferably altered through changes at the DNA level, particularly by mutating the DNA encoding the polypeptide at preselected bases such that codons are generated that will translate into the desired amino acids.

Another means of increasing the number of carbohydrate moieties on the antibody construct is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330, and in Aplin and Wriston, 1981, CRC Crit. Rev. Biochem., pp. 259-306.

Removal of carbohydrate moieties present on the starting antibody construct may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118:131. Enzymatic cleavage of carbohydrate moieties on polypeptides can be achieved by the use of a variety of endo- and exo-glycosidases as described by Thotakura et al., 1987, Meth. Enzymol. 138:350. Glycosylation at potential glycosylation sites may be prevented by the use of the compound tunicamycin as described by Duskin et al., 1982, J. Biol. Chem. 257:3105. Tunicamycin blocks the formation of protein-N-glycoside linkages.

Other modifications of the antibody construct are contemplated herein. For example, another type of covalent modification of the antibody construct comprises linking the antibody construct to various non-proteinaceous polymers, including, but not limited to, various polyols such as polyethylene glycol, polypropylene glycol, polyoxyalkylenes, or copolymers of polyethylene glycol and polypropylene glycol, in the manner set forth in U.S. Pat. Nos. 4,640,835; 4,496,689; 4,301,144; 4,670,417; 4,791,192 or 4,179,337. In addition, as is known in the art, amino acid substitutions may be made in various positions within the antibody construct, e.g. in order to facilitate the addition of polymers such as PEG.

In some embodiments, the covalent modification of the antibody constructs of the invention comprises the addition of one or more labels. The labelling group may be coupled to the antibody construct via spacer arms of various lengths to reduce potential steric hindrance. Various methods for labelling proteins are known in the art and can be used in performing the present invention. The term “label” or “labelling group” refers to any detectable label. In general, labels fall into a variety of classes, depending on the assay in which they are to be detected—the following examples include, but are not limited to:
a) isotopic labels, which may be radioactive or heavy isotopes, such as radioisotopes or radionuclides (e.g., ³H, ¹⁴C, ¹⁵N, ³⁵S, ⁸⁹Zr, ⁹⁰Y, ⁹⁹Tc, ¹¹¹In, ¹²⁵I, ¹³¹I)
b) magnetic labels (e.g., magnetic particles)
c) redox active moieties
d) optical dye (including, but not limited to, chromophores, phosphors and fluorophores) such as fluorescent groups (e.g., FITC, rhodamine, lanthanide phosphors), chemilluminescent groups, and fluorophores which can be either “small molecule” fluores or proteinaceous fluores
e) enzymatic groups (e.g. horseradish peroxidase, β-galactosidase, luciferase, alkaline phosphatase)
f) biotinylated groups
g) predetermined polypeptide epitopes recognized by a secondary reporter (e.g., leucine zipper pair sequences, binding sites for secondary antibodies, metal binding domains, epitope tags, etc.)

By “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade BlueJ, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, Oreg.), FITC, Rhodamine, and Texas Red (Pierce, Rockford, Ill.), Cy5, Cy5.5, Cy7 (Amersham Life Science, Pittsburgh, Pa.). Suitable optical dyes, including fluorophores, are described in Molecular Probes Handbook by Richard P. Haugland.

Suitable proteinaceous fluorescent labels also include, but are not limited to, green fluorescent protein, including a Renilla, Ptilosarcus, or Aequorea species of GFP (Chalfie et al., 1994, Science 263:802-805), EGFP (Clontech Laboratories, Inc., Genbank Accession Number U55762), blue fluorescent protein (BFP, Quantum Biotechnologies, Inc. 1801 de Maisonneuve Blvd. West, 8th Floor, Montreal, Quebec, Canada H3H 1J9; Stauber, 1998, Biotechniques 24:462-471; Heim et al., 1996, Curr. Biol. 6:178-182), enhanced yellow fluorescent protein (EYFP, Clontech Laboratories, Inc.), luciferase (Ichiki et al., 1993, J. Immunol. 150:5408-5417), β galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658, 5,418,155, 5,683,888, 5,741,668, 5,777,079, 5,804,387, 5,874,304, 5,876,995, 5,925,558).

Leucine zipper domains are peptides that promote oligomerization of the proteins in which they are found. Leucine zippers were originally identified in several DNA-binding proteins (Landschulz et al., 1988, Science 240:1759), and have since been found in a variety of different proteins. Among the known leucine zippers are naturally occurring peptides and derivatives thereof that dimerize or trimerize. Examples of leucine zipper domains suitable for producing soluble oligomeric proteins are described in PCT application WO 94/10308, and the leucine zipper derived from lung surfactant protein D (SPD) described in Hoppe et al., 1994, FEBS Letters 344:191. The use of a modified leucine zipper that allows for stable trimerization of a heterologous protein fused thereto is described in Fanslow et al., 1994, Semin. Immunol. 6:267-78. In one approach, recombinant fusion proteins comprising the target antigen antibody fragment or derivative fused to a leucine zipper peptide are expressed in suitable host cells, and the soluble oligomeric target antigen antibody fragments or derivatives that form are recovered from the culture supernatant.

The antibody construct of the invention may also comprise additional domains, which are e.g. helpful in the isolation of the molecule or relate to an adapted pharmacokinetic profile of the molecule. Domains helpful for the isolation of an antibody construct may be selected from peptide motives or secondarily introduced moieties, which can be captured in an isolation method, e.g. an isolation column. Non-limiting embodiments of such additional domains comprise peptide motives known as Myc-tag, HAT-tag, HA-tag, TAP-tag, GST-tag, chitin binding domain (CBD-tag), maltose binding protein (MBP-tag), Flag-tag, Strep-tag and variants thereof (e.g. StrepII-tag) and His-tag. All herein disclosed antibody constructs characterized by the identified CDRs are preferred to comprise a His-tag domain, which is generally known as a repeat of consecutive His residues in the amino acid sequence of a molecule, preferably of six His residues.

T cells or T lymphocytes are a type of lymphocyte (itself a type of white blood cell) that play a central role in cell-mediated immunity. There are several subsets of T cells, each with a distinct function. T cells can be distinguished from other lymphocytes, such as B cells and NK cells, by the presence of a T cell receptor (TCR) on the cell surface. The TCR is responsible for recognizing antigens bound to major histocompatibility complex (MHC) molecules and is composed of two different protein chains. In 95% of the T cells, the TCR consists of an alpha (a) and beta (β) chain. When the TCR engages with antigenic peptide and MHC (peptide/MHC complex), the T lymphocyte is activated through a series of biochemical events mediated by associated enzymes, co-receptors, specialized adaptor molecules, and activated or released transcription factors

The CD3 receptor complex is a protein complex and is composed of four chains. In mammals, the complex contains a CD3γ (gamma) chain, a CD3δ (delta) chain, and two CD3ε (epsilon) chains. These chains associate with the T cell receptor (TCR) and the so-called ζ (zeta) chain to form the T cell receptor CD3 complex and to generate an activation signal in T lymphocytes. The CD3γ (gamma), CD3δ (delta), and CD3ε (epsilon) chains are highly related cell-surface proteins of the immunoglobulin superfamily containing a single extracellular immunoglobulin domain. The intracellular tails of the CD3 molecules contain a single conserved motif known as an immunoreceptor tyrosine-based activation motif or ITAM for short, which is essential for the signaling capacity of the TCR. The CD3 epsilon molecule is a polypeptide which in humans is encoded by the CD3ε gene which resides on chromosome 11. The sequence of a preferred human CD3 epsilon extracellular domain is shown in SEQ ID NO: 1, and the most preferred CD3 binding epitope corresponding to amino acid residues 1-27 of the human CD3 epsilon extracellular domain is represented in SEQ ID NO: 2.

The redirected lysis of target cells via the recruitment of T cells by a multispecific, at least bispecific, antibody construct involves cytolytic synapse formation and delivery of perforin and granzymes. The engaged T cells are capable of serial target cell lysis, and are not affected by immune escape mechanisms interfering with peptide antigen processing and presentation, or clonal T cell differentiation; see, for example, WO 2007/042261.

Cytotoxicity mediated by bispecific antibody constructs can be measured in various ways. Effector cells can be e.g. stimulated enriched (human) CD8 positive T cells or unstimulated (human) peripheral blood mononuclear cells (PBMC). If the target cells are of macaque origin or express or are transfected with macaque target cell antigen, the effector cells should also be of macaque origin such as a macaque T cell line, e.g. 4119LnPx. The target cells should express (at least the extracellular domain of) target cell antigen, e.g. human or macaque target cell antigen. Target cells can be a cell line (such as CHO) which is stably or transiently transfected with target cell antigen, e.g. human or macaque target cell antigen. Alternatively, the target cells can be a target cell antigen positive natural expresser cell line, such as a human cancer cell line. Usually EC50 values are expected to be lower with target cell lines expressing higher levels of target cell antigen on the cell surface. The effector to target cell (E:T) ratio is usually about 10:1, but can also vary. Cytotoxic activity of bispecific antibody constructs can be measured in a ⁵¹chromium release assay (incubation time of about 18 hours) or in a in a FACS-based cytotoxicity assay (incubation time of about 48 hours). Modifications of the assay incubation time (cytotoxic reaction) are also possible. Other methods of measuring cytotoxicity are well-known to the skilled person and comprise MTT or MTS assays, ATP-based assays including bioluminescent assays, the sulforhodamine B (SRB) assay, WST assay, clonogenic assay and the ECIS technology.

The cytotoxic activity mediated by bispecific antibody constructs of the present invention is preferably measured in a cell-based cytotoxicity assay. It is represented by the EC₅₀ value, which corresponds to the half maximal effective concentration (concentration of the antibody construct which induces a cytotoxic response halfway between the baseline and maximum). Preferably, the EC₅₀ value of the bispecific antibody constructs is ≤20.000 pg/ml, more preferably ≤5000 pg/ml, even more preferably ≤1000 pg/ml, even more preferably ≤500 pg/ml, even more preferably ≤350 pg/ml, even more preferably ≤250 pg/ml, even more preferably ≤100 pg/ml, even more preferably ≤50 pg/ml, even more preferably ≤10 pg/ml, and most preferably ≤5 pg/ml.

Any of the above given EC₅₀ values can be combined with any one of the indicated scenarios of a cell-based cytotoxicity assay, e.g. in line with the methods described in the appended example. For example, when (human) CD8 positive T cells or a macaque T cell line are used as effector cells, the EC₅₀ value of the bispecific antibody construct of the invention (e.g. a target cell antigen/CD3 bispecific antibody construct) is preferably ≤1000 pg/ml, more preferably ≤500 pg/ml, even more preferably ≤250 pg/ml, even more preferably ≤100 pg/ml, even more preferably ≤50 pg/ml, even more preferably ≤10 pg/ml, and most preferably ≤5 pg/ml. If in this assay the target cells are (human or macaque) cells transfected with the target antigen (e.g. the target cell antigen CD33), such as CHO cells, the EC₅₀ value of the bispecific antibody construct is preferably ≤150 pg/ml, more preferably ≤100 pg/ml, even more preferably ≤50 pg/ml, even more preferably ≤30 pg/ml, even more preferably ≤10 pg/ml, and most preferably ≤5 pg/ml. If the target cells are a positive natural expresser cell line (e.g. of target cell antigen), then the EC₅₀ value is preferably ≤350 pg/ml, more preferably ≤250 pg/ml, even more preferably ≤200 pg/ml, even more preferably ≤100 pg/ml, even more preferably ≤150 pg/ml, even more preferably ≤100 pg/ml, and most preferably ≤50 pg/ml, or lower. When (human) PBMCs are used as effector cells, the EC₅₀ value of the bispecific antibody construct is preferably ≤1000 pg/ml, more preferably ≤750 pg/ml, more preferably ≤500 pg/ml, even more preferably ≤350 pg/ml, even more preferably ≤250 pg/ml, even more preferably ≤100 pg/ml, and most preferably ≤50 pg/ml, or lower.

Preferably, the bispecific antibody constructs of the present invention do not induce/mediate lysis or do not essentially induce/mediate lysis of target cell antigen negative cells such as CHO cells. The term “do not induce lysis”, “do not essentially induce lysis”, “do not mediate lysis” or “do not essentially mediate lysis” means that an antibody constructs of the present invention does not induce or mediate lysis of more than 30%, preferably not more than 20%, more preferably not more than 10%, particularly preferably not more than 9%, 8%, 7%, 6% or 5% of target cell antigen negative cells, whereby lysis of a target cell antigen positive cell line is set to be 100%. This usually applies for concentrations of the antibody construct of up to 500 nM. The skilled person knows how to measure cell lysis without further ado. Moreover, the present specification teaches specific instructions how to measure cell lysis.

Preferably, the bispecific antibody construct for the use according to the invention is administered according to a schedule comprising the following steps:
(a) administration of a first dose of the bispecific antibody construct, followed by
(b) administration of a second dose of the bispecific antibody construct, wherein said second dose exceeds said first dose, followed by
(c) administration of a third dose of the bispecific antibody construct, wherein said third dose exceeds said second dose, optionally followed by
(d) administration of a forth dose of the bispecific antibody construct, wherein said optional forth dose exceeds said third dose.

In line with the above it is further preferred that the period of administration of the first dose is up to seven days. This period of administration of the first dose may be used during the initial phase/first cycle of administration of the bispecific antibody construct e.g to reduce the tumor load in a patient (tumor debulking) while avoiding conditions such as cytokine storm and/or cytokine release syndrome which one might expect in case a higher dose is used during the period of administration of the first dose.

While in one embodiment of the invention the period of administration of the first dose is up to seven days, it is also within this preferred embodiment that this first dose is administered for a period of six days, five days, four days, three days, two days or one day. In the case that the tumor load or general condition of the individual patient does require the administration of the limited dose of the bispecific antibody construct in the first limited dose step, this first dose step is understood as a run-in phase/adaptation phase which should avoid or limit side effects resulting from the first contact of the patient with the bispecific antibody construct. A preferred range for a dose in such run-in phase/adaptation phase may be in a range of 1 to 50 μg/d, preferably in a range of 3 to 30 μg/d, further preferably in a range of 4 to 20 μg/d and even more preferably in a range of 5 to 15 μg/d for a canonical BITE® such as CD33×CD3 BISPECIFIC ANTIBODY CONSTRUCT, which is a 54 kDa single chain polypeptide. In a very preferred embodiment, the bispecific antibody construct according to the present invention is administered at a dose of 10 μg/d.

Preferred ranges for a second dose of the bispecific antibody construct are e.g. for a canonical BITE® such as CD33×CD3 BISPECIFIC ANTIBODY CONSTRUCT in the range of 10 μg/d to 10 mg/d, more preferably in the range of 25 μg/d to 1 mg/d and even more preferably in the range of 30 μg/d to 500 μg/d. In a very preferred embodiment, the second dose is 30 μg/d or 60 μg/d. In line with the above, the preferred ranges for the third dose of the bispecific antibody construct exceed the respective dose of the second dose. The third dose is typically in the range of 60 μg/d to 500 μg/d and preferably eradicates residual target cells which may have evaded treatment equivalent to the second dose according to the present invention.

It was surprisingly found that when a step dosing comprising at least two dosage steps is applied according to the present invention, then immunologic side effects such as undesired cytokine release, e.g. a cytokine release syndrome, may be effectively prevented. In contrast, if a dose equivalent to the second dosage is given without a prior lower dose equitant to the first dose of the present invention, then side effects, such as undesired cytokine release, e.g. a cytokine release syndrome, may occur. The same applies for the third dosage with respect to the second dosage.

It is also preferred for the present invention that the period of administration of the first and second dose is as short as possible to reach the target dose which addresses leukemic stem cells as soon as possible. This is decisive for therapeutic success with respect to an aggressive and progredient disease such as AML. Hence, it is a major achievement according to the present invention to provide a dosage scheme having a period of administration of the first dosage for only two or three days, preferably two days, and of two to four days for the second dosage. In turn the third dosage or the optional forth dosage, i.e. the target dosage, comprises a prolonged period of administration of preferably at least days.

Also in line with the present invention it is preferred for the bispecific antibody construct used in the treatment of myeloid leukemia that the first binding domain of the bispecific antibody construct comprises groups of six CDRs selected from the group consisting of SEQ ID NOs: 10 to 12 and 14 to 16, 22 to 24 and 26 to 28, 34 to 36 and 38 to 40, 46 to 48 and 50 to 52, 58 to 60 and 62 to 64, 70 to 72 and 74 to 76, 82 to 84 and 86 to 88, 94 to 96 an 98 to 100.

Moreover, in line with the present invention it is preferred for the bispecific antibody construct used in the treatment of myeloid leukemia that the second binding domain of the bispecific antibody construct comprises groups of six CDRs selected from the group consisting of SEQ ID NOs: 9 to 14, 27 to 32, 45 to 50, 63 to 68, 81 to 86, 99 to 104, 117 to 122, 135 to 140, 153 to 158 and 171 to 176 of WO 2008/119567.

As well as the second binding domain the first (or any further) binding domain(s) of the antibody construct of the invention is/are preferably cross-species specific for members of the mammalian order of primates. Cross-species specific CD3 binding domains are, for example, described in WO 2008/119567. According to one embodiment, the first and second binding domain, in addition to binding to human CD33 target cell antigen and human CD3, respectively, will also bind to the CD33 target cell antigen/CD3 of primates including (but not limited to) new world primates (such as Callithrix jacchus, Saguinus Oedipus or Saimiri sciureus), old world primates (such baboons and macaques), gibbons, and non-human homininae. Callithrix jacchus and Saguinus oedipus are both new world primate belonging to the family of Callitrichidae, while Saimiri sciureus is a new world primate belonging to the family of Cebidae.

In a preferred embodiment of the invention the bispecific antibody construct is a bispecific antibody construct. In line with the definitions provided herein above, this embodiment relates to bispecific antibody constructs, which are antibody constructs. In a preferred embodiment of the invention the bispecific antibody construct is a single chain construct. Such bispecific single chain antibody construct may comprise in line with the invention an amino acid sequence selected from the group consisting of SEQ ID NOs: 18, 19, 20, 30, 31, 32, 42, 43, 44, 54, 55, 56, 66, 67, 68, 78, 79, 80, 90, 91, 92, 102, 103, 104, 105, 106, 107 and 108.

Amino acid sequence modifications of the bispecific antibody constructs described herein are also contemplated. For example, it may be desirable to improve the binding affinity and/or other biological properties of the bispecific antibody construct. Amino acid sequence variants of the bispecific antibody constructs are prepared by introducing appropriate nucleotide changes into the bispecific antibody constructs nucleic acid, or by peptide synthesis. All of the below described amino acid sequence modifications should result in a bispecific antibody construct which still retains the desired biological activity (binding to target cell antigen and to CD3) of the unmodified parental molecule.

The term “amino acid” or “amino acid residue” typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (He or I): leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, He, Leu, Met, Phe, Pro, Val); a negatively charged side chain (e.g., Asp, Glu); a positively charged sidechain (e.g., Arg, His, Lys); or an uncharged polar side chain (e.g., Asn, Cys, Gln, Gly, His, Met, Phe, Ser, Thr, Trp, and Tyr).

Amino acid modifications include, for example, deletions from, and/or insertions into, and/or substitutions of, residues within the amino acid sequences of the bispecific antibody constructs. Any combination of deletion, insertion, and substitution is made to arrive at the final construct, provided that the final construct possesses the desired characteristics. The amino acid changes also may alter post-translational processes of the bispecific antibody constructs, such as changing the number or position of glycosylation sites.

For example, 1, 2, 3, 4, 5, or 6 amino acids may be inserted or deleted in each of the CDRs (of course, dependent on their length), while 1, 2, 3, 4, 5, 6, 7, 8, 9, 10, 11, 12, 13, 14, 15, 16, 17, 18, 19, 20, or 25 amino acids may be inserted or deleted in each of the FRs. Preferably, amino acid sequence insertions include amino- and/or carboxyl-terminal fusions ranging in length from 1, 2, 3, 4, 5, 6, 7, 8, 9 or 10 residues to polypeptides containing a hundred or more residues, as well as intra-sequence insertions of single or multiple amino acid residues. An insertional variant of the bispecific antibody construct of the invention includes the fusion to the N-terminus or to the C-terminus of the bispecific antibody construct to an enzyme or a fusion to a polypeptide which increases the serum half-life of the bispecific antibody construct.

An increased half-life is generally useful in in vivo applications of immunoglobulins, especially antibodies and most especially antibody fragments of small size. Although such antibody constructs based on antibody fragments (Fvs, disulphide bonded Fvs, Fabs, scFvs, dAbs) are able to rapidly reach most parts of the body, those antibody constructs are likely to suffer from rapid clearance from the body. Strategies described in the art for extending the half-life of antibody constructs such as single-chain diabodies include the conjugation of polyethylene glycol chains (PEGylation), the fusion to the IgG Fc region or to an albumin or albumin-binding domain.

Serum albumin is a protein physiologically produced by the liver; it occurs dissolved in blood plasma and is the most abundant blood protein in mammals. Albumin is essential for maintaining the oncotic pressure needed for proper distribution of body fluids between blood vessels and body tissues. It also acts as a plasma carrier by non-specifically binding several hydrophobic steroid hormones and as a transport protein for hemin and fatty acids. The term “serum albumin” respectively the human variant thereof (“human albumin”) defines in the context of the invented proteins either the parental human serum albumin protein (sequence as described in SEQ ID NO: 109) or any variant (e.g. such as albumin protein as depicted in SEQ ID NOs: 110-138) or fragment thereof preferably expressed as genetic fusion proteins and by chemical crosslinking etc. at least with one therapeutic protein. Variants comprising single or multiple mutations or fragments of albumin provide improved properties such as affinities to FcRn receptor and extended plasma half-life compared to its parent or reference. Variants of human albumin are described e.g. in WO 2014/072481. In line with the invention the serum albumin may be linked to the antibody construct via a peptide linker. It is preferred that the peptide linker has the amino acid sequence (GGGGS)ₙ (SEQ ID NO: 13)ₙ wherein “n” is an integer in the range of 1 to 5. Further preferred is that “n” is an integer in the range of 1 to 3, and most preferably “n” is 1 or 2.

The sites of greatest interest for substitutional mutagenesis include the CDRs of the heavy and/or light chain, in particular the hypervariable regions, but FR alterations in the heavy and/or light chain are also contemplated. The substitutions are preferably conservative substitutions as described herein. Preferably, 1, 2, 3, 4, 5, 6, 7, 8, 9, or 10 amino acids may be substituted in a CDR, while 1, 2, 3, 4, 5, 6, 7, 8, 9, 10, 11, 12, 13, 14, 15, 16, 17, 18, 19, 20, or 25 amino acids may be substituted in the framework regions (FRs), depending on the length of the CDR or FR. For example, if a CDR sequence encompasses 6 amino acids, it is envisaged that one, two or three of these amino acids are substituted. Similarly, if a CDR sequence encompasses 15 amino acids it is envisaged that one, two, three, four, five or six of these amino acids are substituted.

A useful method for identification of certain residues or regions of the bispecific antibody constructs that are preferred locations for mutagenesis is called “alanine scanning mutagenesis” as described by Cunningham and Wells in Science, 244: 1081-1085 (1989). Here, a residue or group of target residues within the bispecific antibody construct is/are identified (e.g. charged residues such as arg, asp, his, lys, and glu) and replaced by a neutral or negatively charged amino acid (most preferably alanine or polyalanine) to affect the interaction of the amino acids with the epitope.

Those amino acid locations demonstrating functional sensitivity to the substitutions then are refined by introducing further or other variants at, or for, the sites of substitution. Thus, while the site or region for introducing an amino acid sequence variation is predetermined, the nature of the mutation per se needs not to be predetermined. For example, to analyze or optimize the performance of a mutation at a given site, alanine scanning or random mutagenesis may be conducted at a target codon or region, and the expressed bispecific antibody construct variants are screened for the optimal combination of desired activity. Techniques for making substitution mutations at predetermined sites in the DNA having a known sequence are well known, for example, M13 primer mutagenesis and PCR mutagenesis. Screening of the mutants is done using assays of target antigen binding activities.

Generally, if amino acids are substituted in one or more or all of the CDRs of the heavy and/or light chain, it is preferred that the then-obtained “substituted” sequence is at least 60%, more preferably 65%, even more preferably 70%, particularly preferably 75%, more particularly preferably 80% identical to the “original” CDR sequence. This means that it is dependent of the length of the CDR to which degree it is identical to the “substituted” sequence. For example, a CDR having 5 amino acids is preferably 80% identical to its substituted sequence in order to have at least one amino acid substituted. Accordingly, the CDRs of the bispecific antibody construct may have different degrees of identity to their substituted sequences, e.g., CDRL1 may have 80%, while CDRL3 may have 90%.

Preferred substitutions (or replacements) are conservative substitutions. However, any substitution (including non-conservative substitution or one or more from the “exemplary substitutions” listed in Table 1, below) is envisaged as long as the bispecific antibody construct retains its capability to bind to target cell antigen via the first binding domain and to CD3 epsilon via the second binding domain and/or its CDRs have an identity to the then substituted sequence (at least 60%, more preferably 65%, even more preferably 70%, particularly preferably 75%, more particularly preferably 80% identical to the “original” CDR sequence).

Conservative substitutions are shown in Table 1 under the heading of “preferred substitutions”. If such substitutions result in a change in biological activity, then more substantial changes, denominated “exemplary substitutions” in Table 1, or as further described below in reference to amino acid classes, may be introduced and the products screened for a desired characteristic.

TABLE 1
Amino acid substitutions

| | Exemplary | Preferred |
|---|---|---|
| Original | Substitutions | Substitutions |
| Ala (A) | val, leu, ile | val |
| Arg (R) | lys, gln, asn | lys |
| Asn (N) | gln, his, asp, lys, arg | gln |
| Asp (D) | glu asn | glu |
| Cys (C) | ser, ala | ser |
| Gln (Q) | asn, glu | asn |
| Glu (E) | asp, gln | asp |
| Gly (G) | Ala | ala |
| His (H) | asn, gln, lys, arg | arg |
| Ile (I) | leu, val, met, ala, phe | leu |
| Leu (L) | norleucine, ile, val, met, ala | ile |
| Lys (K) | arg, gln, asn | arg |
| Met (M) | leu, phe, ile | leu |
| Phe (F) | leu, val, ile, ala, tyr | tyr |
| Pro (P) | Ala | ala |
| Ser (S) | Thr | thr |
| Thr (T) | Ser | ser |
| Trp (W) | tyr, phe | tyr |
| Tyr (Y) | trp, phe, thr, ser | phe |
| Val (V) | ile, leu, met, phe, ala | leu |

Substantial modifications in the biological properties of the bispecific antibody construct of the present invention are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties: (1) hydrophobic: norleucine, met, ala, val, leu, ile; (2) neutral hydrophilic: cys, ser, thr, asn, gln; (3) acidic: asp, glu; (4) basic: his, lys, arg; (5) residues that influence chain orientation: gly, pro; and (6) aromatic: trp, tyr, phe.

Non-conservative substitutions will entail exchanging a member of one of these classes for another class. Any cysteine residue not involved in maintaining the proper conformation of the bispecific antibody construct may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

For amino acid sequences, sequence identity and/or similarity is determined by using standard techniques known in the art, including, but not limited to, the local sequence identity algorithm of Smith and Waterman, 1981, Adv. Appl. Math. 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., 1984, Nucl. Acid Res. 12:387-395, preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, 1987, J. Mol. Evol. 35:351-360; the method is similar to that described by Higgins and Sharp, 1989, CABIOS 5:151-153. Useful PILEUP parameters including a default gap weight of 3.00, a default gap length weight of 0.10, and weighted end gaps.

Another example of a useful algorithm is the BLAST algorithm, described in: Altschul et al., 1990, J. Mol. Biol. 215:403-410; Altschul et al., 1997, Nucleic Acids Res. 25:3389-3402; and Karin et al., 1993, Proc. Natl. Acad. Sci. U.S.A. 90:5873-5787. A particularly useful BLAST program is the WU-BLAST-2 program which was obtained from Altschul et al., 1996, Methods in Enzymology 266:460-480. WU-BLAST-2 uses several search parameters, most of which are set to the default values. The adjustable parameters are set with the following values: overlap span=1, overlap fraction=0.125, word threshold (T)=II. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., 1993, Nucl. Acids Res. 25:3389-3402. Gapped BLAST uses BLOSUM-62 substitution scores; threshold T parameter set to 9; the two-hit method to trigger ungapped extensions, charges gap lengths of k a cost of 10+k; Xu set to 16, and Xg set to 40 for database search stage and to 67 for the output stage of the algorithms. Gapped alignments are triggered by a score corresponding to about 22 bits.

Generally, the amino acid homology, similarity, or identity between individual variant CDRs are at least 60% to the sequences depicted herein, and more typically with preferably increasing homologies or identities of at least 65% or 70%, more preferably at least 75% or 80%, even more preferably at least 85%, 90%, 91%, 92%, 93%, 94%, 95%, 96%, 97%, 98%, 99%, and almost 100%. In a similar manner, “percent (%) nucleic acid sequence identity” with respect to the nucleic acid sequence of the binding proteins identified herein is defined as the percentage of nucleotide residues in a candidate sequence that are identical with the nucleotide residues in the coding sequence of the bispecific antibody construct. A specific method utilizes the BLASTN module of WU-BLAST-2 set to the default parameters, with overlap span and overlap fraction set to 1 and 0.125, respectively.

Generally, the nucleic acid sequence homology, similarity, or identity between the nucleotide sequences encoding individual variant CDRs and the nucleotide sequences depicted herein are at least 60%, and more typically with preferably increasing homologies or identities of at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%, and almost 100%. Thus, a “variant CDR” is one with the specified homology, similarity, or identity to the parent CDR of the invention, and shares biological function, including, but not limited to, at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR.

In one embodiment the bispecific antibody construct for the use in accordance with this invention is administered in combination with one or more epigenetic factors selected from the group consisting of histone deacetylase (HDAC) inhibitors, DNA methyltransferase (DNMT) I inhibitors, hydroxyurea, Granulocyte-Colony Stimulating Factor (G-CSF), histone demethylase inhibitors and ATRA (All Trans-retinoic acid) and wherein:
(a) the one or more epigenetic factors are administered prior to the administration of the bispecific antibody construct;
(b) the one or more epigenetic factors are administered subsequent to the administration of the bispecific antibody construct; or
(c) the one or more epigenetic factors and the bispecific antibody construct are administered simultaneously.

The term “epigenetic factor” in connection with the present invention defines a compound which is capable of changing the gene expression or cellular phenotype of a cell population upon administration. It is understood that such change refers to one or more functional relevant modifications to the genome without involving a change in the nucleic acid sequence. Examples of such modifications are DNA methylation and histone modification, which are both important for the regulation of gene expression without altering the underlying DNA sequence.

Details for a treatment of myeloid leukemia comprising the administration of the bispecific antibody construct in combination with one or more of the above described epigenetic factors have been provided in PCT/EP2014/069575.

In one embodiment of the invention it is preferred that the one or more epigenetic factors are administered up to seven days prior to the administration of the bispecific antibody construct. Also in one embodiment of the invention it is preferred that the epigenetic factor is hydroxyurea.

It is preferred for the present invention that the myeloid leukemia is selected from the group consisting of acute myeloblastic leukemia, chronic neutrophilic leukemia, myeloid dendritic cell leukemia, accelerated phase chronic myelogenous leukemia, acute myelomonocytic leukemia, juvenile myelomonocytic leukemia, chronic myelomonocytic leukemia, acute basophilic leukemia, acute eosinophilic leukemia, chronic eosinophilic leukemia, acute megakaryoblastic leukemia, essential thrombocytosis, acute erythroid leukemia, polycythemia vera, myelodysplastic syndrome, acute panmyeloic leukemia, myeloid sarcoma, and acute biphenotypic leukaemia. More preferably, the myeloid leukemia is an acute myeloid leukemia (AML). The definition of AML inter alia comprises acute myeloblastic leukemia, acute myeloid dendritic cell leukemia, acute myelomonocytic leukemia, acute basophilic leukemia, acute eosinophilic leukemia, acute megakaryoblastic leukemia, acute erythroid leukemia, and acute panmyeloic leukemia

The bispecific antibody construct described in connection with this invention may be formulated for an appropriate administration to a subject in the need thereof in form of a pharmaceutical composition.

Formulations described herein are useful as pharmaceutical compositions in the treatment, amelioration and/or prevention of the pathological medical condition as described herein in a patient in need thereof. The term “treatment” refers to both therapeutic treatment and prophylactic or preventative measures. Treatment includes the application or administration of the formulation to the body, an isolated tissue, or cell from a patient who has a disease/disorder, a symptom of a disease/disorder, or a predisposition toward a disease/disorder, with the purpose to cure, heal, alleviate, relieve, alter, remedy, ameliorate, improve, or affect the disease, the symptom of the disease, or the predisposition toward the disease.

The term “disease” refers to any condition that would benefit from treatment with the bispecific antibody construct or the pharmaceutical composition described herein. This includes chronic and acute disorders or diseases including those pathological conditions that predispose the mammal to the disease in question.

The terms “subject in need” or those “in need of treatment” includes those already with the disorder, as well as those in which the disorder is to be prevented. The subject in need or “patient” includes human and other mammalian subjects that receive either prophylactic or therapeutic treatment.

The bispecific antibody construct of the invention will generally be designed for specific routes and methods of administration, for specific dosages and frequencies of administration, for specific treatments of specific diseases, with ranges of bio-availability and persistence, among other things. The materials of the composition are preferably formulated in concentrations that are acceptable for the site of administration.

Formulations and compositions thus may be designed in accordance with the invention for delivery by any suitable route of administration. In the context of the present invention, the routes of administration include, but are not limited to
topical routes (such as epicutaneous, inhalational, nasal, opthalmic, auricular/aural, vaginal, mucosal);
enteral routes (such as oral, gastrointestinal, sublingual, sublabial, buccal, rectal); and
parenteral routes (such as intravenous, intraarterial, intraosseous, intramuscular, intracerebral, intracerebroventricular, epidural, intrathecal, subcutaneous, intraperitoneal, extra-amniotic, intraarticular, intracardiac, intradermal, intralesional, intrauterine, intravesical, intravitreal, transdermal, intranasal, transmucosal, intrasynovial, intraluminal).

The pharmaceutical compositions and the bispecific antibody construct described in connection with the invention are particularly useful for parenteral administration, e.g., subcutaneous or intravenous delivery, for example by injection such as bolus injection, or by infusion such as continuous infusion. Pharmaceutical compositions may be administered using a medical device. Examples of medical devices for administering pharmaceutical compositions are described in U.S. Pat. Nos. 4,475,196; 4,439,196; 4,447,224; 4,447, 233; 4,486,194; 4,487,603; 4,596,556; 4,790,824; 4,941,880; 5,064,413; 5,312,335; 5,312,335; 5,383,851; and 5,399,163.

In particular, the present invention provides for an uninterrupted administration of the suitable composition. As a non-limiting example, uninterrupted or substantially uninterrupted, i.e. continuous administration may be realized by a small pump system worn by the patient for metering the influx of therapeutic agent into the body of the patient. The pharmaceutical composition comprising the bispecific antibody construct described in connection with the invention can be administered by using said pump systems. Such pump systems are generally known in the art, and commonly rely on periodic exchange of cartridges containing the therapeutic agent to be infused. When exchanging the cartridge in such a pump system, a temporary interruption of the otherwise uninterrupted flow of therapeutic agent into the body of the patient may ensue. In such a case, the phase of administration prior to cartridge replacement and the phase of administration following cartridge replacement would still be considered within the meaning of the pharmaceutical means and methods of the invention together make up one “uninterrupted administration” of such therapeutic agent.

The continuous or uninterrupted administration of the bispecific antibody construct described in connection with the invention may be intravenous or subcutaneous by way of a fluid delivery device or small pump system including a fluid driving mechanism for driving fluid out of a reservoir and an actuating mechanism for actuating the driving mechanism. Pump systems for subcutaneous administration may include a needle or a cannula for penetrating the skin of a patient and delivering the suitable composition into the patient's body. Said pump systems may be directly fixed or attached to the skin of the patient independently of a vein, artery or blood vessel, thereby allowing a direct contact between the pump system and the skin of the patient. The pump system can be attached to the skin of the patient for 24 hours up to several days. The pump system may be of small size with a reservoir for small volumes. As a non-limiting example, the volume of the reservoir for the suitable pharmaceutical composition to be administered can be between 0.1 and 50 ml.

The continuous administration may also be transdermal by way of a patch worn on the skin and replaced at intervals. One of skill in the art is aware of patch systems for drug delivery suitable for this purpose. It is of note that transdermal administration is especially amenable to uninterrupted administration, as exchange of a first exhausted patch can advantageously be accomplished simultaneously with the placement of a new, second patch, for example on the surface of the skin immediately adjacent to the first exhausted patch and immediately prior to removal of the first exhausted patch. Issues of flow interruption or power cell failure do not arise.

If the pharmaceutical composition has been lyophilized, the lyophilized material is first reconstituted in an appropriate liquid prior to administration. The lyophilized material may be reconstituted in, e.g., bacteriostatic water for injection (BWFI), physiological saline, phosphate buffered saline (PBS), or the same formulation the protein had been in prior to lyophilization.

The compositions of the present invention can be administered to the subject at a suitable dose which can be determined e.g. by dose escalating studies by administration of increasing doses of the bispecific antibody construct described in connection with the invention exhibiting cross-species specificity described herein to non-chimpanzee primates, for instance macaques. As set forth above, the bispecific antibody construct described in connection with the invention exhibiting cross-species specificity described herein can be advantageously used in identical form in preclinical testing in non-chimpanzee primates and as drug in humans. The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts or doses effective for this use will depend on the condition to be treated (the indication), the delivered bispecific antibody construct, the therapeutic context and objectives, the severity of the disease, prior therapy, the patient's clinical history and response to the therapeutic agent, the route of administration, the size (body weight, body surface or organ size) and/or condition (the age and general health) of the patient, and the general state of the patient's own immune system. The proper dose can be adjusted according to the judgment of the attending physician such that it can be administered to the patient once or over a series of administrations, and in order to obtain the optimal therapeutic effect.

A therapeutic effective amount of a bispecific antibody construct described in connection with the invention preferably results in a decrease in severity of disease symptoms, an increase in frequency or duration of disease symptom-free periods or a prevention of impairment or disability due to the disease affliction. For treating target cell antigen-expressing tumors, a therapeutically effective amount of the bispecific antibody construct described in connection with the invention, e.g. an anti-target cell antigen/anti-CD3 antibody construct, preferably inhibits cell growth or tumor growth by at least about 20%, at least about 40%, at least about 50%, at least about 60%, at least about 70%, at least about 80%, or at least about 90% relative to untreated patients. The ability of a compound to inhibit tumor growth may be evaluated in an animal model predictive of efficacy in human tumors.

The pharmaceutical composition can be administered as a sole therapeutic or in combination with additional therapies such as anti-cancer therapies as needed, e.g. other proteinaceous and non-proteinaceous drugs. These drugs may be administered simultaneously with the composition comprising the bispecific antibody construct described in connection with the invention as defined herein or separately before or after administration of said bispecific antibody construct in timely defined intervals and doses.

Further, the present inventors observed that rare side effects, such as immunologic side effects could be prevented or alleviated by means of a glucocorticoid (pre) and/or (co) therapy.

Accordingly, the present invention establishes that glucocorticoids such as dexamethasone mitigate or even prevent adverse effects which might occur in the course of a treatment with CD33/CD3 specific bispecific antibody constructs according to the present invention.

Glucocorticoids (GCs) are still the most widely used immunosuppressive agents for the treatment of inflammatory disorders and autoimmune diseases. Glucocorticoids (GC) are a class of steroid hormones that bind to the glucocorticoid receptor (GR), which is present in almost every vertebrate animal cell, including humans. These compounds are potent anti-inflammatory agents, regardless of the inflammation's cause. Glucocorticoids suppress, inter alia, the cell-mediated immunity by inhibiting genes that code for the cytokines IL-1, IL-2, IL-3, IL-4, IL-5, IL-6, IL-8 and IFN-γ.

Cortisone which belongs to the group of GCs is an important therapeutic drug which is used to fight many ailments ranging from Addison's disease to rheumatoid arthritis. Ever since the discovery of its anti-rheumatic properties, which led to its acclaim as a wonder drug, many derivatives of cortisone with enhanced properties to better fight a specific ailment have been produced. Cortisone belongs to a group of steroids known as corticosteroids. These steroids are produced by the adrenal cortex, which is the outer part of the adrenal glands, near the kidneys. The corticosteroids are divided into two main groups: the glucocorticoids (GCs), which control fat, protein, calcium and carbohydrate metabolism, and the mineralocorticoids controlling sodium and potassium levels. Cortisone belongs to the former group, i.e. to the GCs. Cortisone and its many derivatives are used for a variety of diseases. Cortisone also helped to make organ transplants a reality due to its ability to minimize the defence reaction of the body towards foreign proteins present in the implanted organ and thus damage the functionality of the implanted organ. However, despite clinical use during more than 50 years, the specific anti-inflammatory effects of GC on different cellular compartments of the immune system are not yet clear. GCs affect nearly every cell of the immune system, and there is growing evidence for cell type-specific mechanisms.

In one specific embodiment, the present invention relates to a glucocorticoid (GC) for use in the amelioration, treatment or prophylaxis of adverse effects caused by a CD33/CD3 bispecific antibody construct. As outlined above, these unwanted adverse effects may be prevented by a step dosing as described herein. However, for mere precaution, glucocorticoid(s) for use in the amelioration, treatment or prophylaxis of (immunological) adverse effects in a patient may be provided wherein said patient is subject to therapy with a CD33/CD3 bispecific antibody construct. Accordingly, in one further aspect the present invention relates to a glucocorticoid (GC) for use in a method in the amelioration, treatment or prophylaxis of immunological adverse effects caused by a CD33/CD3 bispecific antibody construct according to the present invention.

Also, the present invention relates to a method of amelioration, treatment or prophylaxis of immunological adverse effects caused by a CD33/CD3 bispecific antibody construct, said method comprising administering to a patient in need thereof IL-6R blocking antibody tocilizumab or a glucocorticoid (GC). The GC is preferably administered in an amount which is sufficient to ameliorate, treat or prevent said immunological adverse effects caused by a CD33/CD3 bispecific antibody construct.

The term “glucocorticoid” means compounds that bind, preferably specifically, to the glucocorticoid receptor. Said term includes compound(s) selected from the group consisting of cortisone, cortisol (hydrocortisone), cloprednol, prednisone, prednisolone, methylprednisolone, deflazacort, fluocortolone, triamcinolone, dexamethasone, betamethasone, cortivazol, paramethasone, and/or fluticasone, including pharmaceutically acceptable derivatives thereof. In the context f the embodiments of the present invention, the mentioned compounds may be used alone or in combination. Dexamethasone is preferred. The present invention is however not limited to the above mentioned specific GCs. It is envisaged that all substances which already are or will be classified as a “glucocorticoid”, may be employed in the context of the present invention as well. Such future glucocorticoids include compounds which specifically bind to and activate the glucocorticoid receptor. The term “specifically binds to the GC receptor” means in accordance with the present invention that the GC (or a compound which is assumed to act like a GC) associates with (e.g., interacts with) the GC receptor (also known as NR3C1) to a statistically significant degree as compared to association with proteins/receptors generally (i.e., non-specific binding). When the GC receptor binds to glucocorticoids, its primary mechanism of action is the regulation of gene transcription. In the absence of GC, the glucocorticoid receptor (GR) resides in the cytosol complexed with a variety of proteins including heat shock protein 90 (hsp90), the heat shock protein 70 (hsp70) and the protein FKBP52 (FK506-binding protein 52). The binding of the GC to the glucocorticoid receptor (GR) results in release of the heat shock proteins. It is thus envisaged that a future GC, or a pharmaceutically acceptable derivative or salt of a GC is preferably able to bind to the GC receptor and to release the above mentioned heat shock proteins. The activated GR complex up-regulates the expression of anti-inflammatory proteins in the nucleus or represses the expression of pro-inflammatory proteins in the cytosol (by preventing the translocation of other transcription factors from the cytosol into the nucleus).

In a preferred embodiment, said GC is selected from the most clinical used and relevant GCs like dexamethasone, fluticasonepropionate, prednisolone, methylprednisolone, betamethasone, triamcinolonacetonide or combinations thereof.

In an even more preferred embodiment, said GC is dexamethasone.

Dexamethasone has the highest glucocorticoid potency of the most commonly used steroids and also has the longest half-life (see Table 2 below). But a person skilled in the field can select one of the other known glucocorticoids, some of which are disclosed herein, and select an appropriate effective dose to ameliorate or prevent immunological adverse events that may result from the treatment of a patient in need thereof.

TABLE 2
steroid dosing

| | | Relative | | |
|---|---|---|---|---|
| | Approx. | anti- | Relative | |
| | equiv. | inflammatory | mineralocorticoid | Biologic |
| | dose | (glucocorticoid) | (Na⁺retaning) | half-life |
| Agent | (mg) | potency | potency | (hrs) |
| Cortisone | 25 | 0.8 | 0.8 | 8-12 |
| Hydrocortisone | 20 | 1 | 1 | 8-12 |
| Prednisone | 5 | 4 | 0.8 | 18-36 |
| Prednisolone | 5 | 4 | 0.8 | 18-36 |
| Methyl- | 5 | 5 | 0.5 | 18-36 |
| prednisolone | | | | |
| Dexamethasone | 0.75 | 25 | 0 | 36-54 |

Dexamethasone also possesses a beneficial effect in malignant central nervous system (CNS) disease (e.g. CNS lymphoma or brain metastases)—possibly due to specific penetration to the CNS. It is also preferentially (over other steroids) used to treat brain edema. Although corticosteroids decrease capillary permeability in the tumor itself, it has been found in animal models that dexamethasone may act differently and decrease edema by effects on bulk flow away from the tumor (Molnar, Lapin, & Goothuis, 1995, Neurooncol. 1995; 25(1):19-28.

For the clinical trials in connection with the application of a CD33/CD3 bispecific antibody construct, the present inventors had to develop a treatment regime which was efficient and would be well tolerated by most of the patients. To this end, the present inventors applied a step-wise application of a CD33/CD3 bispecific antibody construct as outlined herein. Thereby, adverse effects could be reduced in number, ameliorated and even prevented.

The dose of the GC that is to be used in accordance with the embodiments of the present invention is not limited, i.e. it will depend on the circumstances of the individual patient. GC can be administered intravenously or orally. Preferred dosages of the GC include, however, between 1 to 6 mg (dexamethasone equivalent) at the lower end of dosing to 40 mg (dexamethasone equivalent). Said dosage can be administered all at once or subdivided into smaller dosages. Preferred is a subdivide dose wherein one dose of GC is given prior to the infusion of the first and/or second dose according to the step dosing as described herein, and the other dose of GC is given prior to the administration of the second or third dose according to the step dosing as described herein. Hence, GC is preferably two times dosed per treatment cycle. Even more preferably, GC is administered one 24 or 8 h or 4 h or 1 h before the beginning of a treatment cycle or the beginning of the administration of the next higher dose within said treatment cycle. In this regard, 1 h is most preferred. The dose is 1 to 40 mg each, preferably 5 to 20 mg, most preferably 8 mg each. “d” denotes one day. Further dosage regimens are derivable from the appended examples. All dosages given in this paragraph refer to dexamethasone equivalents.

The term “effective and non-toxic dose” as used herein refers to a tolerable dose of a bispecific antibody construct which is high enough to cause depletion of pathologic cells, tumor elimination, tumor shrinkage or stabilization of disease without or essentially without major toxic effects. Such effective and non-toxic doses may be determined e.g. by dose escalation studies described in the art and should be below the dose inducing severe adverse side events (dose limiting toxicity, DLT).

Alternatively, tocilizumab may be used in premedication.

The term “toxicity” as used herein refers to the toxic effects of a drug manifested in adverse events or severe adverse events. These side events might refer to a lack of tolerability of the drug in general and/or a lack of local tolerance after administration. Toxicity could also include teratogenic or carcinogenic effects caused by the drug.

The term “safety”, “in vivo safety” or “tolerability” as used herein defines the administration of a drug without inducing severe adverse events directly after administration (local tolerance) and during a longer period of application of the drug. “Safety”, “in vivo safety” or “tolerability” can be evaluated e.g. at regular intervals during the treatment and follow-up period. Measurements include clinical evaluation, e.g. organ manifestations, and screening of laboratory abnormalities. Clinical evaluation may be carried out and deviations to normal findings recorded/coded according to NCI-CTC and/or MedDRA standards. Organ manifestations may include criteria such as allergy/immunology, blood/bone marrow, cardiac arrhythmia, coagulation and the like, as set forth e.g. in the Common Terminology Criteria for adverse events v4 (CTCAE). Laboratory parameters which may be tested include for instance hematology, clinical chemistry, coagulation profile and urine analysis and examination of other body fluids such as serum, plasma, lymphoid or spinal fluid, liquor and the like. Safety can thus be assessed e.g. by physical examination, imaging techniques (i.e. ultrasound, x-ray, CT scans, Magnetic Resonance Imaging (MRI), other measures with technical devices (i.e. electrocardiogram), vital signs, by measuring laboratory parameters and recording adverse events. For example, adverse events in non-chimpanzee primates in the uses and methods according to the invention may be examined by histopathological and/or histochemical methods.

The above terms are also referred to e.g. in the Preclinical safety evaluation of biotechnology-derived pharmaceuticals S6; ICH Harmonised Tripartite Guideline; ICH Steering Committee meeting on Jul. 16, 1997.

In a preferred embodiment of the method of the invention only the first cycle of the treatment comprises the administration according to step (a), whereas the following cycles start with the dose according to step (b), (c) or (d).

It is preferred for the method of the invention that the first binding domain of the bispecific antibody construct comprises groups of six CDRs selected from the group consisting of SEQ ID NOs: 10 to 12 and 14 to 16, 22 to 24 and 26 to 28, 34 to 36 and 38 to 40, 46 to 48 and 50 to 52, 58 to 60 and 62 to 64, 70 to 72 and 74 to 76, 82 to 84 and 86 to 88, 94 to 96 an 98 to 100.

Also in line with a preferred embodiment of the method of the invention the second binding domain of the bispecific antibody construct comprises groups of six CDRs selected from the group consisting of SEQ ID NOs: 9 to 14, 27 to 32, 45 to 50, 63 to 68, 81 to 86, 99 to 104, 117 to 122, 135 to 140, 153 to 158 and 171 to 176 of WO 2008/119567.

In a preferred embodiment of the method of the invention the bispecific antibody construct is a bispecific antibody construct.

Moreover, it is preferred for the method of the invention that the bispecific antibody construct is a single chain construct comprising an amino acid sequence selected from the group consisting of SEQ ID NOs: 18, 19, 20, 30, 31, 32, 42, 43, 44, 54, 55, 56, 66, 67, 68, 78, 79, 80, 90, 91, 92, 102, 103, 104, 105, 106, 107 and 108.

In one embodiment of the method of the invention the bispecific antibody construct is administered in combination with one or more epigenetic factors selected from the group consisting of histone deacetylase (HDAC) inhibitors, DNA methyltransferase (DNMT) I inhibitors, hydroxyurea, Granulocyte-Colony Stimulating Factor (G-CSF), histone demethylase inhibitors and ATRA (All Trans-retinoic acid) and wherein:
(a) the one or more epigenetic factors are administered prior to the administration of the bispecific antibody construct;
(b) the one or more epigenetic factors are administered subsequent to the administration of the bispecific antibody construct; or
(c) the one or more epigenetic factors and the bispecific antibody construct are administered simultaneously.

It is preferred for the method of the invention that the one or more epigenetic factors are administered up to seven days prior to the administration of the bispecific antibody construct. For one embodiment of the method of the invention it is preferred that the epigenetic factor is hydroxyurea

As described herein above, in line with the present invention the myeloid leukemia is selected from the group consisting of acute myeloblastic leukemia, chronic neutrophilic leukemia, myeloid dendritic cell leukemia, accelerated phase chronic myelogenous leukemia, acute myelomonocytic leukemia, juvenile myelomonocytic leukemia, chronic myelomonocytic leukemia, acute basophilic leukemia, acute eosinophilic leukemia, chronic eosinophilic leukemia, acute megakaryoblastic leukemia, essential thrombocytosis, acute erythroid leukemia, polycythemia vera, myelodysplastic syndrome, acute panmyeloic leukemia, myeloid sarcoma, and acute biphenotypic leukaemia. It is preferred that the myeloid leukemia is an acute myeloid leukemia (AML).

Also in one embodiment the invention provides a use of a bispecific antibody construct comprising a first binding domain specifically binding to CD33 and a second binding domain specifically binding to CD3 preferably for the preparation of a pharmaceutical composition for the treatment of myeloid leukemia, wherein the bispecific antibody construct is to be administered for more than 14 days followed by a period of at least 14 days without administration of the construct.

It is preferred of the use of the invention that the bispecific antibody construct is to be administered according to a schedule comprising the following steps:
(a) administration of a first dose of the bispecific antibody construct, followed by
(b) administration of a second dose of the bispecific antibody construct, wherein said second dose exceeds said first dose, followed by
(c) administration of a third dose of the bispecific antibody construct, wherein said optional third dose exceeds said second dose, optionally followed by
(d) administration of a forth dose of the bispecific antibody construct, wherein said optional third dose exceeds said third dose.

In a preferred embodiment of the use of the invention only the first cycle of the treatment comprises the administration according to step (a), whereas the following cycles start with the dose according to step (b), (c) or (d).

It is preferred for the use of the invention that the first binding domain of the bispecific antibody construct comprises groups of six CDRs selected from the group consisting of SEQ ID NOs: 10 to 12 and 14 to 16, 22 to 24 and 26 to 28, 34 to 36 and 38 to 40, 46 to 48 and 50 to 52, 58 to 60 and 62 to 64, 70 to 72 and 74 to 76, 82 to 84 and 86 to 88, 94 to 96 an 98 to 100.

Also in line with a preferred embodiment of the use of the invention the second binding domain of the bispecific antibody construct comprises groups of six CDRs selected from the group consisting of SEQ ID NOs: 9 to 14, 27 to 32, 45 to 50, 63 to 68, 81 to 86, 99 to 104, 117 to 122, 135 to 140, 153 to 158 and 171 to 176 of WO 2008/119567.

In a preferred embodiment of the use of the invention the bispecific antibody construct is a bispecific antibody construct.

Moreover, it is preferred for the use of the invention that the bispecific antibody construct is a single chain construct comprising an amino acid sequence selected from the group consisting of SEQ ID NOs: 18, 19, 20, 30, 31, 32, 42, 43, 44, 54, 55, 56, 66, 67, 68, 78, 79, 80, 90, 91, 92, 102, 103, 104, 105, 106, 107 and 108.

In one embodiment of the use of the invention the bispecific antibody construct is administered in combination with one or more epigenetic factors selected from the group consisting of histone deacetylase (HDAC) inhibitors, DNA methyltransferase (DNMT) I inhibitors, hydroxyurea, Granulocyte-Colony Stimulating Factor (G-CSF), histone demethylase inhibitors and ATRA (All Trans-retinoic acid) and wherein:
(a) the one or more epigenetic factors are administered prior to the administration of the bispecific antibody construct;
(b) the one or more epigenetic factors are administered subsequent to the administration of the bispecific antibody construct; or
(c) the one or more epigenetic factors and the bispecific antibody construct are administered simultaneously.

It is preferred for the use of the invention that the one or more epigenetic factors are administered up to seven days prior to the administration of the bispecific antibody construct. For one embodiment of the use of the invention it is preferred that the epigenetic factor is hydroxyurea

As described herein above, in line with the present invention the myeloid leukemia is selected from the group consisting of acute myeloblastic leukemia, chronic neutrophilic leukemia, myeloid dendritic cell leukemia, accelerated phase chronic myelogenous leukemia, acute myelomonocytic leukemia, juvenile myelomonocytic leukemia, chronic myelomonocytic leukemia, acute basophilic leukemia, acute eosinophilic leukemia, chronic eosinophilic leukemia, acute megakaryoblastic leukemia, essential thrombocytosis, acute erythroid leukemia, polycythemia vera, myelodysplastic syndrome, acute panmyeloic leukemia, myeloid sarcoma, and acute biphenotypic leukaemia. It is preferred that the myeloid leukemia is an acute myeloid leukemia (AML).

The patient population considered susceptible for the present inventive method is AML as defined by the WHO Classification persisting or recurring following one or more treatment courses except promyelocytic leukemia (APML). The patient population may comprise AML secondary to prior myelodysplastic syndrome. Preferably, the patient population comprises AML as defined by the WHO Classification either persisting/refractory after at least 1 primary induction courses (i.e, no response after at least 1 prior chemotherapy cycles) or recurring after having achieved an initial response to chemotherapy except promyelocytic leukemia (APML) and except AML secondary to prior myelodysplastic syndrome. Further, the preferred patient population is characterized by having more than 1% blasts in bone marrow, preferably more than 5% blasts. Typically, patient population ECOG performance status is less than 2.

### General Definitions

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within ±20%, preferably within ±15%, more preferably within ±10%, and most preferably within ±5% of a given value or range.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that the inventions herein are not limited to particular methodology, protocols, or reagents, as such can vary. The discussion and examples provided herein are presented for the purpose of describing particular embodiments only and are not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

### EXAMPLES

The following examples are provided for the purpose of illustrating specific embodiments or features of the present invention. These examples should not be construed as to limit the scope of this invention. The examples are included for purposes of illustration, and the present invention is limited only by the claims.

### Example 1

The objective of this study was to evaluate outcomes of patients presenting with r/r AML.

### Methods

Data are derived from the MD Anderson Cancer Center Leukemia Central Data Repository, a comprehensive collection of clinical data that reflects the experiences of patients with leukemia. Included patients were treated for r/r AML for at least one treatment course at MDACC between the years 2002 and 2016. At the time of inclusion in this study, patients had at least one prior treatment failure, years old at time of AML diagnosis, and no testes or CNS extramedullary disease. Acute promyelocytic leukemia diagnoses were excluded.

Descriptive characteristics of patients were summarized with a mean and standard deviation and/or median and interquartile range. Complete remission (CR) and complete remission with incomplete hematologic recovery (CRi) rates were described as proportion with Wald 95% confidence intervals. Time to event analyses estimated using the Kaplan-Meier median and probabilities at specific time intervals 3/6/9/12 months with Wald 95% confidence intervals. Subgroup analyses used Wald chi-square tests.

### Results

A total of 1021 patients were included. Median age of included patients were 60 years old, first relapsed/refractory AML occurred in year 2011-2016 for 43% (n=439) of patients. At least one cytogenetic abnormality was present in 53.3% (n=546) of the population, 34.5% (n=352) had a history of an antecedent hematologic disease, and 10.5% (n=107) were therapy induced AML. For patients with available induction records, approximately 46% (295/635) were refractory. Among patients that achieved a CR to induction, 45% (118/264) had <6 months duration of CR. Overall, only a small proportion of all r/r AML patients are able to achieve a second complete remission (CR2). CR rates decreases with each subsequent salvage attempt (Table 1). Rates are lower amongst patients >60 years of age. Among various types of salvage regimens, the range of CR was from 0 to 36%. Regimens based on high dose cytarabine were the most common (n=299). Although sample size was modest, regimens containing a FLT3 inhibitor induced the highest CR and CR/CRi rates (36% CR2, 33% CR3) (Table 2). Age, cytogenetics, antecedent disease, duration of first remission, and year of relapse were associated with CR rates.

Overall survival and event free survival were modest and decreased with subsequent salvage (Table 3). Age, cytogenetics, antecedent disease, De novo/therapy-induced AML, Duration of first remission, and platelet count were associated with survival.

### Conclusion

Overall, most patients were unable to achieve a second or even more CR2. Fewer patients are able to achieve subsequent CR after a second treatment failure or relapse. EFS is short due to most patients failing to achieve CR. Even at first salvage, OS is less than 1 year. These data demonstrate that relapsed or refractory AML patients have poor overall outcomes and the need for additional options for patients. These data can be used to help guide the development of novel protocols and therapeutic options in AML for a variety of different endpoints.

### Example 2

The objectives of this study was to evaluate the safety, pharmacokinetics, and pharmacodynamics of CD33×CD3 BISPECIFIC ANTIBODY CONSTRUCT in R/R AML and to estimate the maximum tolerated dose.

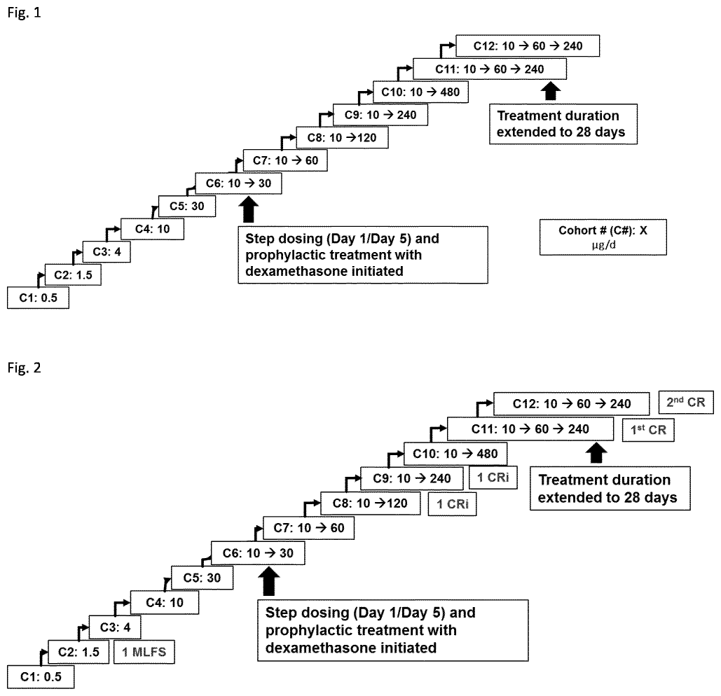

Methods (see FIG. 1): This was a phase 1 dose escalation study evaluating CD33×CD3 bispecific antibody construct as a continuous IV infusion in patients with R/R AML, with single-patient cohorts for the first 3 doses and then subsequently 3-6 patients per cohort (NCT02520427). Response was per revised IWG criteria with the addition of complete response (CR) with partial hematologic recovery. After completing the first cycle without dose-limiting toxicity (DLT), up to 5 additional cycles could be given for clinical benefit. After the 30 μg/day (d) cohort, risk mitigation measures for cytokine release syndrome (CRS) were put in place, including step-dosing and pretreatment with a single dose of corticosteroids. The modified treatment regimen consisted of an initial run-in dose of 10 μg/d×4d followed by the target dose. A 2-step regimen was then tested, ie 10 μg/d, 60 μg/d, and then the target dose, for a treatment duration of 14d or 28d, followed by 1-4 weeks off treatment.

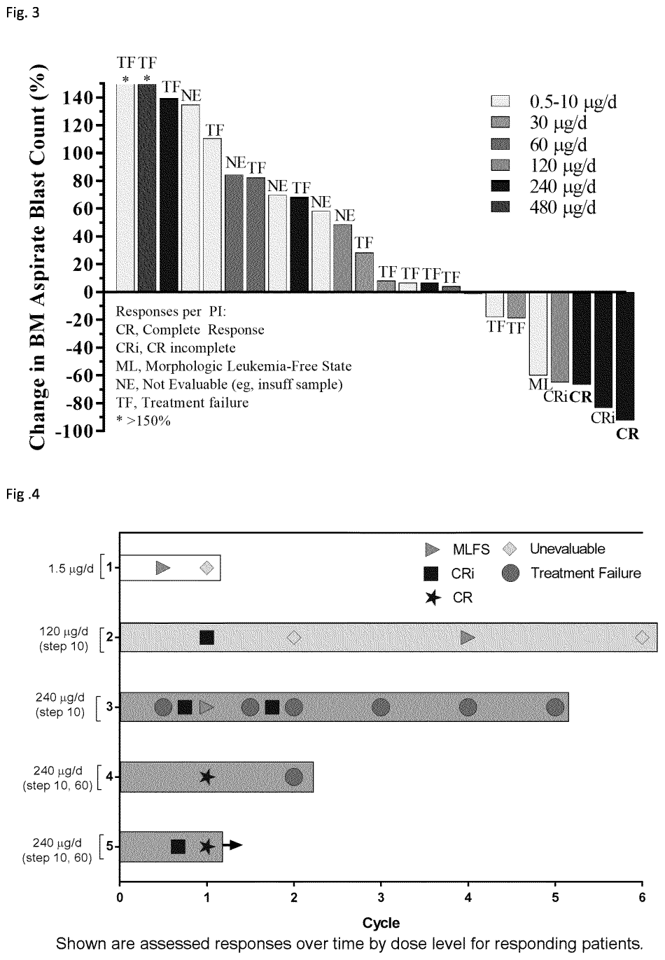

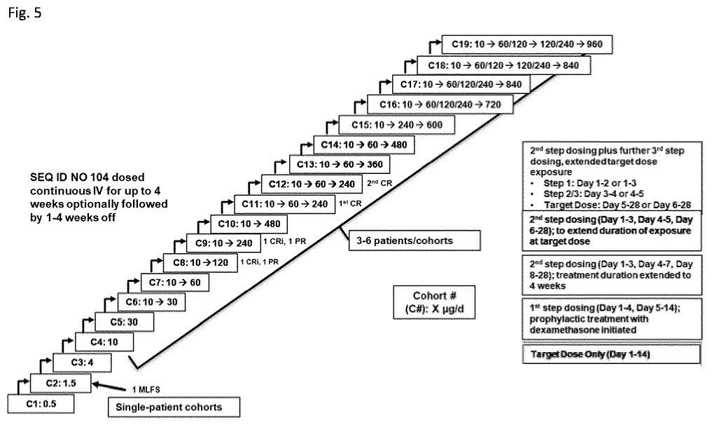

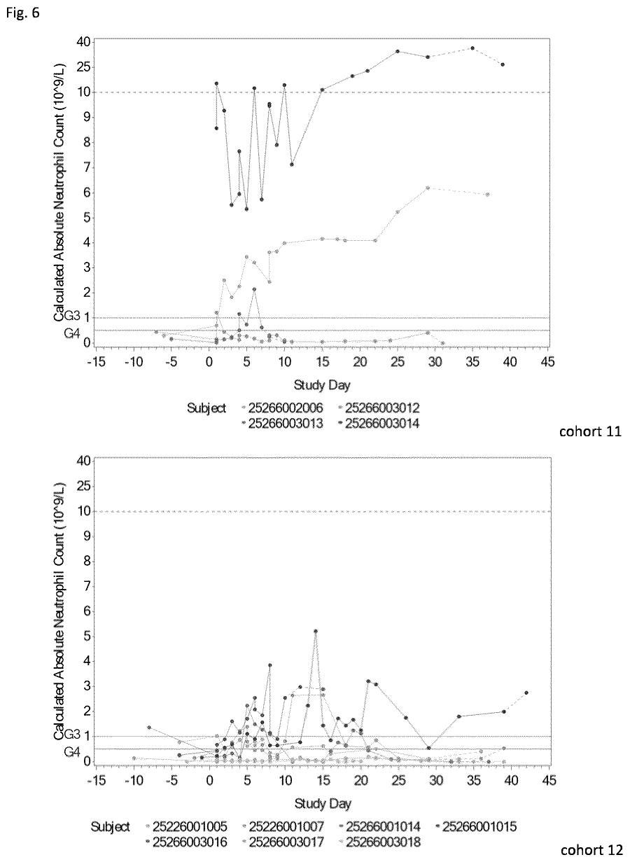

Results (see Table 1 to 3, FIGS. 2 to 4): 35 patients had enrolled in 12 dose cohorts with a target dose range of 0.5-480 μg/d in this ongoing study. Over half ( 20/35, 57%) of patients were male and the median age was 58 (range: 18-80) years; 14/35 (40%) have previously received a stem cell transplant. Median AML disease duration at baseline was 1.3 (range: 0.3-9.6) years, median proportion of blasts at baseline was 37% (range: 3%-95%), and the median # of prior treatments was 4 (range: 1-15). Median baseline ANC was 0.2 (range: 0-8.6)×10⁹/L.

Patients received a median of 1 (range: 1-6) cycle with CD33×CD3 bispecific antibody construct; 31/35 (89%) patients discontinued treatment for disease progression (n=24), adverse events (AEs; n=5, 2 treatment-related), and patient request (n=2). One patient completed the maximum of 6 cycles allowed and 3 patients are still receiving study drug. Serious AEs (SAEs) were seen in 23/35 (66%) patients (treatment-related in 15 patients); SAEs seen in >1 patient included CRS (n=11), febrile neutropenia (n=6), pneumonia (n=4), leukopenia (n=3), thrombocytopenia (n=2), and subdural hematoma (n=2); 1 patient died on study due to AML progression (not treatment-related). One patient each in the 10 μg/d and 30 μg/d cohorts (no lead-in) experienced severe CRS; CRS signs and symptoms resolved in 1d with corticosteroids, vasopressors, and IV fluids, and interruption of CD33×CD3 bispecific antibody construct. There were DLTs of grade 2 CRS and grade 4 ventricular fibrillation with a target dose of 480 μg/d; the target dose was then decreased to 240 μg/d.

Two patients had CRs at a target dose of 240 μg/d (lead-in of 10 μg/d→60 μg/d); 1 patient each at target doses of 120 μg/d and 240 μg/d had a CRi and 1 patient who received 1.5 μg/d had a morphologic leukemia-free state (MLFS, <5% blasts, no hematologic recovery). One patient with a CR had bone marrow blasts decrease from ˜5%-10% (estimated as patchy disease) to 2.5% by d29 by flow cytometry, with no morphologic evidence of residual AML and normo- to hypercellular marrow and recovery of peripheral blood counts. The second CR patient had blasts decrease from 40% to 3% with recovery of peripheral blood counts by d42 after receiving one cycle of CD33×CD3 bispecific antibody construct. Correlative data will be presented.

Conclusions: Preliminary data of CD33×CD3 bispecific antibody construct dosed up to 480 μg/d provide encouraging early evidence of tolerability and anti-leukemic activity in heavily pre-treated patients with R/R AML. Expected CRS was mitigated through step-up dosing, corticosteroid pretreatment, IV fluids, tocilizumab, and drug interruption if needed; most patients had short periods of CRS which responded well to treatment. A 2-step approach will be used in the future to quickly achieve the target dose and optimize clinical response. To date, 2 CRs and 2 CR is have been observed at target doses of 120 and 240 μg/d. As nearly all patients were substantially cytopenic at baseline, it is challenging to evaluate the impact of CD33×CD3 bispecific antibody construct on cytopenias. Of note, both CR patients had a complete recovery of blood counts after one cycle of treatment. These promising data validate the use of the BITE® platform to target CD33.

### Example 3

The objective of this study was to shorten the dose limiting toxicity (DLT) window for CD33×CD3 bispecific antibody constructs. The DLT window applied in the other examples comprised the treatment cycle (e.g. 4 weeks) and an additional treatment free period of e.g. 2 weeks after the end of the cycle. This study focused on the actual treatment period and the 2-week post cycle period from the DLT window was removed after the first cycle.

Conclusions: These changes improved the benefit:risk profile of SEQ ID NO: 104 for enrolled and future subjects. Study 20120252 is designed to evaluate the safety, tolerability, pharmacokinetics, pharmacodynamics, and efficacy of SEQ ID NO: 104 in subjects with relapsed or refractory acute myeloid leukemia (AML), and is currently being conducted in Germany, the Netherlands, and the United States (US).

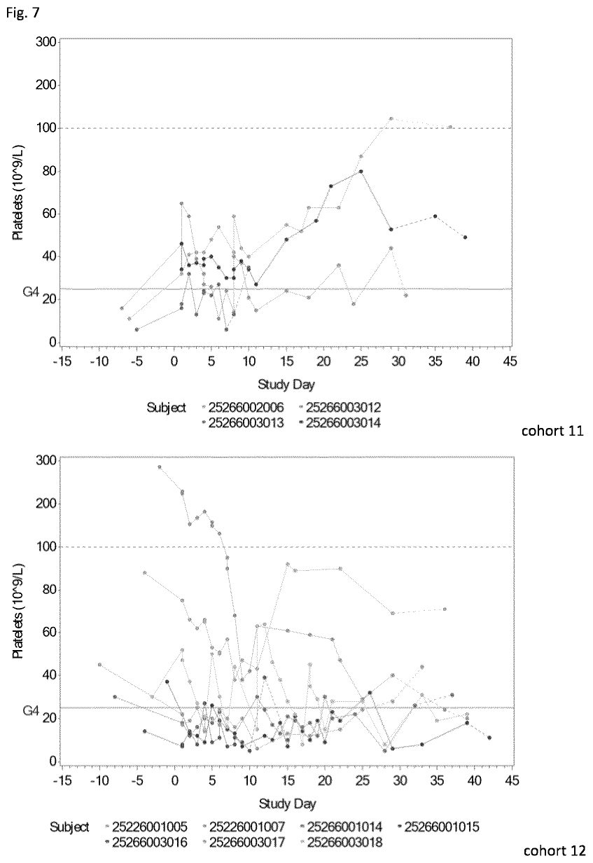

Rationale for Update of DLT Window: The 2-week off-drug period was previously added to the DLT window to monitor the dynamics of peripheral blood cell recovery in patients achieving complete response (CR). Because mature myeloid cells and myeloid progenitors express CD33, the concern was that the treatment with SEQ ID NO: 104 may result in an inhibition of myeloid lineage reflected by persistent myelosuppression. To investigate the effects of SEQ ID NO: 104 on myeloid lineage, absolute neutrophil counts (ANC) were analyzed in all treated subjects. Subjects enrolled in cohorts 11, 12 and 13 were treated with the two dose-step schedule (i.e. Schedule 3: first dose step of 10 μg/day followed by a second dose step of 60 μg/day followed by the target dose). Review of individual subject results showed that most subjects treated in Cohorts 11-13 had Grade 3-4 neutropenia at baseline—a reflection of the nature of the underlying disease (FIG. 6). Similar results were obtained for subjects treated on Schedule 1 (Cohorts 1-5, target dose escalation without a dose step) and Schedule 2 (Cohorts 6-10, one dose step of 10 μg/day followed by a target dose) (data not shown).

Most subjects remained neutropenic but some subjects showed an improvement of neutrophil counts during the SEQ ID NO: 104 treatment. The review of data from each cohort did not show any clinically meaningful changes in neutrophil counts between the baseline and the end of cycle treatment. Furthermore, the majority of subjects treated in Cohorts 11-13 did not complete the last 2 weeks of the DLT period (i.e. the off-drug period) due to disease progression.

However, 2 subjects treated on Cohorts 11 and 12 achieved a CR with complete hematologic recovery suggesting that SEQ ID NO: 104 may not interfere with normal hematopoiesis.

Absolute neutrophil counts in peripheral blood of patients treated in cohorts 11 (240 μg), 12 (240 μg) and 13 (360 μg) during the DLT window are shown (FIG. 6). Mean±SE is shown. G4 line (lower line) and G3 line (upper line) show grade 4 and 3 neutropenia by CTCAE

In addition to analysis of the clinical data in the FIH study, the potential effects of SEQ ID NO: 104 by continuous intravenous (CIV) or subcutaneous (SC) administration on circulating monocytes and neutrophils was evaluated in a Good Laboratory Practice (GLP)-compliant 28-day repeat dose toxicology study in cynomolgus monkeys (Study 119422, SEQ ID NO: 104: 28-Day Continuous Intravenous Infusion or Subcutaneous Toxicology Study in the Cynomolgus Monkey). The hematology assessment was performed pre-study (twice) and on day 1 (4 hrs), day 2, day 10, and day 29. Decreases in monocytes in the 3, 10, and 30 μg/kg/day CIV and 25 ug/kg/day SC dose groups and/or neutrophils in the 10 and 30 μg/kg/day CIV and 25 μg/kg/day SC dose groups were detected on days 1 or 2. The decreases were graded as “mild to marked” on a 5-point scale (minimal, mild, moderate, marked, severe). The number of circulating monocytes advantageously recovered to pre-dose values by day 29 and the number of circulating neutrophils recovered to pre-dose values by day 29. Therefore, SEQ ID NO: 104-induced decreases in circulating myeloid cells were only transient as animals in the 3, 10 and 30 μg/kg/day CIV groups that maintained drug exposure through day 28.

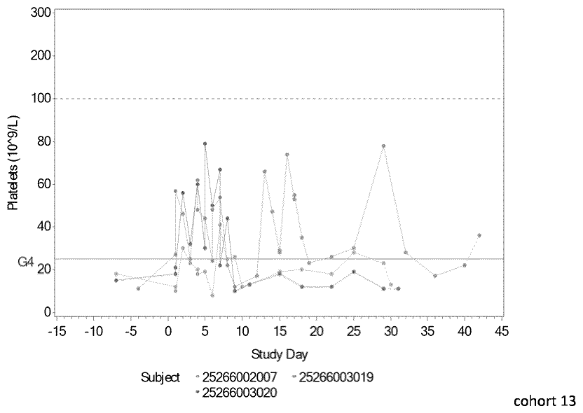

While platelets are CD33-negative and are not directly targeted by SEQ ID NO: 104, they originate from myeloid progenitors that express CD33. Thus, platelet counts in peripheral blood were assessed to evaluate the potential inhibitory effect of SEQ ID NO: 104 on common myeloid progenitors. The analysis of platelet numbers in subjects treated in Cohorts 11-13 showed that the majority of subjects were thrombocytopenic at baseline. Review of platelet dynamics during treatment with SEQ ID NO: 104 did not reveal any clinically meaningful changes in the platelet numbers compared to baseline (FIG. 7). This finding is in line with toxicology study results showing that administration of 10 μg/kg/day SEQ ID NO: 104 by CIV route resulted only in a transient and minimal decrease in platelet counts in cynomolgus monkeys.

Platelet counts in peripheral blood of patients treated in cohorts 11 (240 μg), 12 (240 μg) and 13 (360 μg) during the DLT window are shown. Mean±SE is shown. G4 line (lower line) and G3 line (upper line) show grade 4 and 3 neutropenia by CTCAE.

CRS has been defined as an on-target toxicity in Study 20120252. To understand the dynamics of onset and resolution of CRS, data available for subjects in Cohorts 11, 12 and 13 treated on the current schedule were analyzed. The onset of all CRS events occurred within first 10 days of the SEQ ID NO: 104 dose, i.e. dose steps and/or target dose or shortly thereafter. A similar dynamic has been observed in subjects treated in cohorts 1-10 (data not shown). This finding is in line with the current knowledge that CRS is an acute toxicity observed early following a dose administration or a dose increase. No delayed CRS has been observed in subjects to date. Hence, the proposed DLT window (4 weeks total with at least 14 days on the target dose) provides sufficient time for CRS to resolve. Additionally, under the circumstance that a Grade 2-3 CRS does not resolve within 7 days, it is classified as a DLT per protocol.

The onset and resolution of CRS in patients treated in cohorts 11 (240 μg), 12 (240 μg) & 13 (360 μg) during the DLT window are shown. On the left, data show the initial doses and the restart of doses leading to the restart of the DLT window. On the right, data are normalized to the beginning of the final DLT window.

In summary, absolute neutrophil counts in exemplary cohorts 11 to 13 of Study 20120252 showed that subjects were myelosuppressed at baseline and no decrease in neutrophil counts was observed post-treatment with SEQ ID NO: 104. This is in line with studies in cynomolgus monkeys where the effect of SEQ ID NO: 104 on myeloid lineage occurred early during drug exposure and was transient in nature. Additionally, the onset of CRS in cohorts 11 to 13 of Study 20120252 occurred within the first 10 days of given dose steps and a target dose of SEQ ID NO: 104, without any evidence of delayed toxicity. Based on these findings, the DLT window can be shortened to a standard of 4 weeks (with at least 14 days on the target dose) allowing for monitoring the onset of CRS and its resolutions, efficient intra-subject escalation, and overall patient safety.

### Example 4

### Evaluation of the Avoidance or Attenuation of Side Effects (CRS Events)

Of 46 subjects having been treated in exemplary Cohorts 1-14 (target doses 0.5 to 480 μg), twenty-nine of the 46 subjects (63%) treated experienced some event of CRS. The 29 subjects experienced 56 events of CRS the severity of which were 29/56 (52%) Grade 1, 21/56 (37.5%) Grade 2, 4/56 (7%) Grade 3 and 2/56 (3.5%) Grade 4. There were no Grade 5 events of CRS. Drug was interrupted for 19/56 (34%) and withdrawn for only 1/56 (2%). No action was taken with SEQ ID NO: 104 for 36/56 (64%) of subjects. Hence, side effect CRS of the highest grade could be avoided completely and the higher grades 3 and 4 attenuated to infrequent single digit occurrences. A treatment interruption could be avoided in the majority of treated patients and ensure continuous effective dose administration to treat high patients suffering from highly progressive r/r AML.

| SEQ | | | |
|---|---|---|---|
| ID NO. | DESIGNATION | SOURCE | SEQUENCE |
| 1 | Human | human | QDGNEEMGGITQTPYKVSISGTTVILTCPQYPGSEILWQHNDKNIGGDEDDK |
| | CD3ϵ extracellular | | NIGSDEDHLSLKEFSELEQSGYYVCYPRGSKPEDANFYLYLRARVCENCMEM |
| | domain | | D |
| 2 | Human CD3ϵ 1-27 | human | QDGNEEMGGITQTPYKVSISGTTVILT |
| 3 | Callithrix jacchus CD3ϵ | Callithrix | QDGNEEMGDTTQNPYKVSISGTTVTLTCPRYDGHEIKWLVNSQNKEGHEDHL |
| | extracellular domain | jacchus | LLEDFSEMEQSGYYACLSKETPAEEASHYLYLKARVCENCVEVD |
| 4 | Callithrix jacchus CD3ϵ | Callithrix | QDGNEEMGDTTQNPYKVSISGTTVTLT |
| | 1-27 | jacchus | |
| 5 | Saguinus oedipus | Saguinus | QDGNEEMGDTTQNPYKVSISGTTVTLTCPRYDGHEIKWLVNSQNKEGHEDHL |
| | CD3ϵ extracellular | oedipus | LLEDFSEMEQSGYYACLSKETPAEEASHYLYLKARVCENCVEVD |
| | domain | | |
| 6 | Saguinus oedipus | Saguinus | QDGNEEMGDTTQNPYKVSISGTTVTLT |
| | CD3ϵ 1-27 | oedipus | |
| 7 | Saimiri sciureus CD3ϵ | Saimiri | QDGNEEIGDTTQNPYKVSISGTTVTLTCPRYDGQEIKWLVNDQNKEGHEDHL |
| | extracellular domain | sciureus | LLEDFSEMEQSGYYACLSKETPTEEASHYLYLKARVCENCVEVD |
| 8 | Saimiri sciureus CD3ϵ | Saimiri | QDGNEEIGDTTQNPYKVSISGTTVTLT |
| | 1-27 | sciureus | |
| 9 | CD33 VH of AH3 | artificial | QVQLVQSGAEVKKPGESVKVSCKASGYTFTNYGMNWVRQAPGQGLEWMGWIN |
| | | | TYTGEPTYADDFKGRVTMSSDTSTSTAYLEINSLRSDDTAIYYCARWSWSDG |
| | | | YYVYFDYWGQGTTVTVSS |
| 10 | CD33 HCDR1 of AH3 | artificial | NYGMN |
| 11 | CD33 HCDR2 of AH3 | artificial | WINTYTGEPTYADDFKG |
| 12 | CD33 HCDR3 of AH3 | artificial | WSWSDGYYVYFDY |
| 13 | CD33 VL of AH3 | artificial | DIVMTQSPDSLTVSLGERTTINCKSSQSVLDSSKNKNSLAWYQQKPGQPPKL |
| | | | LLSWASTRESGIPDRFSGSGSGTDFTLTIDSLQPEDSATYYCQQSAHFPITF |
| | | | GQGTRLEIK |
| 14 | CD33 LCDR1 of AH3 | artificial | KSSQSVLDSSKNKNSLA |
| 15 | CD33 LCDR2 of AH3 | artificial | WASTRES |
| 16 | CD33 LCDR3 of AH3 | artificial | QQSAHFPIT |
| 17 | CD33 CD33 HL of AH3 | artificial | QVQLVQSGAEVKKPGESVKVSCKASGYTFTNYGMNWVRQAPGQGLEWMGWIN |
| | | | TYTGEPTYADDFKGRVTMSSDTSTSTAYLEINSLRSDDTAIYYCARWSWSDG |
| | | | YYVYFDYWGQGTTVTVSSGGGGSGGGGSGGGGSDIVMTQSPDSLTVSLGERT |
| | | | TINCKSSQSVLDSSKNKNSLAWYQQKPGQPPKLLLSWASTRESGIPDRFSGS |
| | | | GSGTDFTLTIDSLQPEDSATYYCQQSAHFPITFGQGTRLEIK |
| 18 | CD33 AH3 HL × H2C | artificial | QVQLVQSGAEVKKPGESVKVSCKASGYTFTNYGMNWVRQAPGQGLEWMGWIN |
| | HL | | TYTGEPTYADDFKGRVTMSSDTSTSTAYLEINSLRSDDTAIYYCARWSWSDG |
| | | | YYVYFDYWGQGTTVTVSSGGGGSGGGGSGGGGSDIVMTQSPDSLTVSLGERT |
| | | | TINCKSSQSVLDSSKNKNSLAWYQQKPGQPPKLLLSWASTRESGIPDRFSGS |
| | | | GSGTDFTLTIDSLQPEDSATYYCQQSAHFPITFGQGTRLEIKSGGGGSEVQL |
| | | | VESGGGLVQPGGSLKLSCAASGFTFNKYAMNWVRQAPGKGLEWVARIRSKYN |
| | | | NYATYYADSVKDRFTISRDDSKNTAYLQMNNLKTEDTAVYYCVRHGNFGNSY |
| | | | ISYWAYWGQGTLVTVSSGGGGSGGGGSGGGGSQTVVTQEPSLTVSPGGTVTL |
| | | | TCGSSTGAVTSGYYPNWVQQKPGQAPRGLIGGTKFLAPGTPARFSGSLLGGK |
| | | | AALTLSGVQPEDEAEYYCALWYSNRWVFGGGTKLTVL |
| 19 | CD33 AH3 HL × F12Q | artificial | QVQLVQSGAEVKKPGESVKVSCKASGYTFTNYCMNWVRQAPCQGLEWMGWIN |
| | HL | | TYTGEPTYADDFKGRVTMSSDTSTSTAYLEINSLRSDDTAIYYCARWSWSDG |
| | | | YYVYFDYWGQGTTVTVSSGGGGSGGGGSGGGGSDIVMTQSPDSLTVSLGERT |
| | | | TINCKSSQSVLDSSKNKNSLAWYQQKPGQPPKLLLSWASTRESGIPDRFSGS |
| | | | GSGTDFTLTIDSLQPEDSATYYCQQSAHFPITFGQGTRLEIKSGGGGSEVQL |
| | | | VESGGGLVQPGGSLKLSCAASGFTFNSYAMNWVRQAPGKGLEWVARIRSKYN |
| | | | NYATYYADSVKGRFTISRDDSKNTAYLQMNNLKTEDTAVYYCVRHGNFGNSY |
| | | | VSWWAYWGQGTLVTVSSGGGGSGGGGSGGGGSQTVVTQEPSLTVSPGGTVTL |
| | | | TCGSSTCAVTSCNYPNWVQQKPGQAPRGLIGGTKFLAPCTPARFSCSLLGGK |
| | | | AALTLSGVQPEDEAEYYCVLWYSNRWVFGGGTKLTVL |
| 20 | CD33 AH3 HL × I2C | artificial | QVQLVQSGAEVKKPGESVKVSCKASGYTFTNYGMNWVRQAPGQGLEWMGWIN |
| | HL | | TYTGEPTYADDFKGRVTMSSDTSTSTAYLEINSLRSDDTAIYYCARWSWSDG |
| | | | YYVYFDYWGQGTTVTVSSGGGGSGGGGSGGGGSDIVMTQSPDSLTVSLGERT |
| | | | TINCKSSQSVLDSSKNKNSLAWYQQKPGQPPKLLLSWASTRESGIPDRFSGS |
| | | | GSGTDFTLTIDSLQPEDSATYYCQQSAHFPITFGQGTRLEIKSGGGGSEVQL |
| | | | VESGGGLVQPGGSLKLSCAASGFTFNKYAMNWVRQAPGKGLEWVARIRSKYN |
| | | | NYATYYADSVKDRFTISRDDSKNTAYLQMNNLKTEDTAVYYCVRHGNFGNSY |
| | | | ISYWAYWGQGTLVTVSSGGGGSGGGGSGGGGSQTVVTQEPSLTVSPGGTVTL |
| | | | TCGSSTGAVTSGNYPNWVQQKPGQAPRGLIGGTKFLAPGTPARFSGSLLGGK |
| | | | AALTLSGVQPEDEAEYYCVLWYSNRWVFGGGTKLTVL |
| 21 | CD33 VH of AF5 | artificial | QVQLVQSGAEVKKPGASVKVSCKASGYTFTNYGMNWVKQAPGQGLKWMGWIN |
| | | | TYTGEPTYADDFKGRVTMTSDTSTSTAYLELHNLRSDDTAVYYCARWSWSDG |
| | | | YYVYFDYWGQGTTVTVSS |
| 22 | CD33 HCDR1 of AF5 | artificial | NYGMN |
| 23 | CD33 HCDR2 of AF5 | artificial | WINTYTGEPTYADDFKG |
| 24 | CD33 HCDR3 of AF5 | artificial | WSWSDGYYVYFDY |
| 25 | CD33 VL of AF5 | artificial | DIVMTQSPDSLTVSLGERTTINCKSSQSVLDSSKNKNSLAWYQQKPGQPPKL |
| | | | LLSWASTRESGIPDRFSGSGSGTDFTLTIDSLQPEDSATYYCQQSAHFPITF |
| | | | GQGTRLEIK |
| 26 | CD33 LCDR1 of AF5 | artificial | KSSQSVLDSSKNKNSLA |
| 27 | CD33 LCDR2 of AF5 | artificial | WASTRES |
| 28 | CD33 LCDR3 of AF5 | artificial | QQSAHFPIT |
| 29 | CD33 HL of AF5 | artificial | QVQLVQSGAEVKKPGASVKVSCKASGYTFTNYCMNWVKQAPGQGLKWMGWIN |
| | | | TYTGEPTYADDFKGRVTMTSDTSTSTAYLELHNLRSDDTAVYYCARWSWSDG |
| | | | YYVYFDYWGQGTTVTVSSGGGGSGGGGSGGGGSDIVMTQSPDSLTVSLGERT |
| | | | TINCKSSQSVLDSSKNKNSLAWYQQKPGQPPKLLLSWASTRESGIPDRFSGS |
| | | | GSGTDFTLTIDSLQPEDSATYYCQQSAHFPITFGQGTRLEIK |
| 30 | CD33 AF5 HL × H2C | artificial | QVQLVQSGAEVKKPGASVKVSCKASGYTFTNYGMNWVKQAPGQGLKWMGWIN |
| | HL | | TYTGEPTYADDFKGRVTMTSDTSTSTAYLELHNLRSDDTAVYYCARWSWSDG |
| | | | YYVYFDYWGQGTTVTVSSGGGGSGGGGSGGGGSDIVMTQSPDSLTVSLGERT |
| | | | TINCKSSQSVLDSSKNKNSLAWYQQKPGQPPKLLLSWASTRESGIPDRFSGS |
| | | | GSGTDFTLTIDSLQPEDSATYYCQQSAHFPITFGQGTRLEIKSGGGGSEVQL |
| | | | VESGGGLVQPGGSLKLSCAASGFTFNKYAMNWVRQAPGKGLEWVARIRSKYN |
| | | | NYATYYADSVKDRFTISRDDSKNTAYLQMNNLKTEDTAVYYCVRHGNFGNSY |
| | | | ISYWAYWGQGTLVTVSSGGGGSGGGGSGGGGSQTVVTQEPSLTVSPGGTVTL |
| | | | TCGSSTGAVTSGYYPNWVQQKPGQAPRGLIGGTKFLAPGTPARFSGSLLGGK |
| | | | AALTLSGVQPEDEAEYYCALWYSNRWVFGGGTKLTVL |
| 31 | CD33 AF5 HL × F12Q | artificial | QVQLVQSGAEVKKPGASVKVSCKASGYTFTNYGMNWVKQAPGQGLKWMGWIN |
| | HL | | TYTGEPTYADDFKGRVTMTSDTSTSTAYLELHNLRSDDTAVYYCARWSWSDG |
| | | | YYVYFDYWGQGTTVTVSSGGGGSGGGGSGGGGSDIVMTQSPDSLTVSLGERT |
| | | | TINCKSSQSVLDSSKNKNSLAWYQQKPGQPPKLLLSWASTRESGIPDRFSGS |
| | | | GSGTDFTLTIDSLQPEDSATYYCQQSAHFPITFGQGTRLEIKSGGGGSEVQL |
| | | | VESGGGLVQPGGSLKLSCAASGFTFNSYAMNWVRQAPGKGLEWVARIRSKYN |
| | | | NYATYYADSVKGRFTISRDDSKNTAYLQMNNLKTEDTAVYYCVRHGNFGNSY |
| | | | VSWWAYWGQGTLVTVSSGGGGSGGGGSGGGGSQTVVTQEPSLTVSPGGTVTL |
| | | | TCGSSTGAVTSGNYPNWVQQKPGQAPRGLIGGTKFLAPGTPARFSGSLLGGK |
| | | | AALTLSGVQPEDEAEYYCVLWYSNRWVFGGGTKLTVL |
| 32 | CD33 AF5 HL × I2C | artificial | QVQLVQSGAEVKKPGASVKVSCKASGYTFTNYGMNWVKQAPGQGLKWMGWIN |
| | HL | | TYTGEPTYADDFKGRVTMTSDTSTSTAYLELHNLRSDDTAVYYCARWSWSDG |
| | | | YYVYFDYWGQGTTVTVSSGGGGSGGGGSGGGGSDIVMTQSPDSLTVSLGERT |
| | | | TINCKSSQSVLDSSKNKNSLAWYQQKPGQPPKLLLSWASTRESGIPDRFSGS |
| | | | GSGTDFTLTIDSLQPEDSATYYCQQSAHFPITFGQGTRLEIKSGGGGSEVQL |
| | | | VESGGGLVQPGGSLKLSCAASGFTFNKYAMNWVRQAPGKGLEWVARIRSKYN |
| | | | NYATYYADSVKDRFTISRDDSKNTAYLQMNNLKTEDTAVYYCVRHGNFGNSY |
| | | | ISYWAYWGQGTLVTVSSGGGGSGGGGSGGGGSQTVVTQEPSLTVSPGGTVTL |
| | | | TCGSSTGAVTSGNYPNWVQQKPGQAPRGLIGGTKFLAPGTPARFSGSLLGGK |
| | | | AALTLSGVQPEDEAEYYCVLWYSNRWVFGGGTKLTVL |
| 33 | CD33 VH of AC8 | artificial | QVQLVQSGAEVKKPGESVKVSCKASGYTFTNYGMNWVKQAPGQGLKWMGWIN |
| | | | TYTGEPTYADDFKGRVTMTTDTSTSTAYMEIRNLRNDDTAVYYCARWSWSDG |
| | | | YYVYFDYWGQGTTVTVSS |
| 34 | CD33 HCDR1 of AC8 | artificial | NYGMN |
| 35 | CD33 HCDR2 of AC8 | artificial | WINTYTGEPTYADDFKG |
| 36 | CD33 HCDR3 of AC8 | artificial | WSWSDGYYVYFDY |
| 37 | CD33 VL of AC8 | artificial | DIVMTQSPDSLTVSLGERTTINCKSSQSVLDSSKNKNSLAWYQQKPGQPPKL |
| | | | LLSWASTRESGIPDRFSGSGSGTDFTLTIDSLQPEDSATYYCQQSAHFPITF |
| | | | GQGTRLEIK |
| 38 | CD33 LCDR1 of AC8 | artificial | KSSQSVLDSSKNKNSLA |
| 39 | CD33 LCDR2 of AC8 | artificial | WASTRES |
| 40 | CD33 LCDR3 of AC8 | artificial | QQSAHFPIT |
| 41 | CD33 HL of AC8 | artificial | QVQLVQSGAEVKKPGESVKVSCKASGYTFTNYGMNWVKQAPGQGLKWMGWIN |
| | | | TYTGEPTYADDFKGRVTMTTDTSTSTAYMEIRNLRNDDTAVYYCARWSWSDG |
| | | | YYVYFDYWGQGTTVTVSSGGGGSGGGGSGGGGSDIVMTQSPDSLTVSLGERT |
| | | | TINCKSSQSVLDSSKNKNSLAWYQQKPGQPPKLLLSWASTRESGIPDRFSGS |
| | | | GSGTDFTLTIDSLQPEDSATYYCQQSAHFPITFGQGTRLEIK |
| 42 | CD33 AC8 HL × H2C | artificial | QVQLVQSGAEVKKPGESVKVSCKASGYTFTNYGMNWVKQAPGQGLKWMGWIN |
| | HL | | TYTGEPTYADDFKGRVTMTTDTSTSTAYMEIRNLRNDDTAVYYCARWSWSDG |
| | | | YYVYFDYWGQGTTVTVSSGGGGSGGGGSGGGGSDIVMTQSPDSLTVSLGERT |
| | | | TINCKSSQSVLDSSKNKNSLAWYQQKPGQPPKLLLSWASTRESGIPDRFSGS |
| | | | GSGTDFTLTIDSLQPEDSATYYCQQSAHFPITFGQGTRLEIKSGGGGSEVQL |
| | | | VESGGGLVQPCCSLKLSCAASGFTFNKYAMNWVRQAPGKGLEWVARIRSKYN |
| | | | NYATYYADSVKDRFTISRDDSKNTAYLQMNNLKTEDTAVYYCVRHGNFGNSY |
| | | | ISYWAYWGQGTLVTVSSGGGGSGGGGSGGGGSQTVVTQEPSLTVSPGGTVTL |
| | | | TCGSSTGAVTSGYYPNWVQQKPGQAPRGLIGGTKFLAPGTPARFSGSLLGGK |
| | | | AALTLSGVQPEDEAEYYCALWYSNRWVFGGGTKLTVL |
| 43 | CD33 AC8 HL × F12Q | artificial | QVQLVQSGAEVKKPGESVKVSCKASCYTFTNYGMNWVKQAPGQGLKWMCWIN |
| | HL | | TYTGEPTYADDFKGRVTMTTDTSTSTAYMEIRNLRNDDTAVYYCARWSWSDG |
| | | | YYVYFDYWGQGTTVTVSSGGGGSGGGGSGGGGSDIVMTQSPDSLTVSLGERT |
| | | | TINCKSSQSVLDSSKNKNSLAWYQQKPGQPPKLLLSWASTRESGIPDRFSGS |
| | | | GSGTDFTLTIDSLQPEDSATYYCQQSAHFPITFGQGTRLEIKSGGGGSEVQL |
| | | | VESGGGLVQPGGSLKLSCAASGFTFNSYAMNWVRQAPGKGLEWVARIRSKYN |
| | | | NYATYYADSVKGRFTISRDDSKNTAYLQMNNLKTEDTAVYYCVRHGNFGNSY |
| | | | VSWWAYWGQGTLVTVSSGGGGSGGGGSGGGGSQTVVTQEPSLTVSPGGTVTL |
| | | | TCGSSTGAVTSGNYPNWVQQKPGQAPRGLIGGTKFLAPGTPARFSGSLLGGK |
| | | | AALTLSGVQPEDEAEYYCVLWYSNRWVFGGGTKLTVL |
| 44 | CD33 AC8 HL × I2C | artificial | QVQLVQSGAEVKKPGESVKVSCKASGYTFTNYGMNWVKQAPGQGLKWMGWIN |
| | HL | | TYTGEPTYADDFKGRVTMTTDTSTSTAYMEIRNLRNDDTAVYYCARWSWSDG |
| | | | YYVYFDYWGQGTTVTVSSGGGGSGGGGSGGGGSDIVMTQSPDSLTVSLGERT |
| | | | TINCKSSQSVLDSSKNKNSLAWYQQKPGQPPKLLLSWASTRESGIPDRFSGS |
| | | | GSGTDFTLTIDSLQPEDSATYYCQQSAHFPITFGQGTRLEIKSGGGGSEVQL |
| | | | VESGGGLVQPGGSLKLSCAASGFTFNKYAMNWVRQAPGKGLEWVARIRSKYN |
| | | | NYATYYADSVKDRFTISRDDSKNTAYLQMNNLKTEDTAVYYCVRHGNFGNSY |
| | | | ISYWAYWGQGTLVTVSSGGGGSGGGGSGGGGSQTVVTQEPSLTVSPGGTVTL |
| | | | TCGSSTGAVTSGNYPNWVQQKPGQAPRGLIGGTKFLAPGTPARFSGSLLGGK |
| | | | AALTLSGVQPEDEAEYYCVLWYSNRWVFGGGTKLTVL |
| 45 | CD33 VH of AH11 | artificial | QVQLVQSGAEVKKPGESVKVSCKASGYTFTNYGMNWVKQAPGQGLKWMGWIN |
| | | | TYTGEPTYADDFKGRVTMTSDTSTSTAYMEISSLRSDDTAVYYCARWSWSDG |
| | | | YYVYFDYWGQGTTVTVSS |
| 46 | CD33 HCDR1 of AH11 | artificial | NYGMN |
| 47 | CD33 HCDR2 of AH11 | artificial | WINTYTGEPTYADDFKG |
| 48 | HCDR3 | artificial | WSWSDGYYVYFDY |
| 49 | CD33 VL of AH11 | artificial | DIVMTQSPDSLTVSLGERTTINCKSSQSVLDSSKNKNSLAWYQQKPGQPPKL |
| | | | LLSWASTRESGIPDRFSGSGSGTDFTLTIDSLQPEDSATYYCQQSAHFPITF |
| | | | GQGTRLEIK |
| 50 | CD33 LCDR1 of AH11 | artificial | KSSQSVLDSSKNKNSLA |
| 51 | CD33 LCDR2 of AH11 | artificial | WASTRES |
| 52 | CD33 LCDR3 of AH11 | artificial | QQSAHFPIT |
| 53 | CD33 HL of AH11 | artificial | QVQLVQSGAEVKKPGESVKVSCKASGYTFTNYGMNWVKQAPGQGLKWMGWIN |
| | | | TYTGEPTYADDFKGRVTMTSDTSTSTAYMEISSLRSDDTAVYYCARWSWSDG |
| | | | YYVYFDYWGQGTTVTVSSGGGGSGGGGSGGGGSDIVMTQSPDSLTVSLGERT |
| | | | TINCKSSQSVLDSSKNKNSLAWYQQKPGQPPKLLLSWASTRESGIPDRFSGS |
| | | | GSGTDFTLTIDSLQPEDSATYYCQQSAHFPITFGQGTRLEIK |
| 54 | CD33 AH11 HL × H2C | artificial | QVQLVQSGAEVKKPGESVKVSCKASGYTFTNYGMNWVKQAPGQGLKWMGWIN |
| | HL | | TYTGEPTYADDFKGRVTMTSDTSTSTAYMEISSLRSDDTAVYYCARWSWSDG |
| | | | YYVYFDYWGQGTTVTVSSGGGGSGGGGSGGGGSDIVMTQSPDSLTVSLGERT |
| | | | TINCKSSQSVLDSSKNKNSLAWYQQKPGQPPKLLLSWASTRESGIPDRFSGS |
| | | | GSGTDFTLTIDSLQPEDSATYYCQQSAHFPITFGQGTRLEIKSGGGGSEVQL |
| | | | VESGGGLVQPGGSLKLSCAASGFTFNKYAMNWVRQAPGKGLEWVARIRSKYN |
| | | | NYATYYADSVKDRFTISRDDSKNTAYLQMNNLKTEDTAVYYCVRHGNFGNSY |
| | | | ISYWAYWGQGTLVTVSSGGGGSGGGGSGGGGSQTVVTQEPSLTVSPGGTVTL |
| | | | TCGSSTGAVTSGYYPNWVQQKPGQAPRGLIGGTKFLAPGTPARFSGSLLGGK |
| | | | AALTLSGVQPEDEAEYYCALWYSNRWVFGGGTKLTVL |
| 55 | CD33 AH11 HL × F12Q | artificial | QVQLVQSGAEVKKPGESVKVSCKASGYTFTNYGMNWVKQAPGQGLKWMGWIN |
| | HL | | TYTGEPTYADDFKGRVTMTSDTSTSTAYMEISSLRSDDTAVYYCARWSWSDG |
| | | | YYVYFDYWGQGTTVTVSSGGGGSGGGGSGGGGSDIVMTQSPDSLTVSLGERT |
| | | | TINCKSSQSVLDSSKNKNSLAWYQQKPGQPPKLLLSWASTRESGIPDRFSGS |
| | | | GSGTDFTLTIDSLQPEDSATYYCQQSAHFPITFGQGTRLEIKSGGGGSEVQL |
| | | | VESGGGLVQPGGSLKLSCAASGFTFNSYAMNWVRQAPGKGLEWVARIRSKYN |
| | | | NYATYYADSVKGRFTISRDDSKNTAYLQMNNLKTEDTAVYYCVRHGNFGNSY |
| | | | VSWWAYWGQGTLVTVSSGGGGSGGGGSGGGGSQTVVTQEPSLTVSPGGTVTL |
| | | | TCGSSTGAVTSGNYPNWVQQKPGQAPRGLIGGTKFLAPGTPARFSGSLLGGK |
| | | | AALTLSGVQPEDEAEYYCVLWYSNRWVFGGGTKLTVL |
| 56 | CD33 AH11 HL × I2C | artificial | QVQLVQSGAEVKKPGESVKVSCKASGYTFTNYGMNWVKQAPGQGLKWMGWIN |
| | HL | | TYTGEPTYADDFKGRVTMTSDTSTSTAYMEISSLRSDDTAVYYCARWSWSDG |
| | | | YYVYFDYWGQGTTVTVSSGGGGSGGGGSGGGGSDIVMTQSPDSLTVSLGERT |
| | | | TINCKSSQSVLDSSKNKNSLAWYQQKPGQPPKLLLSWASTRESGIPDRFSGS |
| | | | GSGTDFTLTIDSLQPEDSATYYCQQSAHFPITFGQGTRLEIKSGGGGSEVQL |
| | | | VESGGGLVQPGGSLKLSCAASGFTFNKYAMNWVRQAPGKGLEWVARIRSKYN |
| | | | NYATYYADSVKDRFTISRDDSKNTAYLQMNNLKTEDTAVYYCVRHGNFGNSY |
| | | | ISYWAYWGQGTLVTVSSGGGGSGGGGSGGGGSQTVVTQEPSLTVSPGGTVTL |
| | | | TCGSSTGAVTSGNYPNWVQQKPGQAPRGLIGGTKFLAPGTPARFSGSLLGGK |
| | | | AALTLSGVQPEDEAEYYCVLWYSNRWVFGGGTKLTVL |
| 57 | CD33 VH of B3 | artificial | QVQLVQSGAEVKKPGESVKVSCKASGYTFTNYGMNWVKQAPGQGLEWMGWIN |
| | | | TYTGETNYADKFQGRVTFTSDTSTSTAYMELRNLKSDDTAVYYCARWSWSDG |
| | | | YYVYFDYWGQGTTVTVSS |
| 58 | CD33 HCDR1 of B3 | artificial | NYGMN |
| 59 | CD33 HCDR2 of B3 | artificial | WINTYTGETNYADKFQG |
| 60 | CD33 HCDR3 of B3 | artificial | WSWSDGYYVYFDY |
| 61 | CD33 VL of B3 | artificial | DIVMTQSPDSMTVSLGERTTINCKSSQSVLDSSTNKNSLAWYQQKPGQPPKL |
| | | | LLSWASTRESGIPDRFSGSGSGTDFTLTIDSLQPEDSATYYCQQSAHFPITF |
| | | | GQGTRLDIK |
| 62 | CD33 LCDR1 of B3 | artificial | KSSQSVLDSSTNKNSLA |
| 63 | CD33 LCDR2 of B3 | artificial | WASTRE |
| 64 | CD33 LCDR3 of B3 | artificial | QQSAHFPIT |
| 65 | CD33 HL of B3 | artificial | QVQLVQSGAEVKKPGESVKVSCKASGYTFTNYGMNWVKQAPGQGLEWMGWIN |
| | | | TYTGETNYADKFQGRVTFTSDTSTSTAYMELRNLKSDDTAVYYCARWSWSDG |
| | | | YYVYFDYWGQGTTVTVSSGGGGSGGGGSGGGGSDIVMTQSPDSMTVSLGERT |
| | | | TINCKSSQSVLDSSTNKNSLAWYQQKPGQPPKLLLSWASTRESGIPDRFSGS |
| | | | GSGTDFTLTIDSLQPEDSATYYCQQSAHFPITFGQGTRLDIK |
| 66 | CD33 B3 HL × H2C HL | artificial | QVQLVQSGAEVKKPGESVKVSCKASGYTFTNYGMNWVKQAPGQGLEWMGWIN |
| | | | TYTGETNYADKFQGRVTFTSDTSTSTAYMELRNLKSDDTAVYYCARWSWSDG |
| | | | YYVYFDYWGQGTTVTVSSGGGGSGGGGSGGGGSDIVMTQSPDSMTVSLGERT |
| | | | TINCKSSQSVLDSSTNKNSLAWYQQKPGQPPKLLLSWASTRESGIPDRFSGS |
| | | | GSGTDFTLTIDSLQPEDSATYYCQQSAHFPITFGQGTRLDIKSGGGGSEVQL |
| | | | VESGGGLVQPGGSLKLSCAASGFTENKYAMNWVRQAPGKGLEWVARIRSKYN |
| | | | NYATYYADSVKDRFTISRDDSKNTAYLQMNNLKTEDTAVYYCVRHGNFGNSY |
| | | | ISYWAYWGQGTLVTVSSGGGGSGGGGSGGGGSQTVVTQEPSLTVSPGGTVTL |
| | | | TCGSSTGAVTSGYYPNWVQQKPGQAPRGLIGGTKFLAPGTPARFSGSLLGGK |
| | | | AALTLSGVQPEDEAEYYCALWYSNRWVFGGGTKLTVL |
| 67 | CD33 B3 HL × F12Q | artificial | QVQLVQSGAEVKKPGESVKVSCKASGYTFTNYGMNWVKQAPGQGLEWMGWIN |
| | HL | | TYTGETNYADKFQGRVTFTSDTSTSTAYMELRNLKSDDTAVYYCARWSWSDG |
| | | | YYVYFDYWGQGTTVTVSSGGGGSGGGGSGGGGSDIVMTQSPDSMTVSLGERT |
| | | | TINCKSSQSVLDSSTNKNSLAWYQQKPGQPPKLLLSWASTRESGIPDRFSGS |
| | | | GSGTDFTLTIDSLQPEDSATYYCQQSAHFPITFGQGTRLDIKSGGGGSEVQL |
| | | | VESGGGLVQPGGSLKLSCAASGFTFNSYAMNWVRQAPGKGLEWVARIRSKYN |
| | | | NYATYYADSVKGRFTISRDDSKNTAYLQMNNLKTEDTAVYYCVRHGNFGNSY |
| | | | VSWWAYWGQGTLVTVSSGGGGSGGGGSGGGGSQTVVTQEPSLTVSPGGTVTL |
| | | | TCGSSTGAVTSGNYPNWVQQKPGQAPRGLIGGTKFLAPGTPARFSGSLLGGK |
| | | | AALTLSGVQPEDEAEYYCVLWYSNRWVFGGGTKLTVL |
| 68 | CD33 B3 HL × I2C HL | artificial | QVQLVQSGAEVKKPGESVKVSCKASGYTFTNYGMNWVKQAPGQGLEWMGWIN |
| | | | TYTGETNYADKFQGRVTFTSDTSTSTAYMELRNLKSDDTAVYYCARWSWSDG |
| | | | YYVYFDYWGQGTTVTVSSGGGGSGGGGSGGGGSDIVMTQSPDSMTVSLGERT |
| | | | TINCKSSQSVLDSSTNKNSLAWYQQKPGQPPKLLLSWASTRESGIPDRFSGS |
| | | | GSGTDFTLTIDSLQPEDSATYYCQQSAHFPITFGQGTRLDIKSGGGGSEVQL |
| | | | VESGGGLVQPGGSLKLSCAASGFTFNKYAMNWVRQAPGKGLEWVARIRSKYN |
| | | | NYATYYADSVKDRFTISRDDSKNTAYLQMNNLKTEDTAVYYCVRHGNFGNSY |
| | | | ISYWAYWGQGTLVTVSSGGGGSGGGGSGGGGSQTVVTQEPSLTVSPGGTVTL |
| | | | TCGSSTGAVTSGNYPNWVQQKPGQAPRGLIGGTKFLAPGTPARFSGSLLGGK |
| | | | AALTLSGVQPEDEAEYYCVLWYSNRWVFGGGTKLTVL |
| 69 | CD33 VH of F2 | artificial | QVQLVQSGAEVKKPGESVKVSCKASGYTFTNYGMNWVKQAPGQGLEWMGWIN |
| | | | TYTGETNYADKFQGRVTFTSDTSTSTAYMELRNLKSDDTAVYYCARWSWSDG |
| | | | YYVYFDYWGQGTTVTVSS |
| 70 | CD33 HCDR1 of F2 | artificial | NYGMN |
| 71 | CD33 HCDR2 of F2 | artificial | WINTYTGETNYADKFQG |
| 72 | CD33 HCDR3 of F2 | artificial | WSWSDGYYVYFDY |
| 73 | CD33 VL of F2 | artificial | DIVMTQSPDSLSVSLGERTTINCKSSQSVLDSSTNKNSLAWYQQKPGQPPKL |
| | | | LLSWASTRESGIPDRFSGSGSGTDFTLTIDSLQPEDSATYYCQQSAHFPITF |
| | | | GQGTRLEIK |
| 74 | CD33 LCDR1 of F2 | artificial | KSSQSVLDSSTNKNSLA |
| 75 | CD33 LCDR2 of F2 | artificial | WASTRES |
| 76 | CD33 LCDR3 of F2 | artificial | QQSAHFPIT |
| 77 | CD33 HL of F2 | artificial | QVQLVQSGAEVKKPGESVKVSCKASGYTFTNYGMNWVKQAPGQGLEWMGWIN |
| | | | TYTGETNYADKFQGRVTFTSDTSTSTAYMELRNLKSDDTAVYYCARWSWSDG |
| | | | YYVYFDYWGQGTTVTVSSGGGGSGGGGSGGGGSDIVMTQSPDSLSVSLGERT |
| | | | TINCKSSQSVLDSSTNKNSLAWYQQKPGQPPKLLLSWASTRESGIPDRFSGS |
| | | | GSGTDFTLTIDSLQPEDSATYYCQQSAHFPITFGQGTRLEIK |
| 78 | CD33 F2 HL × H2C HL | artificial | QVQLVQSGAEVKKPGESVKVSCKASGYTFTNYGMNWVKQAPGQGLEWMGWIN |
| | | | TYTGETNYADKFQGRVTFTSDTSTSTAYMELRNLKSDDTAVYYCARWSWSDG |
| | | | YYVYFDYWGQGTTVTVSSGGGGSGGGGSGGGGSDIVMTQSPDSLSVSLGERT |
| | | | TINCKSSQSVLDSSTNKNSLAWYQQKPGQPPKLLLSWASTRESGIPDRFSGS |
| | | | GSGTDFTLTIDSLQPEDSATYYCQQSAHFPITFGQGTRLEIKSGGGGSEVQL |
| | | | VESGGGLVQPGGSLKLSCAASGFTFNKYAMNWVRQAPGKGLEWVARIRSKYN |
| | | | NYATYYADSVKDRFTISRDDSKNTAYLQMNNLKTEDTAVYYCVRHGNFGNSY |
| | | | ISYWAYWGQGTLVTVSSGGGGSGGGGSGGGGSQTVVTQEPSLTVSPGGTVTL |
| | | | TCGSSTGAVTSGYYPNWVQQKPGQAPRGLIGGTKFLAPGTPARFSGSLLGGK |
| | | | AALTLSGVQPEDEAEYYCALWYSNRWVFGGGTKLTVL |
| 79 | CD33 F2 HL × F12Q | artificial | QVQLVQSGAEVKKPGESVKVSCKASGYTFTNYGMNWVKQAPGQGLEWMGWIN |
| | HL | | TYTGETNYADKFQGRVTFTSDTSTSTAYMELRNLKSDDTAVYYCARWSWSDG |
| | | | YYVYFDYWGQGTTVTVSSGGGGSGGGGSGGGGSDIVMTQSPDSLSVSLGERT |
| | | | TINCKSSQSVLDSSTNKNSLAWYQQKPGQPPKLLLSWASTRESGIPDRFSGS |
| | | | GSGTDFTLTIDSLQPEDSATYYCQQSAHFPITFGQGTRLEIKSGGGGSEVQL |
| | | | VESGGGLVQPGGSLKLSCAASGFTFNSYAMNWVRQAPGKGLEWVARIRSKYN |
| | | | NYATYYADSVKGRFTISRDDSKNTAYLQMNNLKTEDTAVYYCVRHGNFGNSY |
| | | | VSWWAYWGQGTLVTVSSGGGGSGGGGSGGGGSQTVVTQEPSLTVSPGGTVTL |
| | | | TCGSSTGAVTSGNYPNWVQQKPGQAPRGLIGGTKFLAPGTPARFSGSLLGGK |
| | | | AALTLSGVQPEDEAEYYCVLWYSNRWVFGGGTKLTVL |
| 80 | CD33 F2 HL × I2C HL | artificial | QVQLVQSGAEVKKPGESVKVSCKASGYTFTNYGMNWVKQAPGQGLEWMGWIN |
| | | | TYTGETNYADKFQGRVTFTSDTSTSTAYMELRNLKSDDTAVYYCARWSWSDG |
| | | | YYVYFDYWGQGTTVTVSSGGGGSGGGGSGGGGSDIVMTQSPDSLSVSLGERT |
| | | | TINCKSSQSVLDSSTNKNSLAWYQQKPGQPPKLLLSWASTRESGIPDRFSGS |
| | | | GSGTDFTLTIDSLQPEDSATYYCQQSAHFPITFGQGTRLEIKSGGGGSEVQL |
| | | | VESGGGLVQPGGSLKLSCAASGFTFNKYAMNWVRQAPGKGLEWVARIRSKYN |
| | | | NYATYYADSVKDRFTISRDDSKNTAYLQMNNLKTEDTAVYYCVRHGNFGNSY |
| | | | ISYWAYWGQGTLVTVSSGGGGSGGGGSGGGGSQTVVTQEPSLTVSPGGTVTL |
| | | | TCGSSTGAVTSGNYPNWVQQKPGQAPRGLIGGTKFLAPGTPARFSGSLLGGK |
| | | | AALTLSGVQPEDEAEYYCVLWYSNRWVFGGGTKLTVL |
| 81 | CD33 VH of B10 | artificial | QVQLVQSGAEVKKPGESVKVSCKASGYTFTNYGMNWVKQAPGQGLEWMGWIN |
| | | | TYTGEPTYADKFQGRVTMTTDTSTSTAYMEIRNLRSDDTAVYYCARWSWSDG |
| | | | YYVYFDYWGQGTTVTVSS |
| 82 | CD33 HCDR1 of B10 | Artificial | NYGMN |
| 83 | CD33 HCDR2 of B10 | artificial | WINTYTGEPTYADKFQG |
| 84 | CD33 HCDR3 of B10 | artificial | WSWSDGYYVYFDY |
| 85 | CD33 VL of B10 | artificial | DIVMTQSPDSLTVSLGERTTINCKSSQSVLDSSNNKNSLAWYQQKPGQPPKL |
| | | | LLSWASTRESGIPDRFSGSGSGTDFTLTIDGLQPEDSATYYCQQSAHFPITF |
| | | | GQGTRLEIK |
| 86 | CD33 LCDR1 of B10 | artificial | KSSQSVLDSSNNKNSLA |
| 87 | CD33 LCDR2 of B10 | artificial | WASTRES |
| 88 | CD33 LCDR3 of B10 | artificial | QQSAHFPIT |
| 89 | CD33 HL of B10 | artificial | QVQLVQSGAEVKKPGESVKVSCKASGYTFTNYGMNWVKQAPGQGLEWMGWIN |
| | | | TYTGEPTYADKFQGRVTMTTDTSTSTAYMEIRNLRSDDTAVYYCARWSWSDG |
| | | | YYVYFDYWGQGTTVTVSSGGGGSGGGGSGGGGSDIVMTQSPDSLTVSLGERT |
| | | | TINCKSSQSVLDSSNNKNSLAWYQQKPGQPPKLLLSWASTRESGIPDRFSGS |
| | | | GSGTDFTLTIDGLQPEDSATYYCQQSAHFPITFGQGTRLEIK |
| 90 | CD33 B10 HL × H2C | artificial | QVQLVQSGAEVKKPGESVKVSCKASGYTFTNYGMNWVKQAPGQGLEWMGWIN |
| | HL | | TYTGEPTYADKFQGRVTMTTDTSTSTAYMEIRNLRSDDTAVYYCARWSWSDG |
| | | | YYVYFDYWGQGTTVTVSSGGGGSGGGGSGGGGSDIVMTQSPDSLTVSLGERT |
| | | | TINCKSSQSVLDSSNNKNSLAWYQQKPGQPPKLLLSWASTRESGIPDRFSGS |
| | | | GSGTDFTLTIDGLQPEDSATYYCQQSAHFPITFGQGTRLEIKSGGGGSEVQL |
| | | | VESGGGLVQPGGSLKLSCAASGFTFNKYAMNWVRQAPGKGLEWVARIRSKYN |
| | | | NYATYYADSVKDRFTISRDDSKNTAYLQMNNLKTEDTAVYYCVRHGNFGNSY |
| | | | ISYWAYWGQGTLVTVSSGGGGSGGGGSGGGGSQTVVTQEPSLTVSPGGTVTL |
| | | | TCGSSTGAVTSGYYPNWVQQKPGQAPRGLIGGTKFLAPGTPARFSGSLLGGK |
| | | | AALTLSGVQPEDEAEYYCALWYSNRWVFGGGTKLTVL |
| 91 | CD33 B10 HL × F12Q | artificial | QVQLVQSGAEVKKPGESVKVSCKASGYTFTNYGMNWVKQAPGQGLEWMGWIN |
| | HL | | TYTGEPTYADKFQGRVTMTTDTSTSTAYMEIRNLRSDDTAVYYCARWSWSDG |
| | | | YYVYFDYWGQGTTVTVSSGGGGSGGGGSGGGGSDIVMTQSPDSLTVSLGERT |
| | | | TINCKSSQSVLDSSNNKNSLAWYQQKPGQPPKLLLSWASTRESGIPDRFSGS |
| | | | GSGTDFTLTIDGLQPEDSATYYCQQSAHFPITFGQGTRLEIKSGGGGSEVQL |
| | | | VESGGGLVQPGGSLKLSCAASGFTFNSYAMNWVRQAPGKGLEWVARIRSKYN |
| | | | NYATYYADSVKGRFTISRDDSKNTAYLQMNNLKTEDTAVYYCVRHGNFGNSY |
| | | | VSWWAYWGQGTLVTVSSGGGGSGGGGSGGGGSQTVVTQEPSLTVSPGGTVTL |
| | | | TCGSSTGAVTSGNYPNWVQQKPGQAPRGLIGGTKFLAPGTPARFSGSLLGGK |
| | | | AALTLSGVQPEDEAEYYCVLWYSNRWVFGGGTKLTVL |
| 92 | CD33 B10 HL × I2C HL | artificial | QVQLVQSGAEVKKPGESVKVSCKASGYTFTNYGMNWVKQAPGQGLEWMGWIN |
| | | | TYTGEPTYADKFQGRVTMTTDTSTSTAYMEIRNLRSDDTAVYYCARWSWSDG |
| | | | YYVYFDYWGQGTTVTVSSGGGGSGGGGSGGGGSDIVMTQSPDSLTVSLGERT |
| | | | TINCKSSQSVLDSSNNKNSLAWYQQKPGQPPKLLLSWASTRESGIPDRFSGS |
| | | | GSGTDFTLTIDGLQPEDSATYYCQQSAHFPITFGQGTRLEIKSGGGGSEVQL |
| | | | VESGGGLVQPGGSLKLSCAASGFTFNKYAMNWVRQAPGKGLEWVARIRSKYN |
| | | | NYATYYADSVKDRFTISRDDSKNTAYLQMNNLKTEDTAVYYCVRHGNFGNSY |
| | | | ISYWAYWGQGTLVTVSSGGGGSGGGGSGGGGSQTVVTQEPSLTVSPGGTVTL |
| | | | TCGSSTGAVTSGNYPNWVQQKPGQAPRGLIGGTKFLAPGTPARFSGSLLGGK |
| | | | AALTLSGVQPEDEAEYYCVLWYSNRWVFGGGTKLTVL |
| 93 | CD33 VH of E11 | artificial | QVQLVQSGAEVKKPGESVKVSCKASGYTFTNYGMNWVKQAPGQGLEWMGWIN |
| | | | TYTGEPTYADKFQGRVTMTTDTSTSTAYMEIRNLGGDDTAVYYCARWSWSDG |
| | | | YYVYFDYWGQGTSVTVSS |
| 94 | CD33 HCDR1 of E11 | artificial | NYGMN |
| 95 | CD33 HCDR2 of E11 | artificial | WINTYTGEPTYADKFQG |
| 96 | CD33 HCDR3 of E11 | artificial | WSWSDGYYVYFDY |
| 97 | CD33 VL of E11 | artificial | DIVMTQSPDSLTVSLGERTTINCKSSQSVLDSSTNKNSLAWYQQKPGQPPKL |
| | | | LLSWASTRESGIPDRFSGSGSGTDFTLTIDSPQPEDSATYYCQQSAHFPITF |
| | | | GQGTRLEIK |
| 98 | CD33 LCDR1 of E11 | artificial | KSSQSVLDSSTNKNSLA |
| 99 | CD33 LCDR2 of E11 | artificial | WASTRES |
| 100 | CD33 LCDR3 of E11 | artificial | QQSAHFPIT |
| 101 | CD33 HL of E11 | artificial | QVQLVQSGAEVKKPGESVKVSCKASGYTFTNYGMNWVKQAPGQGLEWMGWIN |
| | | | TYTGEPTYADKFQGRVTMTTDTSTSTAYMEIRNLGGDDTAVYYCARWSWSDG |
| | | | YYVYFDYWGQGTSVTVSSGGGGSGGGGSGGGGSDIVMTQSPDSLTVSLGERT |
| | | | TINCKSSQSVLDSSTNKNSLAWYQQKPGQPPKLLLSWASTRESGIPDRFSGS |
| | | | GSGTDFTLTIDSPQPEDSATYYCQQSAHFPITFGQGTRLEIK |
| 102 | CD33 E11 HL × H2C | artificial | QVQLVQSGAEVKKPGESVKVSCKASGYTFTNYGMNWVKQAPGQGLEWMGWIN |
| | HL | | TYTGEPTYADKFQGRVTMTTDTSTSTAYMEIRNLGGDDTAVYYCARWSWSDG |
| | | | YYVYFDYWGQGTSVTVSSGGGGSGGGGSGGGGSDIVMTQSPDSLTVSLGERT |
| | | | TINCKSSQSVLDSSTNKNSLAWYQQKPGQPPKLLLSWASTRESGIPDRFSGS |
| | | | GSGTDFTLTIDSPQPEDSATYYCQQSAHFPITFGQGTRLEIKSGGGGSEVQL |
| | | | VESGGGLVQPGGSLKLSGAASGFTFNKYAMNWVRQAPGKGLEWVARIRSKYN |
| | | | NYATYYADSVKDRFTISRDDSKNTAYLQMNNLKTEDTAVYYCVRHGNFGNSY |
| | | | ISYWAYWGQGTLVTVSSGGGGSGGGGSGGGGSQTVVTQEPSLTVSPGGTVTL |
| | | | TCGSSTGAVTSGYYPNWVQQKPGQAPRGLIGGTKFLAPGTPARFSGSLLGGK |
| | | | AALTLSGVQPEDEAEYYCALWYSNRWVFGGGTKLTVL |
| 103 | CD33 E11 HL × F12Q | artificial | QVQLVQSGAEVKKPGESVKVSCKASGYTFTNYGMNWVKQAPGQGLEWMGWIN |
| | HL | | TYTGEPTYADKFQGRVTMTTDTSTSTAYMEIRNLGGDDTAVYYCARWSWSDG |
| | | | YYVYFDYWGQGTSVTVSSGGGGSGGGGSGGGGSDIVMTQSPDSLTVSLGERT |
| | | | TINCKSSQSVLDSSTNKNSLAWYQQKPGQPPKLLLSWASTRESGIPDRFSGS |
| | | | GSGTDFTLTIDSPQPEDSATYYCQQSAHFPITFGQGTRLEIKSGGGGSEVQL |
| | | | VESGGGLVQPGGSLKLSCAASGFTFNSYAMNWVRQAPGKGLEWVARIRSKYN |
| | | | NYATYYADSVKGRFTISRDDSKNTAYLQMNNLKTEDTAVYYCVRHGNFGNSY |
| | | | VSWWAYWGQGTLVTVSSGGGGSGGGGSGGGGSQTVVTQEPSLTVSPGGTVTL |
| | | | TCGSSTGAVTSGNYPNWVQQKPGQAPRGLIGGTKFLAPGTPARFSGSLLGGK |
| | | | AALTLSGVQPEDEAEYYCVLWYSNRWVFGGGTKLTVL |
| 104 | CD33 E11 HL × I2C HL | artificial | QVQLVQSGAEVKKPGESVKVSCKASGYTFTNYGMNWVKQAPGQGLEWMGWIN |
| | | | TYTGEPTYADKFQGRVTMTTDTSTSTAYMEIRNLGGDDTAVYYCARWSWSDG |
| | | | YYVYFDYWGQGTSVTVSSGGGGSGGGGSGGGGSDIVMTQSPDSLTVSLGERT |
| | | | TINCKSSQSVLDSSTNKNSLAWYQQKPGQPPKLLLSWASTRESGIPDRFSGS |
| | | | GSGTDFTLTIDSPQPEDSATYYCQQSAHFPITFGQGTRLEIKSGGGGSEVQL |
| | | | VESGGGLVQPGGSLKLSCAASGFTFNKYAMNWVRQAPGKGLEWVARIRSKYN |
| | | | NYATYYADSVKDRFTISRDDSKNTAYLQMNNLKTEDTAVYYCVRHGNFGNSY |
| | | | ISYWAYWGQGTLVTVSSGGGGSGGGGSGGGGSQTVVTQEPSLTVSPGGTVTL |
| | | | TCGSSTGAVTSGNYPNWVQQKPGQAPRGLIGGTKFLAPGTPARFSGSLLGGK |
| | | | AALTLSGVQPEDEAEYYCVLWYSNRWVFGGGTKLTVL |
| 105 | CD33 UD H2C HL × | artificial | EVQLVESGGGLVQPGGSLKLSGAASGFTFNKYAMNWVRQAPGKGLEWVARIR |
| | AF5 HL | | SKYNNYATYYADSVKDRFTISRDDSKNTAYLQMNNLKTEDTAVYYCVRHGNF |
| | | | GNSYISYWAYWGQGTLVTVSSGGGGSGGGGSGGGGSQTVVTQEPSLTVSPGG |
| | | | TVTLTCGSSTGAVTSGYYPNWVQQKPGQAPRGLIGGTKFLAPGTPARFSGSL |
| | | | LGGKAALTLSGVQPEDEAEYYCALWYSNRWVFGGGTKLTVLSGGGGSQVQLV |
| | | | QSGAEVKKPGASVKVSCKASGYTFTNYGMNWVKQAPGQGLKWMGWINTYTGE |
| | | | PTYADDFKGRVTMTSDTSTSTAYLELHNLRSDDTAVYYCARWSWSDGYYVYF |
| | | | DYWGQGTTVTVSSGGGGSGGGGSGGGGSDIVMTQSPDSLTVSLGERTTINCK |
| | | | SSQSVLDSSKNKNSLAWYQQKPGQPPKLLLSWASTRESGIPDRFSGSGSGTD |
| | | | FTLTIDSLQPEDSATYYCQQSAHFPITFGQGTRLEIK |
| 106 | CD33 UD F12Q HL × | artificial | EVQLVESGGGLVQPGGSLKLSCAASGFTFNSYAMNWVRQAPGKGLEWVARIR |
| | AF5 HL | | SKYNNYATYYADSVKGRFTISRDDSKNTAYLQMNNLKTEDTAVYYCVRHGNF |
| | | | GNSYVSWWAYWGQGTLVTVSSGGGGSGGGGSGGGGSQTVVTQEPSLTVSPGG |
| | | | TVTLTCGSSTGAVTSGNYPNWVQQKPGQAPRGLIGGTKFLAPGTPARESGSL |
| | | | LGGKAALTLSGVQPEDEAEYYCVLWYSNRWVFGGGTKLTVLSGGGGSQVQLV |
| | | | QSGAEVKKPGASVKVSCKASGYTFTNYGMNWVKQAPGQGLKWMGWINTYTGE |
| | | | PTYADDFKGRVTMTSDTSTSTAYLELHNLRSDDTAVYYCARWSWSDGYYVYF |
| | | | DYWGQGTTVTVSSGGGGSGGGGSGGGGSDIVMTQSPDSLTVSLGERTTINCK |
| | | | SSQSVLDSSKNKNSLAWYQQKPGQPPKLLLSWASTRESGIPDRFSGSGSGTD |
| | | | FTLTIDSLQPEDSATYYCQQSAHFPITFGQGTRLEIK |
| 107 | CD33 UD I2C HL × | artificial | EVQLVESGGGLVQPGGSLKLSCAASGFTENKYAMNWVRQAPGKGLEWVARIR |
| | AF5 HL | | SKYNNYATYYADSVKDRFTISRDDSKNTAYLQMNNLKTEDTAVYYCVRHGNE |
| | | | GNSYISYWAYWGQGTLVTVSSGGGGSGGGGSGGGGSQTVVTQEPSLTVSPGG |
| | | | TVTLTCGSSTGAVTSGNYPNWVQQKPGQAPRGLIGGTKFLAPGTPARFSGSL |
| | | | LGGKAALTLSGVQPEDEAEYYCVLWYSNRWVFGGGTKLTVLSGGGGSQVQLV |
| | | | QSGAEVKKPGASVKVSCKASGYTFTNYGMNWVKQAPGQGLKWMGWINTYTGE |
| | | | PTYADDFKGRVTMTSDTSTSTAYLELHNLRSDDTAVYYCARWSWSDGYYVYF |
| | | | DYWGQGTTVTVSSGGGGSGGGGSGGGGSDIVMTQSPDSLTVSLGERTTINCK |
| | | | SSQSVLDSSKNKNSLAWYQQKPGQPPKLLLSWASTRESGIPDRFSGSGSGTD |
| | | | FTLTIDSLQPEDSATYYCQQSAHFPITFGQGTRLEIK |
| 108 | CD33 E11 HL × I2C HL | artificial | QVQLVQSGAEVKKPGESVKVSCKASGYTFTNYGMNWVKQAPGQGLEWMGWIN |
| | H6 | | TYTGEPTYADKFQGRVTMTTDTSTSTAYMEIRNLGGDDTAVYYCARWSWSDG |
| | | | YYVYFDYWGQGTSVTVSSGGGGSGGGGSGGGGSDIVMTQSPDSLTVSLGERT |
| | | | TINCKSSQSVLDSSTNKNSLAWYQQKPGQPPKLLLSWASTRESGIPDRFSGS |
| | | | GSGTDFTLTIDSPQPEDSATYYCQQSAHFPITFGQGTRLEIKSGGGGSEVQL |
| | | | VESGGGLVQPGGSLKLSCAASGETENKYAMNWVRQAPGKGLEWVARIRSKYN |
| | | | NYATYYADSVKDRFTISRDDSKNTAYLQMNNLKTEDTAVYYCVRHGNFGNSY |
| | | | ISYWAYWGQGTLVTVSSGGGGSGGGGSGGGGSQTVVTQEPSLTVSPGGTVTL |
| | | | TCGSSTGAVTSGNYPNWVQQKPGQAPRGLIGGTKFLAPGTPARESGSLLGGK |
| | | | AALTLSGVQPEDEAEYYCVLWYSNRWVFGGGTKLTVLHHHHHH |
| 109 | HALB | human | DAHKSEVAHRFKDLGEENFKALVLIAFAQYLQQCPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLCMFLYEYARRHPDYSVVLLLRLAKTYETTLEKCCAAA |
| | | | DPHECYAKVFDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALEVDETYVPKEFNAETFTFHADICTLSEKE |
| | | | RQIKKQTALVELVKHKPKATKEQLKAVMDDFAAFVEKCCKADDKETCFAEEG |
| | | | KKLVAASQAALGL |
| 110 | HALB7 | artificial | DAHKSEVAHRFKDLGEENFKALVLIAFAQYLQQCPFEDHVKLVNEVTEFAKT |
| | | | CVADESAENCDKSLHTLFCDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVEDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALEVDETYVPKEFNAGTFTFHADICTLSEKE |
| | | | RQIKKQTALVELVKHKPKATKEQLKAAMDDFAAFVEKCCKADDKETCFAEEG |
| | | | KKLVAASQAALGL |
| 111 | HALB098 | artificial | DAHKSEVAHRFKDLGEENFKALVLIAFAQYLQQCPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVEDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALEVDETYVPKEFNAETFTFHADICTLSEKE |
| | | | RQIKKQTALVELVKHKPKATKEQLKAVMDDFAAFVEKCCKADDKETCFAEEG |
| | | | PKLVAASQAALGL |
| 112 | HALB114 | artificial | DAHKSEVAHRFKDLGEENFKALVLIAFAQYLQQCPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVFDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALDVDETYVPKEFNAETFTFHADICTLSEKE |
| | | | RQIKKQTALVELVKHKPKATKEQLKAVMDDFAAFVEKCCKADDKETCFAEEG |
| | | | PHLVAASKAALGL |
| 113 | HALB254 | artificial | DAHKSEVAHRFKDLGEENFKALVLIAFAQYLQQCPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVEDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALGVDETYVPKEFNAETFTFHADICTLSEKE |
| | | | RQIKKQTALVELVKHKPKATKEQLKAVMDKFAAFVEKCCKADDKETCFAEEG |
| | | | PKLVAASQAALGL |
| 114 | HALB253 | artificial | DAHKSEVAHRFKDLGEENFKALVLIAFAQYLQQCPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVFDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALDVDETYVPKEFNAETFTFHADICTLSEKE |
| | | | RQIKKQTALVELVKHKPKATKEQLKAVMDKFAAFVEKCCKADDKETCFAEEG |
| | | | PKLVAASQAALGL |
| 115 | HALB131 | artificial | DAHKSEVAHRFKDLGEENFKALVLIAFAQYLQQCPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVFDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALEVDETYVPKEFNAETFTFHADICTLSEKE |
| | | | RQIKKQTALVELVKHKPKATKEQLKAVMDKFAAFVEKCCKADDKETCFAEEG |
| | | | PHLVAASQAALGL |
| 116 | HALB135 | artificial | DAHKSEVAHRFKDLGEENFKALVLIAFAQYLQQCPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVEDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALEVDETYVPKEFNAETFTFHADICTLSEKE |
| | | | RQIKKQTALVELVKHKPKATKEQLKAVMDDFAAFVEKCCKADDKETCFAEEG |
| | | | PHLVAASKAALGL |
| 117 | HALB133 | artificial | DAHKSEVAHRFKDLGEENFKALVLIAFAQYLQQCPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVFDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALEVDETYVPKEFNAETFTFHADICTLSEKE |
| | | | RQIKKQTALVELVKHKPKATKEQLKAVMDKFAAFVEKCCKADDKETCFAEEG |
| | | | PKLVAASKAALGL |
| 118 | HALB234 | artificial | DAHKSEVAHRFKDLGEENFKALVLIAFAQYLQQCPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVFDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALDVDETYVPKEFNAETFTFHADICTLSEKE |
| | | | RQIKKQTALVELVKHKPKATKEQLKAVMDDFAAFVEKCCKADDKETCFAEEG |
| | | | PKLVAASKAALGL |
| 119 | HALB C34S | artificial | DAHKSEVAHRFKDLGEENFKALVLIAFAQYLQQSPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVFDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALEVDETYVPKEFNAETFTFHADICTLSEKE |
| | | | RQIKKQTALVELVKHKPKATKEQLKAVMDDFAAFVEKCCKADDKETCFAEEG |
| | | | KKLVAASQAALGL |
| 120 | HALB7 C34S | artificial | DAHKSEVAHRFKDLGEENFKALVLIAFAQYLQQSPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVFDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALEVDETYVPKEFNAGTFTFHADICTLSEKE |
| | | | RQIKKQTALVELVKHKPKATKEQLKAAMDDFAAFVEKCCKADDKETCFAEEG |
| | | | KKLVAASQAALGL |
| 121 | HALB098 C34S | artificial | DAHKSEVAHRFKDLGEENFKALVLIAFAQYLQQSPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVFDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALEVDETYVPKEFNAETFTFHADICTLSEKE |
| | | | RQIKKQTALVELVKHKPKATKEQLKAVMDDFAAFVEKCCKADDKETCFAEEG |
| | | | PKLVAASQAALGL |
| 122 | HALB114 C34S | artificial | DAHKSEVAHRFKDLGEENFKALVLIAFAQYLQQSPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVFDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALDVDETYVPKEFNAETFTFHADICTLSEKE |
| | | | RQIKKQTALVELVKHKPKATKEQLKAVMDDFAAFVEKCCKADDKETCFAEEG |
| | | | PHLVAASKAALGL |
| 123 | HALB254 C34S | artificial | DAHKSEVAHRFKDLGEENFKALVLIAFAQYLQQSPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVFDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALGVDETYVPKEFNAETFTFHADICTLSEKE |
| | | | RQIKKQTALVELVKHKPKATKEQLKAVMDKFAAFVEKCCKADDKETCFAEEG |
| | | | PKLVAASQAALGL |
| 124 | HALB253 C34S | artificial | DAHKSEVAHRFKDLGEENFKALVLIAFAQYLQQSPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVFDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALDVDETYVPKEFNAETFTFHADICTLSEKE |
| | | | RQIKKQTALVELVKHKPKATKEQLKAVMDKFAAFVEKCCKADDKETCFAEEG |
| | | | PKLVAASQAALGL |
| 125 | HALB131 C34S | artificial | DAHKSEVAHRFKDLGEENFKALVLIAFAQYLQQSPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVFDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALEVDETYVPKEFNAETFTFHADICTLSEKE |
| | | | RQIKKQTALVELVKHKPKATKEQLKAVMDKFAAFVEKCCKADDKETCFAEEG |
| | | | PHLVAASQAALGL |
| 126 | HALB135 C34S | artificial | DAHKSEVAHRFKDLGEENFKALVLIAFAQYLQQSPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVFDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALEVDETYVPKEFNAETFTFHADICTLSEKE |
| | | | RQIKKQTALVELVKHKPKATKEQLKAVMDDFAAFVEKCCKADDKETCFAEEG |
| | | | PHLVAASKAALGL |
| 127 | HALB133 C34S | artificial | DAHKSEVAHRFKDLGEENFKALVLIAFAQYLQQSPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVEDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALEVDETYVPKEFNAETFTFHADICTLSEKE |
| | | | RQIKKQTALVELVKHKPKATKEQLKAVMDKFAAFVEKCCKADDKETCFAEEG |
| | | | PKLVAASKAALGL |
| 128 | HALB234 C34S | artificial | DAHKSEVAHRFKDLGEENFKALVLIAFAQYLQQSPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVFDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALDVDETYVPKEFNAETFTFHADICTLSEKE |
| | | | RQIKKQTALVELVKHKPKATKEQLKAVMDDFAAFVEKCCKADDKETCFAEEG |
| | | | PKLVAASKAALGL |
| 129 | HALB C34A | artificial | DAHKSEVAHRFKDLGEENFKALVLIAFAQYLQQAPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVFDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALEVDETYVPKEFNAETFTFHADICTLSEKE |
| | | | RQIKKQTALVELVKHKPKATKEQLKAVMDDFAAFVEKCCKADDKETCFAEEG |
| | | | KKLVAASQAALGL |
| 130 | HALB7 C34A | artificial | DAHKSEVAHRFKDLGEENFKALVLIAFAQYLQQAPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVFDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALEVDETYVPKEFNAGTFTFHADICTLSEKE |
| | | | RQIKKQTALVELVKHKPKATKEQLKAAMDDFAAFVEKCCKADDKETCFAEEG |
| | | | KKLVAASQAALGL |
| 131 | HALB098 C34A | artificial | DAHKSEVAHRFKDLGEENFKALVLIAFAQYLQQAPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVEDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALEVDETYVPKEFNAETFTFHADICTLSEKE |
| | | | RQIKKQTALVELVKHKPKATKEQLKAVMDDFAAFVEKCCKADDKETCFAEEG |
| | | | PKLVAASQAALGL |
| 132 | HALB114 C34A | artificial | DAHKSEVAHRFKDLGEENFKALVLIAFAQYLQQAPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVFDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALDVDETYVPKEFNAETFTFHADICTLSEKE |
| | | | RQIKKQTALVELVKHKPKATKEQLKAVMDDFAAFVEKCCKADDKETCFAEEG |
| | | | PHLVAASKAALGL |
| 133 | HALB254 C34A | artificial | DAHKSEVAHRFKDLGEENFKALVLIAFAQYLQQAPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVFDEFKPLVEEPQNLIKQNCELFEQLGEYKFQNALLVRYTKKVP |
| | | | QVSTPTLVEVSRNLCKVCSKCCKHPEAKRMPCAEDYLSVVLNQLCVLHEKTP |
| | | | VSDRVTKCCTESLVNRRPCFSALGVDETYVPKEFNAETFTFHADICTLSEKE |
| | | | RQIKKQTALVELVKHKPKATKEQLKAVMDKFAAFVEKCCKADDKETCFAEEG |
| | | | PKLVAASQAALGL |
| 134 | HALB253 C34A | artificial | DAHKSEVAHRFKDLGEENFKALVLIAFAQYLQQAPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVFDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALDVDETYVPKEFNAETFTFHADICTLSEKE |
| | | | RQIKKQTALVELVKHKPKATKEQLKAVMDKFAAFVEKCCKADDKETCFAEEG |
| | | | PKLVAASQAALGL |
| 135 | HALB131 C34A | artificial | DAHKSEVAHRFKDLGEENFKALVLIAFAQYLQQAPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVFDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALEVDETYVPKEFNAETFTFHADICTLSEKE |
| | | | RQIKKQTALVELVKHKPKATKEQLKAVMDKFAAFVEKCCKADDKETCFAEEG |
| | | | PHLVAASQAALGL |
| 136 | HALB135 C34A | artificial | DAHKSEVAHRFKDLGEENFKALVLIAFAQYLQQAPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVFDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALEVDETYVPKEFNAETFTFHADICTLSEKE |
| | | | RQIKKQTALVELVKHKPKATKEQLKAVMDDFAAFVEKCCKADDKETCFAEEG |
| | | | PHLVAASKAALGL |
| 137 | HALB133 C34A | artificial | DAHKSEVAHRFKDLGEENFKALVLIAFAQYLQQAPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVFDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALEVDETYVPKEFNAETFTFHADICTLSEKE |
| | | | RQIKKQTALVELVKHKPKATKEQLKAVMDKFAAFVEKCCKADDKETCFAEEG |
| | | | PKLVAASKAALGL |
| 138 | HALB234 C34A | artificial | DAHKSEVAHRFKDLGEENFKALVLIAFAQYLQQAPFEDHVKLVNEVTEFAKT |
| | | | CVADESAENCDKSLHTLFGDKLCTVATLRETYGEMADCCAKQEPERNECFLQ |
| | | | HKDDNPNLPRLVRPEVDVMCTAFHDNEETFLKKYLYEIARRHPYFYAPELLF |
| | | | FAKRYKAAFTECCQAADKAACLLPKLDELRDEGKASSAKQRLKCASLQKFGE |
| | | | RAFKAWAVARLSQRFPKAEFAEVSKLVTDLTKVHTECCHGDLLECADDRADL |
| | | | AKYICENQDSISSKLKECCEKPLLEKSHCIAEVENDEMPADLPSLAADFVES |
| | | | KDVCKNYAEAKDVFLGMFLYEYARRHPDYSVVLLLRLAKTYETTLEKCCAAA |
| | | | DPHECYAKVFDEFKPLVEEPQNLIKQNCELFEQLGEYKFQNALLVRYTKKVP |
| | | | QVSTPTLVEVSRNLGKVGSKCCKHPEAKRMPCAEDYLSVVLNQLCVLHEKTP |
| | | | VSDRVTKCCTESLVNRRPCFSALDVDETYVPKEFNAETFTFHADICTLSEKE |
| | | | RQIKKQTALVELVKHKPKATKEQLKAVMDDFAAFVEKCCKADDKETCFAEEG |
| | | | PKLVAASKAALGL |
| 139 | Peptide linker | artificial | GGGG |
| 140 | Peptide linker | artificial | GGGGS |
| 141 | Peptide linker | artificial | GGGGQ |
| 142 | Peptide linker | artificial | PGGGGS |
| 143 | Peptide linker | artificial | PGGDGS |
| 144 | Peptide linker | artificial | SGGGGS |
| 145 | Peptide linker | artificial | GGGGSGGGS |
| 146 | Peptide linker | artificial | GGGGSGGGGS |
| 147 | Peptide linker | artificial | GGGGSGGGGSGGGGS |
| 148 | CDR-L1 of F6A | artificial | GSSTGAVTSGYYPN |
| 149 | CDR-L2 of F6A | artificial | GTKFLAP |
| 150 | CDR-L3 of F6A | artificial | ALWYSNRWV |
| 151 | CDR-H1 of F6A | artificial | IYAMN |
| 152 | CDR-H2 of F6A | artificial | RIRSKYNNYATYYADSVKS |
| 153 | CDR-H3 of F6A | artificial | HGNFGNSYVSFFAY |
| 154 | CDR-L1 of H2C | artificial | GSSTGAVTSGYYPN |
| 155 | CDR-L2 of H2C | artificial | GTKFLAP |
| 156 | CDR-L3 of H2C | artificial | ALWYSNRWV |
| 157 | CDR-H1 of H2C | artificial | KYAMN |
| 158 | CDR-H2 of H2C | artificial | RIRSKYNNYATYYADSVKD |
| 159 | CDR-H3 of H2C | artificial | HGNFGNSYISYWAY |
| 160 | CDR-L1 of H1E | artificial | GSSTGAVTSGYYPN |
| 161 | CDR-L2 of H1E | artificial | GTKFLAP |
| 162 | CDR-L3 of H1E | artificial | ALWYSNRWV |
| 163 | CDR-H1 of H1E | artificial | SYAMN |
| 164 | CDR-H2 of H1E | artificial | RIRSKYNNYATYYADSVKG |
| 165 | CDR-H3 of H1E | artificial | HGNFGNSYLSFWAY |
| 166 | CDR-L1 of G4H | artificial | GSSTGAVTSGYYPN |
| 167 | CDR-L2 of G4H | artificial | GTKFLAP |
| 168 | CDR-L3 of G4H | artificial | ALWYSNRWV |
| 169 | CDR-H1 of G4H | artificial | RYAMN |
| 170 | CDR-H2 of G4H | artificial | RIRSKYNNYATYYADSVKG |
| 171 | CDR-H3 of G4H | artificial | HGNFGNSYLSYFAY |
| 172 | CDR-L1 of A2J | artificial | RSSTGAVTSGYYPN |
| 173 | CDR-L2 of A2J | artificial | ATDMRPS |
| 174 | CDR-L3 of A2J | artificial | ALWYSNRWV |
| 175 | CDR-H1 of A2J | artificial | VYAMN |
| 176 | CDR-H2 of A2J | artificial | RIRSKYNNYATYYADSVKK |
| 177 | CDR-H3 of A2J | artificial | HGNFGNSYLSWWAY |
| 178 | CDR-L1 of E1L | artificial | GSSTGAVTSGYYPN |
| 179 | CDR-L2 of E1L | artificial | GTKFLAP |
| 180 | CDR-L3 of E1L | artificial | ALWYSNRWV |
| 181 | CDR-H1 of E1L | artificial | KYAMN |
| 182 | CDR-H2 of E1L | artificial | RIRSKYNNYATYYADSVKS |
| 183 | CDR-H3 of E1L | artificial | HGNFGNSYTSYYAY |
| 184 | CDR-L1 of E2M | artificial | RSSTGAVTSGYYPN |
| 185 | CDR-L2 of E2M | artificial | ATDMRPS |
| 186 | CDR-L3 of E2M | artificial | ALWYSNRWV |
| 187 | CDR-H1 of E2M | artificial | GYAMN |
| 188 | CDR-H2 of E2M | artificial | RIRSKYNNYATYYADSVKE |
| 189 | CDR-H3 of E2M | artificial | HRNFGNSYLSWFAY |
| 190 | CDR-L1 of F7O | artificial | GSSTGAVTSGYYPN |
| 191 | CDR-L2 of F7O | artificial | GTKFLAP |
| 192 | CDR-L3 of F7O | artificial | ALWYSNRWV |
| 193 | CDR-H1 of F7O | artificial | VYAMN |
| 194 | CDR-H2 of F7O | artificial | RIRSKYNNYATYYADSVKK |
| 195 | CDR-H3 of F7O | artificial | HGNFGNSYISWWAY |
| 196 | CDR-L1 of F12Q | artificial | GSSTGAVTSGNYPN |
| 197 | CDR-L2 of F12Q | artificial | GTKFLAP |
| 198 | CDR-L3 of F12Q | artificial | VLWYSNRWV |
| 199 | CDR-H1 of F12Q | artificial | SYAMN |
| 200 | CDR-H2 of F12Q | artificial | RIRSKYNNYATYYADSVKG |
| 201 | CDR-H3 of F12Q | artificial | HGNFGNSYVSWWAY |
| 202 | CDR-L1 of I2C | artificial | GSSTGAVTSGNYPN |
| 203 | CDR-L2 of I2C | artificial | GTKFLAP |
| 204 | CDR-L3 of I2C | artificial | VLWYSNRWV |
| 205 | CDR-H1 of I2C | artificial | KYAMN |
| 206 | CDR-H2 of I2C | artificial | RIRSKYNNYATYYADSVKD |
| 207 | CDR-H3 of I2C | artificial | HGNFGNSYISYWAY |

1. A bispecific antibody construct comprising a first binding domain specifically binding to CD33 and a second binding domain specifically binding to CD3 preferably for use in a method for the treatment of myeloid leukemia, wherein the bispecific antibody construct is administered in one or more treatment cycles, wherein at least one treatment cycle comprises more than 14 days of administration of the bispecific antibody construct in at least three different dosages applying at least two dosage steps, optionally followed by a period without administration of the construct,
wherein the bispecific antibody construct is administered in at least one of the one or more treatment cycles according to a schedule comprising the following steps:
(a) administration of a first dosage of the bispecific antibody construct, followed by
(b) administration of a second dosage of the bispecific antibody construct, wherein said second dosage exceeds said first dosage, followed by
(c) administration of a third dosage of the bispecific antibody construct, wherein said third dosage exceeds said second dosage, optionally followed by
(d) administration of a forth dosage of the bispecific antibody construct, wherein said optional forth dosage exceeds said third dosage.

2. The bispecific antibody construct for the use according to claim 1, wherein the time of administering the bispecific antibody construct in one treatment cycle is at least 15 days, preferably 15 to 60 days, more preferably 28 to 56 days, preferably 28 days.

3. The bispecific antibody construct for the use according to claim 1 or 2, wherein the first dosage in step (a) is at least μg per day, preferably in the range of 5 to 20 μg per day, more preferably 10 μg per day, the second dosage in step (b) is at least 30 μg per day, preferably in the range of 30 to 240 μg per day, more preferably 60 or 240 μg per day and the third dosage in step (c) and the optional forth dosage in step (d) is at least 240 μg per day, preferably in the range of 240 to 1500 μg per day, preferably in the range of 240 to 960 μg per day, more preferably in the range of 480 to 960 μg per day.

4. The bispecific antibody construct for the use according to claim 1, wherein the period of administration of the first dosage in step (a) is 1 to 4 days, preferably 2 or 3 days, the period of administration of the second dosage in step (b) is 2 to 5 days, preferably 2 or 3 days, and the period of administration of the third dosage in step (c) and the optional forth dosage in step (d) together is 7 to 52 days, preferably 14 to 52 days, more preferably 22, 23 or 52 days.

5. The bispecific antibody construct for the use according to any one of claims 2 to 4, wherein the treatment of the myeloid leukemia comprises two or more treatment cycles, preferably two, three, four, five, six or seven treatment cycles, whereof at least one, two, three, four five, six or seven treatment cycles comprise more than 14 days of bispecific antibody construct administration.

6. The bispecific antibody construct for the use according to any one of claims 2 to 5, wherein at least one treatment cycle is followed by the period without administration of the construct, preferably at least 1, 2, 3, 4, 5, 6, 7, 8, 9, 10, 11, 12, 13 or 14 days without treatment.

7. The bispecific antibody construct for the use according to any one of claims 2 to 5, wherein at least one treatment cycle is not followed by the period without administration of the construct.

8. The bispecific antibody construct for the use according to any of claims 5 to 7, wherein only the first cycle of the treatment comprises the administration according to step (a), whereas the following cycles start with the dose according to step (b).

9. The bispecific antibody construct for the use according to any one of claims 1 to 8, wherein the construct is a single chain bispecific antibody construct.

10. The bispecific antibody construct for the use according to any one of claims 1 to 8, wherein the first binding domain of the bispecific antibody construct comprises groups of six CDRs selected from the group consisting of SEQ ID NOs: 10 to 12 and 14 to 16, 22 to 24 and 26 to 28, 34 to 36 and 38 to 40, 46 to 48 and 50 to 52, 58 to 60 and 62 to 64, 70 to 72 and 74 to 76, 82 to 84 and 86 to 88, 94 to 96 an 98 to 100, preferably 94 to 96 an 98 to 100.

11. The bispecific antibody construct for the use according to any one of claims 1 to 8, wherein the second binding domain of the bispecific antibody construct comprises groups of six CDRs selected from the group consisting of SEQ ID NOs: 148-153, 154-159, 160-165, 166-171, 172-177, 178-183, 184-189, 190-195, 196-201 and 202-207, preferably 202-207.

12. The bispecific antibody construct for the use according to claims 1 to 11, wherein the bispecific antibody construct is a single chain construct comprising an amino acid sequence selected from the group consisting of SEQ ID NOs: 18, 19, 20, 30, 31, 32, 42, 43, 44, 54, 55, 56, 66, 67, 68, 78, 79, 80, 90, 91, 92, 102, 103, 104, 105, 106, 107 and 108, preferably selected from the group consisting of SEQ ID NOs: 104, 105, 106, 107 and 108, more preferably SEQ ID NO 104.

13. The bispecific antibody construct for the use according to any one of the preceding claims, wherein the bispecific antibody construct is administered in combination with a PD-1 inhibitor, a PDL-1 inhibitor and/or one or more epigenetic factors selected from the group consisting of histone deacetylase (HDAC) inhibitors, DNA methyltransferase (DNMT) I inhibitors, hydroxyurea, Granulocyte-Colony Stimulating Factor (G-CSF), histone demethylase inhibitors and ATRA (All Trans-retinoic acid) and wherein:
(a) the PD-1 inhibitor, a PDL-1 inhibitor and/or one or more epigenetic factors are administered prior to the administration of the bispecific antibody construct;
(b) the PD-1 inhibitor, a PDL-1 inhibitor and/or one or more epigenetic factors are administered subsequent to the administration of the bispecific antibody construct; or
(c) the PD-1 inhibitor, a PDL-1 inhibitor and/or one or more epigenetic factors and the bispecific antibody construct are administered simultaneously.

14. The bispecific antibody construct for the use according to claim 13, wherein the PD-1 inhibitor, PDL-1 inhibitor, and/or one or more epigenetic factors are administered up to seven days prior to the administration of the bispecific antibody construct.

15. The bispecific antibody construct for the use according to claim 14, wherein the epigenetic factor is hydroxyurea.

16. The bispecific antibody construct for the use according to any one of the preceding claims, wherein the myeloid leukemia is selected from the group consisting of acute myeloblastic leukemia, preferably relapsed or refractory acute myeloid leukemia, chronic neutrophilic leukemia, myeloid dendritic cell leukemia, accelerated phase chronic myelogenous leukemia, acute myelomonocytic leukemia, juvenile myelomonocytic leukemia, chronic myelomonocytic leukemia, acute basophilic leukemia, acute eosinophilic leukemia, chronic eosinophilic leukemia, acute megakaryoblastic leukemia, essential thrombocytosis, acute erythroid leukemia, polycythemia vera, myelodysplastic syndrome, acute panmyeloic leukemia, myeloid sarcoma, and mixed phenotypic acute leukemia.

17. A method for the treatment of myeloid leukemia in a patient in need thereof comprising administering a bispecific antibody construct comprising a first binding domain specifically binding to CD33 and a second binding domain specifically binding to CD3 in one or more treatment cycles, wherein the at least one treatment cycle comprises more than 14 days of administration of the bispecific antibody construct in at least three different dosages applying at least two dosage steps,
wherein the bispecific antibody construct is administered in one treatment cycle according to a schedule comprising the following steps:
(a) administration of a first dosage of the bispecific antibody construct, followed by
(b) administration of a second dosage of the bispecific antibody construct, wherein said second dosage exceeds said first dosage, followed by
(c) administration of a third dosage of the bispecific antibody construct, wherein said third dosage exceeds said second dosage, optionally followed by
(d) administration of a forth dosage of the bispecific antibody construct, wherein said optional forth dosage exceeds said third dosage, optionally followed by a period of at without administration of the construct.

18. The method according to claim 14, wherein the time of administering the bispecific antibody construct in one treatment cycle is at least 15 days, preferably 15 to 60 days, more preferably 28 to 56 days, preferably 28 days.

19. The method according to claim 17 or 18, wherein the first dosage in step (a) is at least 5 μg per day, preferably in the range of 5 to 20 μg per day, more preferably 10 μg per day, the second dosage in step (b) is at least 30 μg per day, preferably in the range of 30 to 240 μg per day, preferably 60 or 240 μg per day and the third dosage in step (c) and the optional forth dosage in optional step (d) is at least 240 μg per day, preferably in the range of 120 to 1500 μg per day, preferably 240 to 960 μg per day, more preferably 480 to 960 μg per day.

20. The method according to any one of claims 17 to 19, wherein the period of administration of the first dosage in step (a) is 1 to 4 days, preferably 2 or 3 days, the period of administration of the second dosage in step (b) is 2 to 5 days, preferably 2 or 3 days, and the period of administration of the third and the optional forth dose in step (c) and optional step (d) is 7 to 52 days, preferably 14 to 23 days, more preferably 22, 23, 50 or 52 days.

21. The method according to any one of claims 17 to 20, wherein the treatment of the myeloid leukemia comprises two or more treatment cycles, preferably 2, 3, 4, 5, 6 or 7 treatment cycles, whereof at least 1, 2, 3, 4, 5, 6 or 7 treatment cycles comprise more than 14 days of bispecific antibody construct administration.

22. The method according to any one of claims 17 to 21, wherein the treatment is followed by the period without administration of the bispecific antibody construct, preferably at least 1, 2, 3, 4, 5, 6, 7, 8, 9, 10, 11, 12, 13, or 14 days without treatment.

23. The method according to any one of claims 17 to 21, wherein the treatment is followed by the period of at least 14 days without administration of the bispecific antibody construct.

24. The method according to any one of claims 17 to 23, wherein only the first cycle of the treatment comprises the administration according to step (a), whereas the following cycles start with the dose according to step (b).

25. The method according to any one of the preceding claims 17 to 23, wherein the construct is a single chain bispecific antibody construct.

26. The method according to any one of the preceding claims, wherein the first binding domain of the bispecific antibody construct comprises groups of six CDRs selected from the group consisting of SEQ ID NOs: 10 to 12 and 14 to 16, 22 to 24 and 26 to 28, 34 to 36 and 38 to 40, 46 to 48 and 50 to 52, 58 to 60 and 62 to 64, 70 to 72 and 74 to 76, 82 to 84 and 86 to 88, 94 to 96 an 98 to 100, preferably 94 to 96 an 98 to 100.

27. The method according to any one of claims 17 to 26, wherein the second binding domain of the bispecific antibody construct comprises groups of six CDRs selected from the group consisting of SEQ ID NOs: 148-153, 154-159, 160-165, 166-171, 172-177, 178-183, 184-189, 190-195, 196-201 and 202-207, preferably 202-207.

28. The method according to any one of claims 17 to 27, wherein the bispecific antibody construct is a single chain construct comprising an amino acid sequence selected from the group consisting of SEQ ID NOs: 18, 19, 20, 30, 31, 32, 42, 43, 44, 54, 55, 56, 66, 67, 68, 78, 79, 80, 90, 91, 92, 102, 103, 104, 105, 106, 107 and 108, preferably selected from the group consisting of SEQ ID NOs: 104, 105, 106, 107 and 108, more preferably SEQ ID NO: 104.

29. The method according to any one of claims 17 to 28, further comprising administering at least one PD-1 inhibitor, a PDL-1 inhibitor and/or one or more epigenetic factors selected from the group consisting of histone deacetylase (HDAC) inhibitors, DNA methyltransferase (DNMT) I inhibitors, hydroxyurea, Granulocyte-Colony Stimulating Factor (G-CSF), histone demethylase inhibitors and ATRA (All Trans-retinoic acid) and wherein the at least one PD-1 inhibitor, a PDL-1 inhibitor and/or one or more epigenetic factors are administered:
(a) prior to the administration of the bispecific antibody construct;
(b) subsequent to the administration of the bispecific antibody construct; or
(c) simultaneously with the bispecific antibody construct.

30. The method according to any one of claims 17 to 29, wherein the one PD-1 inhibitor, PDL-1 inhibitor or one or more epigenetic factors are administered up to seven days prior to the administration of the bispecific antibody construct.

31. The method according to any one of claims 17 to 30, wherein the epigenetic factor is hydroxyurea.

32. The method according to any one of claims 17 to 31, wherein the myeloid leukemia is selected from the group consisting of acute myeloblastic leukemia, preferably relapsed or refractory acute myeloid leukemia, chronic neutrophilic leukemia, myeloid dendritic cell leukemia, accelerated phase chronic myelogenous leukemia, acute myelomonocytic leukemia, juvenile myelomonocytic leukemia, chronic myelomonocytic leukemia, acute basophilic leukemia, acute eosinophilic leukemia, chronic eosinophilic leukemia, acute megakaryoblastic leukemia, essential thrombocytosis, acute erythroid leukemia, polycythemia vera, myelodysplastic syndrome, acute panmyeloic leukemia, myeloid sarcoma, and mixed phenotypic acute leukemia acute biphenotypic leukaemia.

33. Use of a bispecific antibody construct comprising a first binding domain specifically binding to CD33 and a second binding domain specifically binding to CD3 preferably in a method for the treatment of myeloid leukemia, wherein the bispecific antibody construct is administered in one or more treatment cycles, wherein at least one treatment cycle comprises more than 14 days of administration of the bispecific antibody construct in at least three different dosages applying at least two dosage steps, optionally followed by a period without administration of the bispecific antibody construct,
wherein the bispecific antibody construct is administered in at least one of the one or more treatment cycle according to a schedule comprising the following steps:
(a) administration of a first dosage of the bispecific antibody construct, followed by
(b) administration of a second dosage of the bispecific antibody construct, wherein said second dosage exceeds said first dosage, followed by
(c) administration of a third dosage of the bispecific antibody construct, wherein said third dosage exceeds said second dosage, optionally followed by
(d) administration of a forth dosage of the bispecific antibody construct, wherein said optional forth dosage exceeds said third dosage.
